(19) United States
(12) Patent Application Publication
(10) Pub. No.: US 2025/0057829 A1
(43) Pub. Date: Feb. 20, 2025

(54) INHIBITORS OF TRPC6 FOR TREATING FOCAL SEGMENTAL GLOMERULOSCLEROSIS

(71) Applicant: Boehringer Ingelheim International GmbH, Ingelheim am Rhein (DE)

(72) Inventors: Roberto Matias NORDABY, Ingelheim (DE); Nima SOLEYMANLOU, Grand Island, NY (US)

(21) Appl. No.: 18/806,773

(22) Filed: Aug. 16, 2024

Related U.S. Application Data
(60) Provisional application No. 63/533,364, filed on Aug. 18, 2023.

(51) Int. Cl. A61K 31/4545; A61K 31/496; A61K 31/499; A61K 31/4995; A61K 31/501; A61K 31/5386

(52) U.S. Cl. CPC A61K 31/4545; A61K 31/496; A61K 31/499; A61K 31/4995; A61K 31/501; A61K 31/5386

(57) ABSTRACT

Disclosed are methods for treating focal segmental glomerulosclerosis (FSGS), comprising administering to a patient in need thereof a pharmaceutically effective amount of a compound of formula (I),

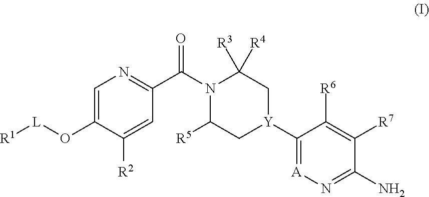

wherein R¹ to R⁷, A, L and Y are as defined herein. Also disclosed are pharmaceutical compositions comprising the compound of formula (I) and their use for treating FSGS.

### FIELD OF THE INVENTION

The present inventions relate to inhibitors of transient receptor potential C6 ion channel (TRPC6) for treating patients having focal segmental glomerulosclerosis (FSGS).

### BACKGROUND OF THE INVENTION

Focal segmental glomerulosclerosis (FSGS) is a leading glomerular cause of End Stage Kidney Disease (ESKD) in the United States. FSGS refers to a histologic pattern that is a characteristic of perhaps distinct underlying etiologies sharing a common theme of podocyte injury and depletion. See Rosenberg A Z and Kopp J B, “Focal segmental glomerulosclerosis,” Clin J Am Soc Nephrol 2017; 12(3):502-517.

FSGS is characterized by histologic lesions as opposed to a specific disease. FSGS is a pathophysiological entity which commonly explains the onset of nephrotic syndrome in adult or pediatric patients. Histological abnormalities contain sclerosis in segmental (parts) of focal (some) glomeruli as assessed by microscopic investigation of kidney biopsies.

FSGS is a frequently found histopathologic lesion in adults with nephrotic syndrome within the United States accounting for 35% of all cases and >50% among African Americans. See Haas M et al., “Changing etiologies of unexplained adult nephrotic syndrome: a comparison of renal biopsy findings from 1976-1979 and 1995-1997,” Am J Kidney Dis 1997; 30(5):621-631 and Kitiyakara C et al., “Twenty-one-year trend in ESRD due to focal segmental glomerulosclerosis in the United States,” Am J Kidney Dis 2004; 44(5):815-825.

The classification of FSGS in specific categories is based on various etiologies as defined below:

Primary (idiopathic) FSGS: Frequently presenting with nephrotic syndrome.

Secondary FSGS (sFSGS) or also referred to as adaptive FSGS: Often presenting with non-nephrotic proteinuria and, commonly, with some extent of kidney function impairment. This category is a common adaptive response to hyperfiltration or glomerular hypertrophy and disorders characterized by renal vasodilation and/or kidney mass reduction (e.g. unilateral renal agenesis). Drug- or toxin-induced (e.g. heroin, interferon, pamidronate) and viral-induced (especially HIV) pathologies account for the other causes of sFSGS.

Genetic (familial) FSGS: Presents generally in early childhood with substantial nephrotic syndrome and proteinuria or with less severe proteinuria in adolescence or adulthood.

FSGS classification will depend on a multitude of assessments including clinical history, laboratory testing, kidney biopsy, and in some cases genetic testing. While considerable progress has been achieved with the clinical understanding of FSGS, research is still needed to identify plasma factor(s) believed to be responsible for primary FSGS, to assess the clinical utility of routine genetic testing, and to find more effective and safer therapeutic interventions for FSGS. See Rosenberg A Z and Kopp J B, “Focal segmental glomerulosclerosis,” Clin J Am Soc Nephrol 2017; 12(3):502-517.

A possibly important mechanism related to glomerular dysfunction in proteinuric diseases could be the calcium overload of the podocyte. In subjects with TRPC6 mutations increased podocyte foot process detachment and loss has been observed as a consequence of disruption of the glomerular filtration barrier. Jiang L et al., “Over-expressing transient receptor potential cation channel 6 in podocytes induces cytoskeleton rearrangement through increases of intracellular Ca2+ and RhoA activation,” Exp Biol Med (Maywood) 2011; 236:184-193 and Tian D et al., “Antagonistic regulation of actin dynamics and cell motility by TRPC5 and TRPC6 channels,” Sci Signal 2010; 3(145):ra77. It is hypothesized that increased TRPC6 activity could be a principal mechanism in proteinuric kidney disease driving progression to ESKD. Therefore, the use of a TRPC6 inhibitor, may be a novel treatment option by limiting TRPC6 channel activity in case of pathological Ca2+ entry which should result in preserved podocyte function and reduced podocyte loss.

FSGS is one of the most common forms of acquired glomerular disease leading to ESKD and it is one of the most important causes of acquired chronic kidney disease in children and adults. Kiffel J et al., “Focal segmental glomerulosclerosis and chronic kidney disease in pediatric patients,” Adv Chronic Kidney Dis 2011; 18(5):332-338. Based on a clear biological link between FSGS and gain of function mutations of TRPC6 in this disease and the TRPC6 inhibitory mechanism of action, treatment with an inhibitor of TRPC6 is expected to reduce proteinuria in FSGS thereby reducing disease burden and potential progression.

TRPC6 is expressed in several renal cell types, including podocytes which are key cells for glomerular filtration function of the kidney. Multiple gain of function mutations in TRPC6 have been demonstrated to cause FSGS by elevating intracellular calcium concentration in podocytes and inducing cytoskeletal rearrangements. This has been linked to podocyte apoptosis, foot process detachment and loss of podocytes, leading to disruption of the glomerular filtration barrier. The modulation of TRPC6 activity should therefore have the potential to improve both podocyte function and survival in proteinuric glomerular diseases and specifically in FSGS.

### SUMMARY OF THE INVENTION

In one embodiment (Embodiment One), the invention relates to methods for reducing the level of proteinuria and/or preserving renal function in a patient having focal segmental glomerulosclerosis (FSGS), comprising administering to the patient in need thereof a pharmaceutically effective amount of a compound of formula (I),

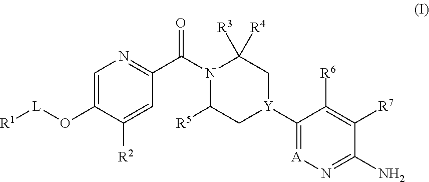

wherein
L is absent or is methylene or ethylene;
Y is CH or N;
A is CH or N;
R¹ is selected from the group consisting of:
C₁₋₆alkyl optionally substituted with 1 to 3 groups independently selected from the group consisting of halo, C₃₋₆cycloalkyl and OC₃₋₆cycloalkyl;
phenyl optionally substituted with 1 to 3 groups independently selected from the group consisting of CF₃, halo, C₃₋₆cycloalkyl, OC₃₋₆cycloalkyl, and OC₁₋₆alkyl; wherein said OC₁₋₆alkyl may be optionally substituted with one to three halo; and
C₃₋₆cycloalkyl optionally substituted with 1 to 3 groups independently selected from the group consisting of halo and C₁₋₆alkyl optionally substituted with 1 to 3 halo;
R² is selected from the group consisting of H, C₁₋₆alkyl, OCF₃, C₃₋₆cycloalkyl, OC₁₋₆alkyl, and OC₃₋₆cycloalkyl;
R³ is selected from the group consisting of H, C₁₋₆alkyl, C₃₋₆cycloalkyl, and OC₃₋₆cycloalkyl; wherein each of the C₁₋₆alkyl, C₃₋₆cycloalkyl, or OC₃₋₆cycloalkyl of the R³ group may independently be optionally substituted with one to three groups each independently selected from the group consisting of halo, OH, OC₁₋₆alkyl, SC₁₋₆alkyl, and N(C₁₋₆alky)₂; and wherein one to three carbon atoms of the C₁₋₆alkyl of the R³ group may optionally be replaced one or two moieties selected from the group consisting of NH, N(C₁₋₆alkyl), O, and S;
R⁴ and R⁵ are each independently selected from the group consisting of H and C₁₋₆alkyl; or
R³ and R⁴ together with the atom to which they are attached may join to form a 3-membered carbocyclyl ring; or
R³ and R⁵ together with the atoms to which they are attached may join to form a 3- to 9-membered bicyclic ring, wherein said 3- to 9-membered bicyclic ring may optionally contain one to three heteroatoms selected from the group consisting of N, O, and S;
R⁶ is selected from the group consisting of H, C₁₋₆alkyl, CN, CF₃, OCF₃, C₃₋₆cycloalkyl, OC₁₋₆alkyl, and OC₃₋₆cycloalkyl;
R⁷ is selected from the group consisting of H and OC₁₋₆alkyl;
or a pharmaceutically acceptable salt thereof.

In another embodiment, (Embodiment Two), the invention relates to the method according to Embodiment One, wherein
R¹ is selected from the group consisting of:
C₁₋₆alkyl optionally substituted with 1 to 3 groups independently selected from the group consisting of halo and C₃₋₆cycloalkyl;
phenyl optionally substituted with 1 to 3 groups independently selected from the group consisting of CF₃, halo, OC₃₋₆cycloalkyl, and OC₁₋₆alkyl; wherein said OC₁₋₆alkyl may be optionally substituted with one to three halo; and
C₃₋₆cycloalkyl optionally substituted with 1 to 3 halo groups;
R² is OC₁₋₆alkyl;
R³ is selected from the group consisting of H and C₁₋₆alkyl optionally substituted with OH or OC₁₋₆alkyl,
R⁴ is H;
R⁵ is H; or
R³ and R⁴ together with the atom to which they are attached may join to form a 3-membered carbocyclyl ring; or
R³ and R⁵ together with the atoms to which they are attached may join to form a 3- to 9-membered bicyclic ring, wherein said 3- to 9-membered bicyclic ring may optionally contain one to three heteroatoms selected from the group consisting of N and O;
R⁶ is selected from the group consisting of H, C₁₋₆alkyl, OC₁₋₆alkyl, and OC₃₋₆cycloalkyl; and
R⁷ is selected from the group consisting of H and OC₁₋₆alkyl;
or a pharmaceutically acceptable salt thereof.

In another embodiment, (Embodiment Three), the invention relates to the method according to Embodiment One, wherein
A is CH and Y is N; or
A is CH and Y is CH; or
A is N and Y is CH;
or a pharmaceutically acceptable salt thereof.

In another embodiment, (Embodiment Four), the invention relates to the method according to Embodiment One, wherein
R¹ is phenyl optionally substituted with a group selected from the group consisting of CF₃, halo, OC₃₋₆cycloalkyl, and OC₁₋₆alkyl; wherein the OC₁₋₆alkyl may be optionally substituted with one to three halo;
R² is OC₁₋₆alkyl;
R³ is selected from the group consisting of H and C₁₋₆alkyl optionally substituted with OH or OC₁₋₆alkyl;
R⁴ is H;
R⁵ is H; or
R³ and R⁴ can together with the atom to which they are attached may join to form a 3-membered carbocyclyl ring, or
R³ and R⁵ together with the atoms to which they are attached may join to form a 3- to 9-membered bicyclic ring, wherein said 3- to 9-membered bicyclic ring may optionally contain one to three heteroatoms selected from the group consisting of N and O;
R⁶ is selected from the group consisting of H, C₁₋₆alkyl, OC₁₋₆alkyl, and OC₃₋₆cycloalkyl;
R⁷ is selected from the group consisting of H and OC₁₋₆alkyl;
or a pharmaceutically acceptable salt thereof.

In another embodiment, (Embodiment Five), the invention relates to the method according to Embodiment One, wherein
R¹ is phenyl optionally substituted with a group selected from the group consisting of CF₃, OCF₃, F, and methoxy;
R² is selected from the group consisting of methoxy or ethoxy;
R³ is selected from the group consisting of H, C₁₋₆alkyl, 2-hydroxymethyl, methoxymethyl, and 1-hydroxyethyl;
R⁴ is H;
R⁵ is H; or
R³ and R⁵ together with the atoms to which they are attached may join to form a 3- to 9-membered bicyclic ring, wherein said 3- to 9-membered bicyclic ring may optionally contain one to three heteroatoms selected from the group consisting of N, O, and S;
R⁶ is selected from the group consisting of H, methyl, methoxy, ethoxy, propoxy, and cyclopropyloxy; and
R⁷ is selected from the group consisting of H and methoxy;
or a pharmaceutically acceptable salt thereof.

In another embodiment, (Embodiment Six), the invention relates to the method according to Embodiment One, wherein
R¹ together with L represent a group selected from the group consisting of phenyl, 4-chlorophenyl, 4-fluorophenyl, 4-methoxyphenyl, 4-isopropoxyphenyl, 4-trifluoromethylphenyl, 4-difluoromethoxyphenyl 4-cyclopropyloxyphenyl, cyclopropyl, cyclopentyl, cyclohexyl, benzyl, 2-fluorobenzyl, and phenylethyl; and
R² is methoxy or ethoxy;
or a pharmaceutically acceptable salt thereof.

In another embodiment, (Embodiment Seven), the invention relates to the method according to Embodiment One, wherein
Y is CH and A is N;
R¹ together with L represent a group selected from the group consisting of phenyl, 4-chlorophenyl, 4-fluorophenyl, 4-methoxyphenyl, 4-isopropoxyphenyl, 4-trifluoromethylphenyl, 4-difluoromethoxyphenyl, 4-cyclopropoxyphenyl, benzyl, 2-fluorobenzyl, and phenylethyl;
R² is methoxy or ethoxy;
R³, R⁴ and R⁵ are each H;
R⁶ is H, methyl, methoxy, or ethoxy; and
R⁷ is H;
or a pharmaceutically acceptable salt thereof.

In another embodiment, (Embodiment Eight), the invention relates to the method according to Embodiment One, wherein
Y is CH and A is CH;
R¹ together with L represent a group selected from the group consisting of phenyl, 4-chlorophenyl, 4-fluorophenyl, 4-methoxyphenyl, 4-trifluoromethylphenyl, cyclopentyl, cyclohexyl, benzyl, 2-fluorobenzyl, and phenylethyl;
R² is methoxy or ethoxy;
R³, R⁴ and R⁵ are each H;
R⁶ is H, methyl, methoxy or ethoxy; and
R⁷ is H;
or a pharmaceutically acceptable salt thereof.

In another embodiment, (Embodiment Nine), the invention relates to the method according to Embodiment One, wherein
Y is N and A is CH;
R¹ together with L represent a group selected from the group consisting of phenyl, and 4-fluorophenyl;
R² is methoxy;
R³ is selected from the group consisting of H, 2-hydroxymethyl, and hydroxyethyl,
R⁴ is H;
R⁵ is H; or
R³ and R⁵ together with the atoms to which they are attached may join to form a 3- to 9-membered bicyclic ring, wherein said 3- to 9-membered bicyclic ring may optionally contain one to three heteroatoms selected from the group consisting of N, O, and S;
R⁶ is selected from the group consisting of H and methoxy; and
R⁷ is H;
or a pharmaceutically acceptable salt thereof.

In another embodiment, (Embodiment Ten), the invention relates to the method according to Embodiment One, wherein
R¹ is C₁₋₆alkyl optionally substituted with 1 to 3 groups independently selected from the group consisting of halo and C₃₋₆cycloalkyl;
R² is OC₁₋₆alkyl;
R³, R⁴ and R⁵ are each H;
R⁶ is selected from the group consisting of H, C₁₋₆alkyl, and OC₁₋₆alkyl; and
R⁷ is H;
or a pharmaceutically acceptable salt thereof.

In another embodiment, (Embodiment Eleven), the invention relates to the method according to Embodiment One, wherein
R¹ together with L represent a group selected from the group consisting of ethyl, propyl, isopropyl, isobutyl, cyclopropylmethyl, cyclobutylmethyl, 2,2-dimethylpropyl, 1-methylcyclopropylmethyl, 1-fluoromethylcyclopropylmethyl, 1-cyclopropylethyl, 2-cyclopropylethyl, cyclopentyl, cyclohexyl, 2,2-difluorocyclobutylmethyl, 3,3-difluorocyclobutylmethyl, 3-(trifluoromethyl)cyclobutylmethyl, and 3,3,3-trifluoro-2-methyl-propyl;
R² is methoxy;
R³, R⁴ and R⁵ are each H;
R⁶ is selected from the group consisting of H, methyl, and methoxy; and
R⁷ is H;
or a pharmaceutically acceptable salt thereof.

In another embodiment, (Embodiment Twelve), the invention relates to the method according to Embodiment One, wherein
Y is CH and A is N;
R¹ together with L represent a group selected from the group consisting of propyl, isopropyl, isobutyl, cyclopropylmethyl, cyclobutylmethyl, 2,2-dimethylpropyl, 1-cyclopropylethyl, and 2-cyclopropylethyl;
R² is methoxy;
R³, R⁴ and R⁵ are each H;
R⁶ is selected from the group consisting of H, methyl, and methoxy; and
R⁷ is H;
or a pharmaceutically acceptable salt thereof.

In another embodiment, (Embodiment Thirteen), the invention relates to the method according to Embodiment One, wherein
Y is CH and A is CH;
R¹ together with L represent a group selected from the group consisting of ethyl, propyl, isopropyl, isobutyl, cyclopropylmethyl, cyclobutylmethyl, 2,2-dimethylpropyl, 1-methylcyclopropylmethyl, 1-fluoromethylcyclopropylmethyl, 1-cyclopropylethyl, 2-cyclopropylethyl, cyclopentyl, cyclohexyl, 2,2-difluorocyclobutylmethyl, 3,3-difluorocyclobutylmethyl, 3-(trifluoromethyl)cyclobutylmethyl, and 3,3,3-trifluoro-2-methyl-propyl;
R² is methoxy;
R³, R⁴ and R⁵ are each H;
R⁶ is selected from the group consisting of H, methyl, and methoxy; and
R⁷ is H;
or a pharmaceutically acceptable salt thereof.

In another embodiment, (Embodiment Fourteen), the invention relates to the method according to Embodiment One, wherein
R³ and R⁵ together with the atoms to which they are attached join to form a 3- to 9-membered bicyclic ring, wherein said 3- to 9-membered bicyclic ring may optionally contain one to two heteroatoms independently selected from the group consisting of N and O,
or a pharmaceutically acceptable salt thereof.

### BRIEF DESCRIPTION OF THE FIGURES

FIG. 1 shows the study design of a clinical trial for demonstrating the use of an exemplary compound of the invention (Compound 17) for treating FSGS.

### DETAILED DESCRIPTION OF THE INVENTION

### Abbreviations

ACE Angiotensin Converting Enzyme
AE Adverse Event
AESI Adverse Event of Special Interest
AHR Aryl Hydrocarbon Receptor
ALT Alanine Aminotransferase
ANOVA Analysis of Variance
ARB Angiotensin II Receptor Blockers
AST Aspartate Aminotransferase
AUC Area under the Plasma Concentration Curve
AUC_{0-∞} Area under the Plasma Concentration Curve from 0 to ∞
AUCₜ₁₋ₜ₂ Area under the Plasma Concentration Curve from t1 to t2
AUC_{t1-t2,ss} Area under the Plasma Concentration Curve from t1 to t2 at steady state
BI Boehringer Ingelheim
BMI Body Mass Index
CA Competent Authority
CAR Constitutive Androstane Receptor
CKD Chronic Kidney Disease
CKD-EPI Chronic Kidney Disease-Epidemiology Collaboration
Cₘₐₓ Maximum Plasma Concentration
C_{max,ss} Maximum Plasma Concentration at steady state
C_{pre,ss} Pre-dose Plasma Concentration at steady state
C_{trough,ss} Trough Plasma Concentration at steady state
COVID-19 Coronavirus Disease 2019
CRA Clinical Research Associate
CRO Contract Research Organization
CSA Cyclosporine
CT Manager Clinical Trial Manager
CTL Clinical Trial Leader
CYP3A4 Cytochrome P450 3A4
DCT Decentralized Clinical Trial
DDI Drug-Drug Interaction
DEX Dexamethasone
DILI Drug Induced Liver Injury
DMC Data Monitoring Committee
EC Ethics Committee
ECG Electrocardiogram
eCRF Electronic Case Report Form
EDC Electronic Data Capture
eGFR Estimated Glomerular Filtration Rate
EoS End of Study (corresponds with End of Trial)
EoT End of Treatment
ES Entered Set
ESKD End Stage Kidney Disease
EudraCT European Clinical Trials Database
FAS Full Analysis Set
FDA Food and Drug Administration
FSGS Focal Segmental Glomerulosclerosis
FUP1 Follow-up Visit #1
GCP Good Clinical Practice
gCV Geometric Coefficient of Variation
GGT Gamma-Glutamyl Transferase
GI Gastrointestinal
gMean Geometric Mean
HA Health Authority
HR Heart Rate
IB Investigator's Brochure
ICE Intercurrent Event
ICF Informed Consent Form
ICH International Council on Harmonisation
IEC Independent Ethics Committee
IgA Immunoglobulin A
IRB Institutional Review Board
IRT Interactive Response Technology
ISF Investigator Site File
DILI Drug Induced Liver Injury
DMC Data Monitoring Committee
EC Ethics Committee
ECG Electrocardiogram
eCRF Electronic Case Report Form
EDC Electronic Data Capture
eGFR Estimated Glomerular Filtration Rate
EoS End of Study (corresponds with End of Trial)
EoT End of Treatment
ES Entered Set
ESKD End Stage Kidney Disease
EudraCT European Clinical Trials Database
FAS Full Analysis Set
FDA Food and Drug Administration
FSGS Focal Segmental Glomerulosclerosis
FUP1 Follow-up Visit #1
GCP Good Clinical Practice
gCV Geometric Coefficient of Variation
GGT Gamma-Glutamyl Transferase
GI Gastrointestinal
gMean Geometric Mean
HA Health Authority
HR Heart Rate
IB Investigator's Brochure
ICE Intercurrent Event
ICF Informed Consent Form
ICH International Council on Harmonisation
IEC Independent Ethics Committee
IgA Immunoglobulin A
IRB Institutional Review Board
IRT Interactive Response Technology
ISF Investigator Site File
K-EDTA Potassium Ethylenediaminetetraacetic Acid
LOCS Ill Lens Opacities Classification System Ill
LPLT Last Patient Last Treatment
MATE1 Multidrug and Toxin Extrusion 1
MATE2 Multidrug and Toxin Extrusion 2
MedDRA Medical Dictionary for Drug Regulatory Activities
MMF Mycophenolate Mofetil
MRN Mobile Research Nurse
ms Milliseconds
mRNA Messenger RNA
NEPTUNE Nephrotic Syndrome Study Network
NFAT Nuclear Factor of Activated T-cells
NOAEL No Observed Adverse Effect level
OCT2 Organic Cation Transporter 2
P-gp Permeability Glycoprotein
PD Pharmacodynamics
PE Physical Exam
PK Pharmacokinetics
p.o. per os (oral)
q.d. quaque die (once a day)
QT Time between start of the Q-wave and end of the T-wave in an electrocardiogram
QTc QT interval corrected for heart rate
QTcF QT interval corrected for heart rate using the method of Fridericia
RA Regulatory Authority
SAE Serious Adverse Event
SARS-CoV-2 Severe Acute Respiratory Syndrome Coronavirus 2
sFSGS Secondary Focal Segmental Glomerulosclerosis
SGLT2 Sodium-Glucose Cotransporter-2
SOP Standard Operating Procedure
SULT Sulfotransferase
SUSAR Suspected Unexpected Serious Adverse Reactions
TS Treated set
TRPC6 Transient Receptor Potential Cation subfamily C Member 6
TSAP Trial Statistical Analysis Plan
t_{1/2} Terminal half-Life of the analyte
tₘₐₓ Time to Maximum Plasma Concentration
UACR Urine Albumin Creatinine Ratio
UGT Uridine Diphosphate Glucuronosyl Transferase
ULN Upper Level of Normal
UNephCR Urine Nephrin Creatinine Ratio
UPCR Urine Protein-Creatinine Ratio
UPNR Urine Podocin Nephrin Ratio
UPodCR Urine Podocin Creatinine Ratio
WOCBP Woman of Childbearing Potential

As noted above, the invention relates to methods for treating an FSGS patient, comprising administering to the patient a pharmaceutically effective amount of a compound of formula (I) as defined above, or a pharmaceutically acceptable salt thereof.

The method of the invention also relates to a compound of formula (I) as defined above, and the pharmaceutically acceptable salts therefore, for use in the treatment of a patient with FSGS.

As used herein, the term “compound of the invention” or “compounds of the invention” refers to any compound embraced by the compound of formula (I) as defined above and in Table 1 below, and the pharmaceutically acceptable salts thereof.

In one embodiment, invention relates to methods for reducing the level of proteinuria in an FSGS patient, comprising administering to the patient a pharmaceutically effective amount of a compound of the invention.

In another embodiment, the invention relates to methods for reducing the level of proteinuria in an FSGS patient, wherein the method provides at least 25% reduction in 24-hour urine protein-creatinine ratio (UPCR) relative to baseline at week 12 in the patient.

In another embodiment, invention relates to methods for preserving renal function in an FSGS patient, comprising administering to the patient a pharmaceutically effective amount of a compound of the invention.

In another embodiment, the invention relates to methods for preserving renal function in an FSGS patient, wherein the method preserves the estimated glomerular filtration rate (eGFR) in the patient. In another embodiment, the eGFR is based on serum cystatin C values.

In another embodiment, invention relates to methods for reducing the level of proteinuria and preserving renal function in an FSGS patient, comprising administering to the patient a pharmaceutically effective amount of a compound of the invention.

In one embodiment, the invention relates to a compound of the invention for use in the treatment of a patient with FSGS.

In another embodiment, invention relates to a compound of the invention for use in reducing the level of proteinuria in an FSGS patient.

In another embodiment, the invention relates to a compound of the invention for use in reducing the level of proteinuria in an FSGS patient, wherein the method provides at least 25% reduction in 24-hour urine protein-creatinine ratio (UPCR) relative to baseline at week 12 in the patient.

In another embodiment, invention relates to a compound of the invention for use in preserving renal function in an FSGS patient. In another embodiment, the preservation of renal function is an improvement in the eGFR of the patient from visit 2 (before first dose) to week 12 and/or 13 using the Chronic Kidney Disease-Epidemiology Collaboration (CKD-EPI) formula based on serum cystatin C for efficacy analysis.

In another embodiment, invention relates to a compound of the invention for use in reducing the level of proteinuria and preserving renal function in an FSGS patient, comprising administering to the patient a pharmaceutically effective amount of a compound of the invention.

Table 1 shows specific compounds of the invention which may be used according to the methods of the invention.

TABLE 1

| Cpd No. | Structure | Compound Name |
|---|---|---|
| 1 | 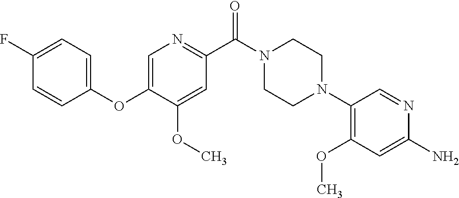 [SMILES: COc1cc(C(=O)N2CCN(c3cnc(N)cc3OC)CC2)ncc1Oc1ccc(F)cc1] | [4-(6-Amino-4-methoxy-pyridin-3- yl)-piperazin-1-yl]-[5-(4-fluoro- phenoxy)-4-methoxy-pyridin-2-yl]- methanone |
| 2 | 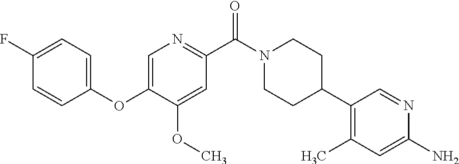 [SMILES: COc1cc(C(=O)N2CCC(c3cnc(N)cc3C)CC2)ncc1Oc1ccc(F)cc1] | (6-Amino-4-methyl-3′,4′,5′,6′- tetrahydro-2′H-[3,4′]bipyridinyl-1′-yl)- [5-(4-fluoro-phenoxy)-4-methoxy- pyridin-2-yl]-methanone |
| 3 | 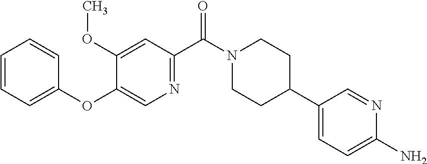 [SMILES: COc1cc(C(=O)N2CCC(c3ccc(N)nc3)CC2)ncc1Oc1ccccc1] | (6-Amino-3′,4′,5′,6′-tetrahydro-2′H- [3,4′]bipyridinyl-1′-yl)-(4-methoxy-5- phenoxy-pyridin-2-yl)-methanone |
| 4 | 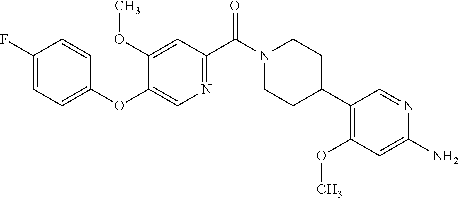 [SMILES: COc1cc(C(=O)N2CCC(c3cnc(N)cc3OC)CC2)ncc1Oc1ccc(F)cc1] | (6-Amino-4-methoxy-3′,4′,5′,6′- tetrahydro-2′H-[3,4′]bipyridinyl-1′-yl)- [5-(4-fluoro-phenoxy)-4-methoxy- pyridin-2-yl]-methanone |
| 5 | 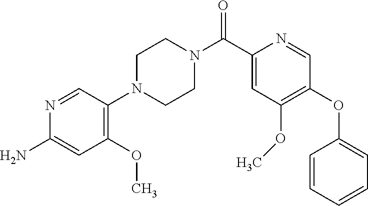 [SMILES: COc1cc(C(=O)N2CCN(c3cnc(N)cc3OC)CC2)ncc1Oc1ccccc1] | [4-(6-Amino-4-methoxy-pyridin-3- yl)-piperazin-1-yl]-(4-methoxy-5- phenoxy-pyridin-2-yl)-methanone |
| 6 | 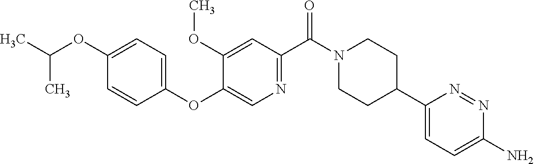 [SMILES: COc1cc(C(=O)N2CCC(c3ccc(N)nn3)CC2)ncc1Oc1ccc(OC(C)C)cc1] | [4-(6-Amino-pyridazin-3-yl)- piperidin-1-yl]-[5-(4-isopropoxy- phenoxy)-4-methoxy-pyridin-2-yl]- methanone |
| 7 | 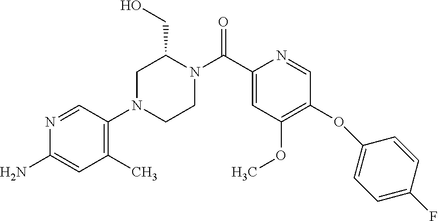 [SMILES: COc1cc(C(=O)N2CCN(c3cnc(N)cc3C)C[C@@H]2CO)ncc1Oc1ccc(F)cc1] | [(R)-4-(6-Amino-4-methyl-pyridin-3- yl)-2-hydroxymethyl-piperazin-1-yl]- [5-(4-fluoro-phenoxy)-4-methoxy- pyridin-2-yl]-methanone |
| 8 | 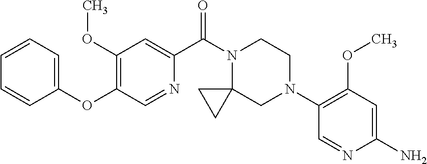 [SMILES: COc1cc(C(=O)N2CCN(c3cnc(N)cc3OC)CC23CC3)ncc1Oc1ccccc1] | [7-(6-Amino-4-methoxy-pyridin-3- yl)-4,7-diaza-spiro[2.5]oct-4-yl]-(4- methoxy-5-phenoxy-pyridin-2-yl)- methanone |
| 9 | 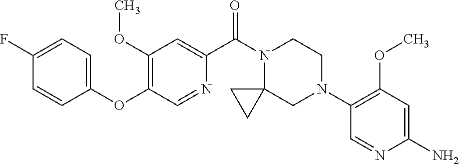 [SMILES: COc1cc(C(=O)N2CCN(c3cnc(N)cc3OC)CC23CC3)ncc1Oc1ccc(F)cc1] | [7-(6-Amino-4-methoxy-pyridin-3- yl)-4,7-diaza-spiro[2.5]oct-4-yl]-[5- (4-fluoro-phenoxy)-4-methoxy- pyridin-2-yl]-methanone |
| 10 | 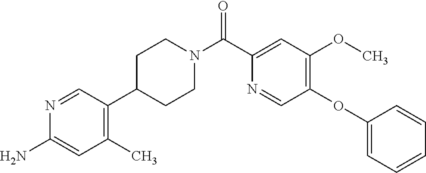 [SMILES: COc1cc(C(=O)N2CCC(c3cnc(N)cc3C)CC2)ncc1Oc1ccccc1] | (6-Amino-4-methyl-3′,4′,5′,6′- tetrahydro-2′H-[3,4′]bipyridinyl-1′-yl)- (4-methoxy-5-phenoxy-pyridin-2-yl)- methanone |
| 11 | 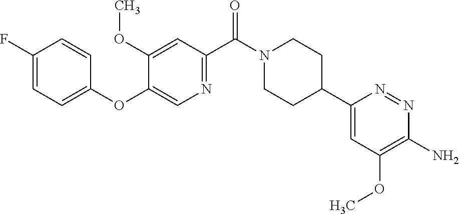 [SMILES: COc1cc(C(=O)N2CCC(c3cc(OC)c(N)nn3)CC2)ncc1Oc1ccc(F)cc1] | [4-(6-Amino-5-methoxy-pyridazin-3- yl)-piperidin-1-yl]-[5-(4-fluoro- phenoxy)-4-methoxy-pyridin-2-yl]- methanone |
| 12 | 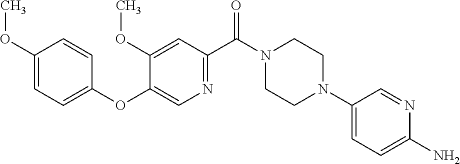 [SMILES: COc1ccc(Oc2cnc(C(=O)N3CCN(c4ccc(N)nc4)CC3)cc2OC)cc1] | [4-(6-Amino-pyridin-3-yl)-piperazin- 1-yl]-[4-methoxy-5-(4-methoxy- phenoxy)-pyridin-2-yl]-methanone |
| 13 | 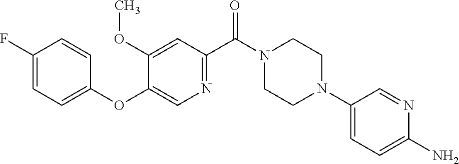 [SMILES: COc1cc(C(=O)N2CCN(c3ccc(N)nc3)CC2)ncc1Oc1ccc(F)cc1] | [4-(6-Amino-pyridin-3-yl)-piperazin- 1-yl]-[5-(4-fluoro-phenoxy)-4- methoxy-pyridin-2-yl]-methanone |
| 14 | 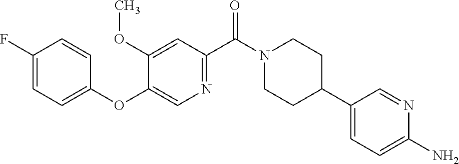 [SMILES: COc1cc(C(=O)N2CCC(c3ccc(N)nc3)CC2)ncc1Oc1ccc(F)cc1] | (6-Amino-3′,4′,5′,6′-tetrahydro-2′H- [3,4′]bipyridinyl-1′-yl)-[5-(4-fluoro- phenoxy)-4-methoxy-pyridin-2-yl]- methanone |
| 15 | 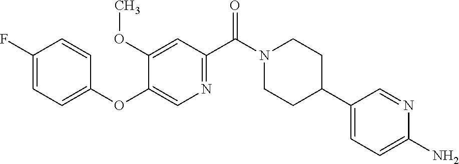 [SMILES: COc1cc(C(=O)N2CCC(c3ccc(N)nc3)CC2)ncc1Oc1ccc(F)cc1] | [4-(6-Amino-pyridin-3-yl)-piperazin- 1-yl]-(4-methoxy-5-phenoxy-pyridin- 2-yl)-methanone |
| 16 | 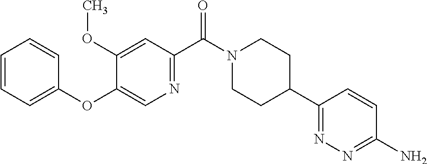 [SMILES: COc1cc(C(=O)N2CCC(c3ccc(N)nn3)CC2)ncc1Oc1ccccc1] | [4-(6-Amino-pyridazin-3-yl)- piperidin-1-yl]-(4-methoxy-5- phenoxy-pyridin-2-yl)-methanone |
| 17 | 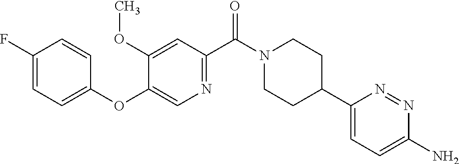 [SMILES: COc1cc(C(=O)N2CCC(c3ccc(N)nn3)CC2)ncc1Oc1ccc(F)cc1] | [4-(6-Amino-pyridazin-3-yl)- piperidin-1-yl]-[5-(4-fluoro-phenoxy)- 4-methoxy-pyridin-2-yl]-methanone |
| 18 | 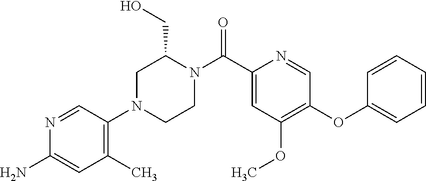 [SMILES: COc1cc(C(=O)N2CCN(c3cnc(N)cc3C)C[C@@H]2CO)ncc1Oc1ccccc1] | [(R)-4-(6-Amino-4-methyl-pyridin-3- yl)-2-hydroxymethyl-piperazin-1-yl]- (4-methoxy-5-phenoxy-pyridin-2-yl)- methanone |
| 19 | 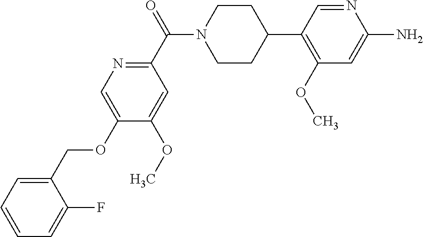 [SMILES: COc1cc(C(=O)N2CCC(c3cnc(N)cc3OC)CC2)ncc1OCc1ccccc1F] | (6-Amino-4-methoxy-3′,4′,5′,6′- tetrahydro-2′H-[3,4′]bipyridinyl-1′-yl)- [5-(2-fluoro-benzyloxy)-4-methoxy- pyridin-2-yl]-methanone |
| 20 | 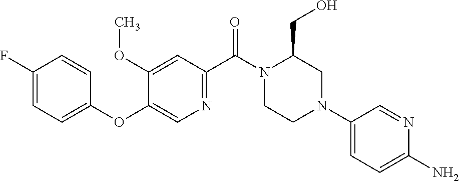 [SMILES: COc1cc(C(=O)N2CCN(c3ccc(N)nc3)C[C@@H]2CO)ncc1Oc1ccc(F)cc1] | [(R)-4-(6-Amino-pyridin-3-yl)-2- hydroxymethyl-piperazin-1-yl]-[5-(4- fluoro-phenoxy)-4-methoxy-pyridin- 2-yl]-methanone |
| 21 | 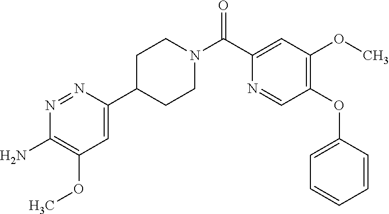 [SMILES: COc1cc(C(=O)N2CCC(c3cc(OC)c(N)nn3)CC2)ncc1Oc1ccccc1] | [4-(6-Amino-5-methoxy-pyridazin-3- yl)-piperidin-1-yl]-(4-methoxy-5- phenoxy-pyridin-2-yl)-methanone |
| 22 | 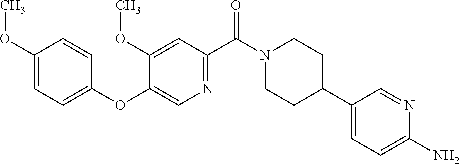 [SMILES: COc1ccc(Oc2cnc(C(=O)N3CCC(c4ccc(N)nc4)CC3)cc2OC)cc1] | (6-Amino-3′,4′,5′,6′-tetrahydro-2′H- [3,4′]bipyridinyl-1′-yl)-[4-methoxy-5- (4-methoxy-phenoxy)-pyridin-2-yl]- methanone |
| 23 | 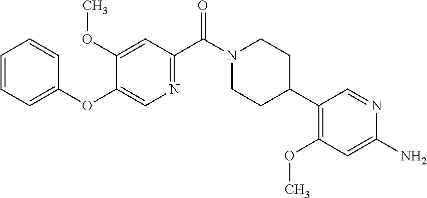 [SMILES: COc1cc(C(=O)N2CCC(c3cnc(N)cc3OC)CC2)ncc1Oc1ccccc1] | (6-Amino-4-methoxy-3′,4′,5′,6′- tetrahydro-2′H-[3,4′]bipyridinyl-1′-yl)- (4-methoxy-5-phenoxy-pyridin-2-yl)- methanone |
| 24 | 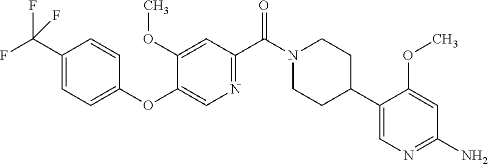 [SMILES: COc1cc(C(=O)N2CCC(c3cnc(N)cc3OC)CC2)ncc1Oc1ccc(C(F)(F)F)cc1] | (6-Amino-4-methoxy-3′,4′,5′,6′- tetrahydro-2′H-[3,4′]bipyridinyl-1′-yl)- [4-methoxy-5-(4-trifluoromethyl- phenoxy)-pyridin-2-yl]-methanone |
| 25 | 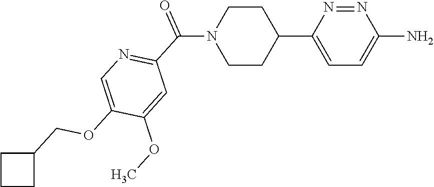 [SMILES: COc1cc(C(=O)N2CCC(c3ccc(N)nn3)CC2)ncc1OCC1CCC1] | [4-(6-Amino-pyridazin-3-yl)- piperidin-1-yl]-(5-cyclobutylmethoxy- 4-methoxy-pyridin-2-yl)-methanone |
| 26 | 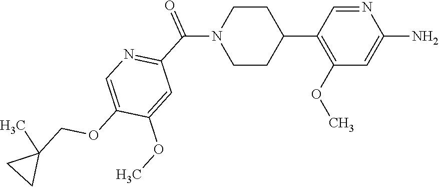 [SMILES: COc1cc(C(=O)N2CCC(c3cnc(N)cc3OC)CC2)ncc1OCC1(C)CC1] | (6-Amino-4-methoxy-3′,4′,5′,6′- tetrahydro-2′H-[3,4′]bipyridinyl-1′-yl)- [4-methoxy-5-(1-methyl- cyclopropylmethoxy)-pyridin-2-yl]- methanone |
| 27 | 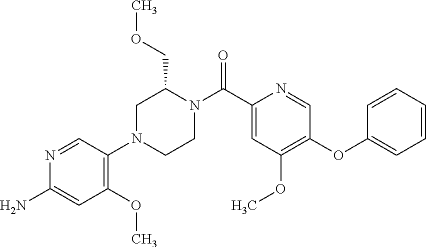 [SMILES: COC[C@H]1CN(c2cnc(N)cc2OC)CCN1C(=O)c1cc(OC)c(Oc2ccccc2)cn1] | [(R)-4-(6-Amino-4-methoxy-pyridin- 3-yl)-2-methoxymethyl-piperazin-1- yl]-(4-methoxy-5-phenoxy-pyridin-2- yl)-methanone |
| 28 | 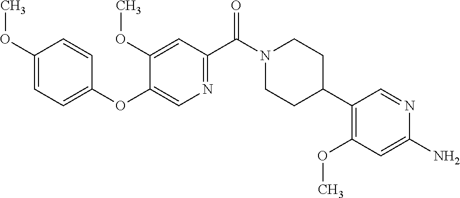 [SMILES: COc1ccc(Oc2cnc(C(=O)N3CCC(c4cnc(N)cc4OC)CC3)cc2OC)cc1] | (6-Amino-4-methoxy-3′,4′,5′,6′- tetrahydro-2′H-[3,4′]bipyridinyl-1′-yl)- [4-methoxy-5-(4-methoxy-phenoxy)- pyridin-2-yl]-methanone |
| 29 | 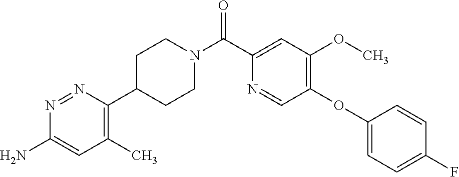 [SMILES: COc1cc(C(=O)N2CCC(c3nnc(N)cc3C)CC2)ncc1Oc1ccc(F)cc1] | [4-(6-Amino-4-methyl-pyridazin-3- yl)-piperidin-1-yl]-[5-(4-fluoro- phenoxy)-4-methoxy-pyridin-2-yl]- methanone |
| 30 | 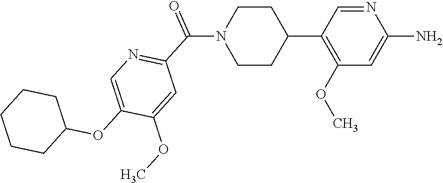 [SMILES: COc1cc(C(=O)N2CCC(c3cnc(N)cc3OC)CC2)ncc1OC1CCCCC1] | (6-Amino-4-methoxy-3′,4′,5′,6′- tetrahydro-2′H-[3,4′]bipyridinyl-1′-yl)- (5-cyclohexyloxy-4-methoxy-pyridin- 2-yl)-methanone |
| 31 | 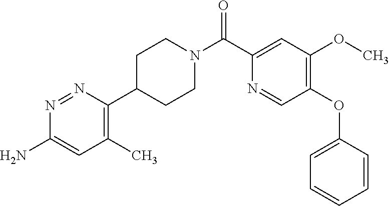 [SMILES: COc1cc(C(=O)N2CCC(c3nnc(N)cc3C)CC2)ncc1Oc1ccccc1] | [4-(6-Amino-4-methyl-pyridazin-3- yl)-piperidin-1-yl]-(4-methoxy-5- phenoxy-pyridin-2-yl)-methanone |
| 32 | 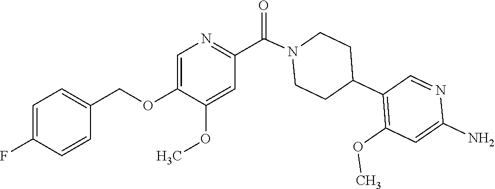 [SMILES: COc1cc(C(=O)N2CCC(c3cnc(N)cc3OC)CC2)ncc1OCc1ccc(F)cc1] | (6-Amino-4-methoxy-3′,4′,5′,6′- tetrahydro-2′H-[3,4′]bipyridinyl-1′-yl)- [5-(4-fluoro-benzyloxy)-4-methoxy- pyridin-2-yl]-methanone |
| 33 | 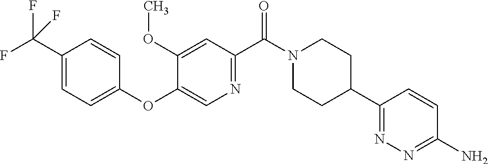 [SMILES: COc1cc(C(=O)N2CCC(c3ccc(N)nn3)CC2)ncc1Oc1ccc(C(F)(F)F)cc1] | [4-(6-Amino-pyridazin-3-yl)- piperidin-1-yl]-[4-methoxy-5-(4- trifluoromethyl-phenoxy)-pyridin-2- yl]-methanone |
| 34 | 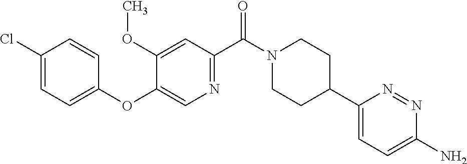 [SMILES: COc1cc(C(=O)N2CCC(c3ccc(N)nn3)CC2)ncc1Oc1ccc(Cl)cc1] | [4-(6-Amino-pyridazin-3-yl)- piperidin-1-yl]-[5-(4-chloro- phenoxy)-4-methoxy-pyridin-2-yl]- methanone |
| 35 | 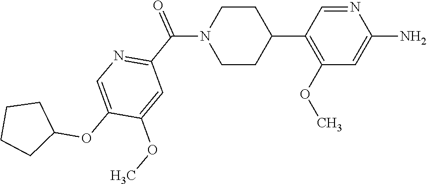 [SMILES: COc1cc(C(=O)N2CCC(c3cnc(N)cc3OC)CC2)ncc1OC1CCCC1] | (6-Amino-4-methoxy-3′,4′,5′,6′- tetrahydro-2′H-[3,4′]bipyridinyl-1′-yl)- (5-cyclopentyloxy-4-methoxy- pyridin-2-yl)-methanone |
| 36 | 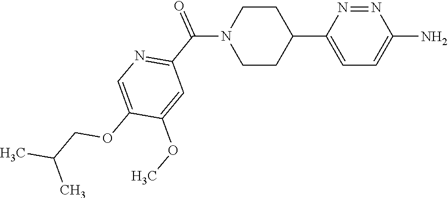 [SMILES: COc1cc(C(=O)N2CCC(c3ccc(N)nn3)CC2)ncc1OCC(C)C] | [4-(6-Amino-pyridazin-3-yl)- piperidin-1-yl]-(5-isobutoxy-4- methoxy-pyridin-2-yl)-methanone |
| 37 | 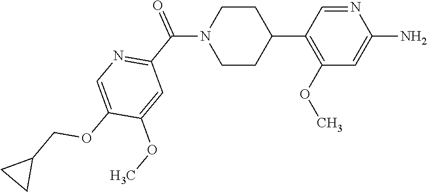 [SMILES: COc1cc(C(=O)N2CCC(c3cnc(N)cc3OC)CC2)ncc1OCC1CC1] | (6-Amino-4-methoxy-3′,4′,5′,6′- tetrahydro-2′H-[3,4′]bipyridinyl-1′-yl)- (5-cyclopropylmethoxy-4-methoxy- pyridin-2-yl)-methanone |
| 38 | 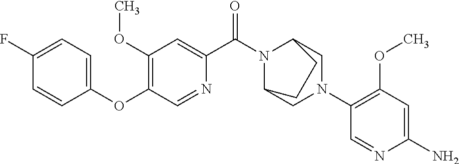 [SMILES: COc1cc(C(=O)N2C3CCC2CN(c2cnc(N)cc2OC)C3)ncc1Oc1ccc(F)cc1] | [3-(6-Amino-4-methoxy-pyridin-3-yl)- 3,8-diaza-bicyclo[3.2.1]oct-8-yl]-[5- (4-fluoro-phenoxy)-4-methoxy- pyridin-2-yl]-methanone |
| 39 | 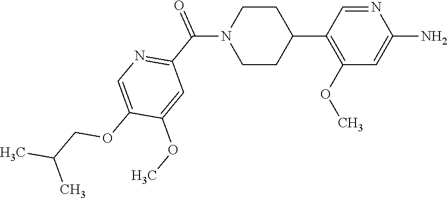 [SMILES: COc1cc(C(=O)N2CCC(c3cnc(N)cc3OC)CC2)ncc1OCC(C)C] | (6-Amino-4-methoxy-3′,4′,5′,6′- tetrahydro-2′H-[3,4′]bipyridinyl-1′-yl)- (5-isobutoxy-4-methoxy-pyridin-2-yl)- methanone |
| 40 | 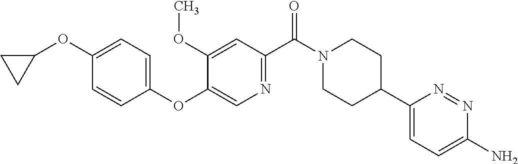 [SMILES: COc1cc(C(=O)N2CCC(c3ccc(N)nn3)CC2)ncc1Oc1ccc(OC2CC2)cc1] | [4-(6-Amino-pyridazin-3-yl)-piperidin- 1-yl]-[5-(4-cyclopropoxy-phenoxy)-4- methoxy-pyridin-2-yl]-methanone |
| 41 | 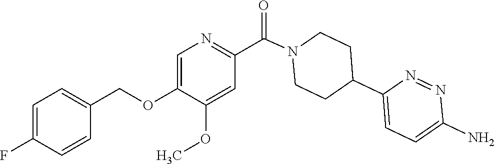 [SMILES: COc1cc(C(=O)N2CCC(c3ccc(N)nn3)CC2)ncc1OCc1ccc(F)cc1] | [4-(6-Amino-pyridazin-3-yl)-piperidin- 1-yl]-[5-(4-fluoro-benzyloxy)-4- methoxy-pyridin-2-yl]-methanone |
| 42 | 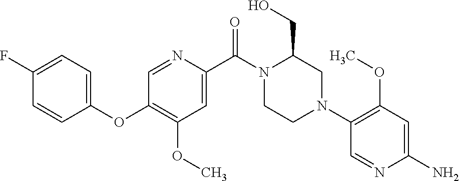 [SMILES: COc1cc(C(=O)N2CCN(c3cnc(N)cc3OC)C[C@@H]2CO)ncc1Oc1ccc(F)cc1] | [(R)-4-(6-Amino-4-methoxy-pyridin-3- yl)-2-hydroxymethyl-piperazin-1-yl]- [5-(4-fluoro-phenoxy)-4-methoxy- pyridin-2-yl]-methanone |
| 43 | 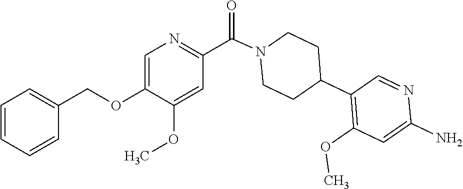 [SMILES: COc1cc(C(=O)N2CCC(c3cnc(N)cc3OC)CC2)ncc1OCc1ccccc1] | (6-Amino-4-methoxy-3′,4′,5′,6′- tetrahydro-2′H-[3,4′]bipyridinyl-1′-yl)- (5-benzyloxy-4-methoxy-pyridin-2- yl)-methanone |
| 44 | 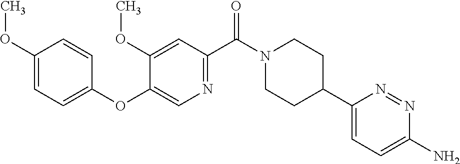 [SMILES: COc1ccc(Oc2cnc(C(=O)N3CCC(c4ccc(N)nn4)CC3)cc2OC)cc1] | [4-(6-Amino-pyridazin-3-yl)-piperidin- 1-yl]-[4-methoxy-5-(4-methoxy- phenoxy)-pyridin-2-yl]-methanone |
| 45 | 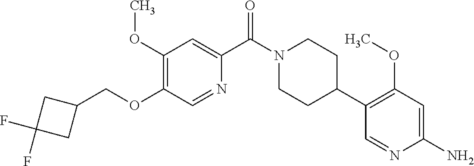 [SMILES: COc1cc(C(=O)N2CCC(c3cnc(N)cc3OC)CC2)ncc1OCC1CC(F)(F)C1] | (6-Amino-4-methoxy-3′,4′,5′,6′- tetrahydro-2′H-[3,4′]bipyridinyl-1′-yl)- [5-(3,3-difluoro-cyclobutylmethoxy)- 4-methoxy-pyridin-2-yl]-methanone |
| 46 | 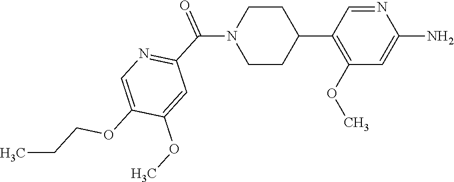 [SMILES: CCCOc1cnc(C(=O)N2CCC(c3cnc(N)cc3OC)CC2)cc1OC] | (6-Amino-4-methoxy-3′,4′,5′,6′- tetrahydro-2′H-[3,4′]bipyridinyl-1′-yl)- (4-methoxy-5-propoxy-pyridin-2-yl)- methanone |
| 47 | 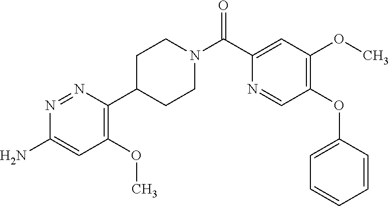 [SMILES: COc1cc(C(=O)N2CCC(c3nnc(N)cc3OC)CC2)ncc1Oc1ccccc1] | [4-(6-Amino-4-methoxy-pyridazin-3- yl)-piperidin-1-yl]-(4-methoxy-5- phenoxy-pyridin-2-yl)-methanone |
| 48 | 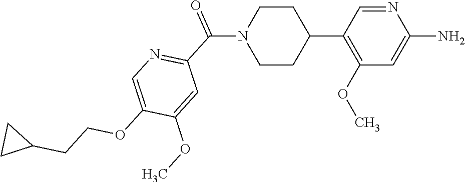 [SMILES: COc1cc(C(=O)N2CCC(c3cnc(N)cc3OC)CC2)ncc1OCCC1CC1] | (6-Amino-4-methoxy-3′,4′,5′,6′- tetrahydro-2′H-[3,4′]bipyridinyl-1′-yl)- [5-(2-cyclopropyl-ethoxy)-4-methoxy- pyridin-2-yl]-methanone |
| 49 | 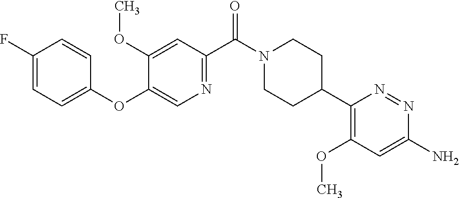 [SMILES: COc1cc(C(=O)N2CCC(c3nnc(N)cc3OC)CC2)ncc1Oc1ccc(F)cc1] | [4-(6-Amino-4-methoxy-pyridazin-3- yl)-piperidin-1-yl]-[5-(4-fluoro- phenoxy)-4-methoxy-pyridin-2-yl]- methanone |
| 50 | 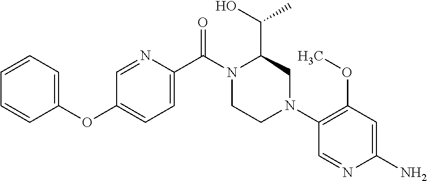 [SMILES: COc1cc(N)ncc1N1CCN(C(=O)c2ccc(Oc3ccccc3)cn2)[C@@H]([C@@H](C)O)C1] | (1R)-1-[(2R)-4-(6-amino-4- methoxypyridin-3-yl)-1-(5- phenoxypyridine-2- carbonyl)piperazin-2-yllethan-1-ol |
| 51 | 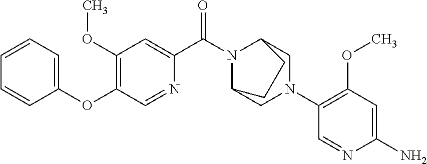 [SMILES: COc1cc(C(=O)N2C3CCC2CN(c2cnc(N)cc2OC)C3)ncc1Oc1ccccc1] | [3-(6-Amino-4-methoxy-pyridin-3-yl)- 3,8-diaza-bicyclo[3.2.1]oct-8-yl]-(4- methoxy-5-phenoxy-pyridin-2-yl)- methanone |
| 52 | 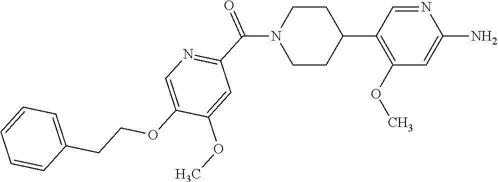 [SMILES: COc1cc(C(=O)N2CCC(c3cnc(N)cc3OC)CC2)ncc1OCCc1ccccc1] | (6-Amino-4-methoxy-3′,4′,5′,6′- tetrahydro-2′H-[3,4′]bipyridinyl-1′-yl)- (4-methoxy-5-phenethyloxy-pyridin- 2-yl)-methanone |
| 53 | 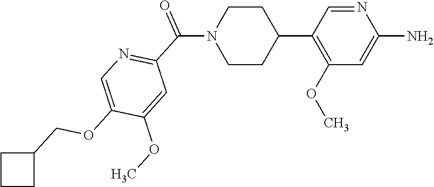 [SMILES: COc1cc(C(=O)N2CCC(c3cnc(N)cc3OC)CC2)ncc1OCC1CCC1] | (6-Amino-4-methoxy-3′,4′,5′,6′- tetrahydro-2′H-[3,4′]bipyridinyl-1′-yl)- (5-cyclobutylmethoxy-4-methoxy- pyridin-2-yl)-methanone |
| 54 | 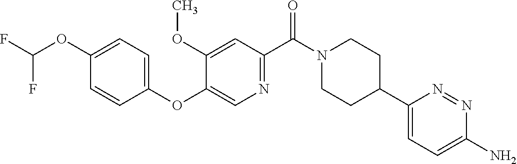 [SMILES: COc1cc(C(=O)N2CCC(c3ccc(N)nn3)CC2)ncc1Oc1ccc(OC(F)F)cc1] | [4-(6-Amino-pyridazin-3-yl)-piperidin- 1-yl]-[5-(4-difluoromethoxy-phenoxy)- 4-methoxy-pyridin-2-yl]-methanone |
| 55 | 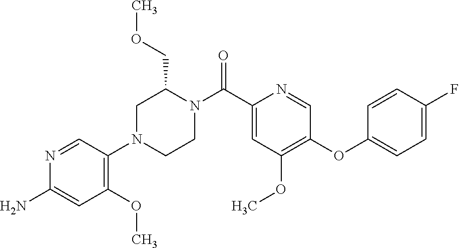 [SMILES: COC[C@H]1CN(c2cnc(N)cc2OC)CCN1C(=O)c1cc(OC)c(Oc2ccc(F)cc2)cn1] | [(R)-4-(6-Amino-4-methoxy-pyridin-3- yl)-2-methoxymethyl-piperazin-1-yl]- [5-(4-fluoro-phenoxy)-4-methoxy- pyridin-2-yl]-methanone |
| 56 | 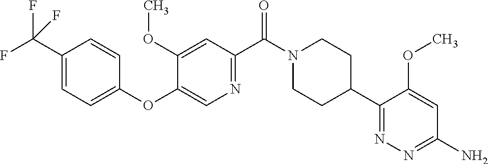 [SMILES: COc1cc(C(=O)N2CCC(c3nnc(N)cc3OC)CC2)ncc1Oc1ccc(C(F)(F)F)cc1] | [4-(6-Amino-4-methoxy-pyridazin-3- yl)-piperidin-1-yl]-[4-methoxy-5-(4- trifluoromethyl-phenoxy)-pyridin-2- yl]-methanone |
| 57 | 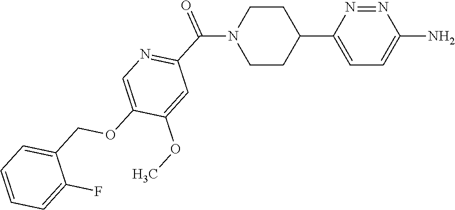 [SMILES: COc1cc(C(=O)N2CCC(c3ccc(N)nn3)CC2)ncc1OCc1ccccc1F] | [4-(6-Amino-pyridazin-3-yl)-piperidin- 1-yl]-[5-(2-fluoro-benzyloxy)-4- methoxy-pyridin-2-yl]-methanone |
| 58 | 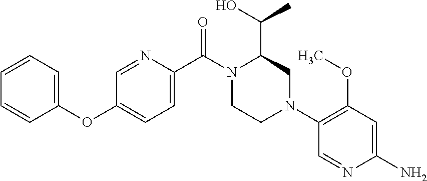 [SMILES: COc1cc(N)ncc1N1CCN(C(=O)c2ccc(Oc3ccccc3)cn2)[C@@H]([C@H](C)O)C1] | (1S)-1-[(2R)-4-(6-amino-4- methoxypyridin-3-yl)-1-(5- phenoxypyridine-2- carbonyl)piperazin-2-yllethan-1-ol |
| 59 | 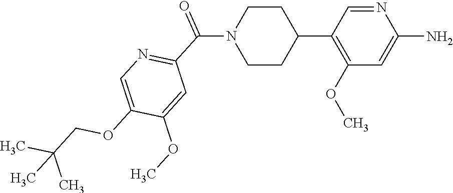 [SMILES: COc1cc(C(=O)N2CCC(c3cnc(N)cc3OC)CC2)ncc1OCC(C)(C)C] | (6-Amino-4-methoxy-3′,4′,5′,6′- tetrahydro-2′H-[3,4′]bipyridinyl-1′-yl)- [5-(2,2-dimethyl-propoxy)-4-methoxy- pyridin-2-yl]-methanone |
| 60 | 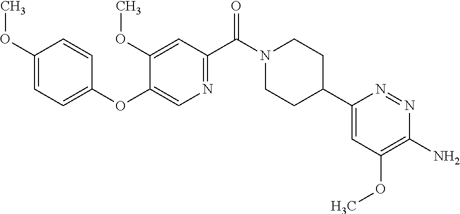 [SMILES: COc1ccc(Oc2cnc(C(=O)N3CCC(c4cc(OC)c(N)nn4)CC3)cc2OC)cc1] | [4-(6-Amino-5-methoxy-pyridazin-3- yl)-piperidin-1-yl]-[4-methoxy-5-(4- methoxy-phenoxy)-pyridin-2-yl]- methanone |
| 61 | 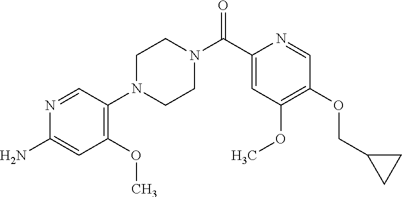 [SMILES: COc1cc(C(=O)N2CCN(c3cnc(N)cc3OC)CC2)ncc1OCC1CC1] | [4-(6-Amino-4-methoxy-pyridin-3-yl)- piperazin-1-yl]-(5- cyclopropylmethoxy-4-methoxy- pyridin-2-yl)-methanone |
| 62 | 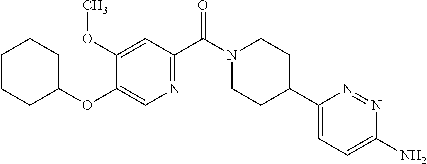 [SMILES: COc1cc(C(=O)N2CCC(c3ccc(N)nn3)CC2)ncc1OC1CCCCC1] | [4-(6-Amino-pyridazin-3-yl)-piperidin- 1-yl]-(5-cyclohexyloxy-4-methoxy- pyridin-2-yl)-methanone |
| 63 | 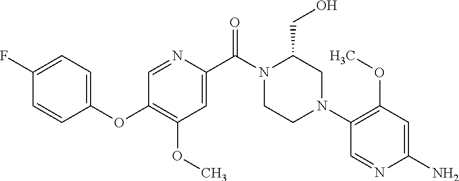 [SMILES: COc1cc(C(=O)N2CCN(c3cnc(N)cc3OC)C[C@H]2CO)ncc1Oc1ccc(F)cc1] | [(S)-4-(6-Amino-4-methoxy-pyridin-3- yl)-2-hydroxymethyl-piperazin-1-yl]- [5-(4-fluoro-phenoxy)-4-methoxy- pyridin-2-yl]-methanone |
| 64 | 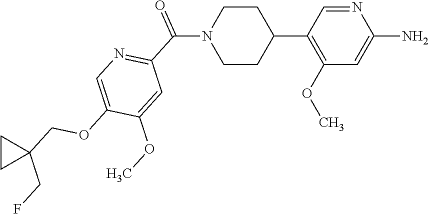 [SMILES: COc1cc(C(=O)N2CCC(c3cnc(N)cc3OC)CC2)ncc1OCC1(CF)CC1] | (6-Amino-4-methoxy-3′,4′,5′,6′- tetrahydro-2′H-[3,4′]bipyridinyl-1′-yl)- [5-(1-fluoromethyl- cyclopropylmethoxy)-4-methoxy- pyridin-2-yl]-methanone |
| 65 | 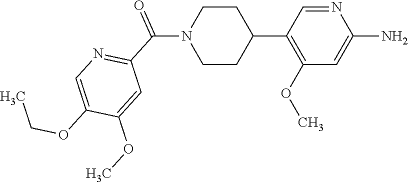 [SMILES: CCOc1cnc(C(=O)N2CCC(c3cnc(N)cc3OC)CC2)cc1OC] | (6-Amino-4-methoxy-3′,4′,5′,6′- tetrahydro-2′H-[3,4′]bipyridinyl-1′-yl)- (5-ethoxy-4-methoxy-pyridin-2-yl)- methanone |
| 66 | 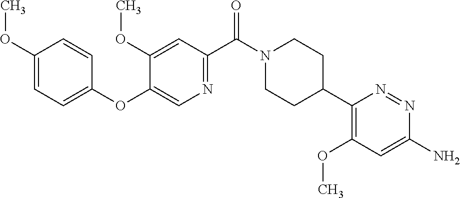 [SMILES: COc1ccc(Oc2cnc(C(=O)N3CCC(c4nnc(N)cc4OC)CC3)cc2OC)cc1] | [4-(6-Amino-4-methoxy-pyridazin-3- yl)-piperidin-1-yl]-[4-methoxy-5-(4- methoxy-phenoxy)-pyridin-2-yl]- methanone |
| 67 | 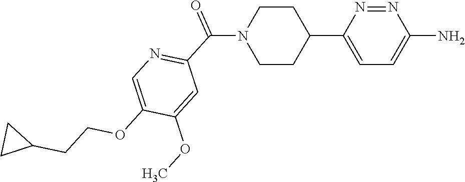 [SMILES: COc1cc(C(=O)N2CCC(c3ccc(N)nn3)CC2)ncc1OCCC1CC1] | [4-(6-Amino-pyridazin-3-yl)-piperidin- 1-yl]-[5-(2-cyclopropyl-ethoxy)-4- methoxy-pyridin-2-yl]-methanone |
| 68 | 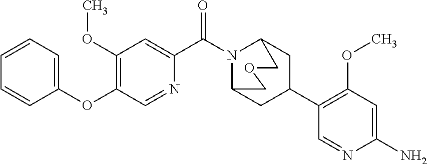 [SMILES: COc1cc(C(=O)N2C3COCC2CC(c2cnc(N)cc2OC)C3)ncc1Oc1ccccc1] | [7-(6-Amino-4-methoxy-pyridin-3-yl)- 3-oxa-9-aza-bicyclo[3.3.1]non-9-yl]- (4-methoxy-5-phenoxy-pyridin-2-yl)- methanone |
| 69 | 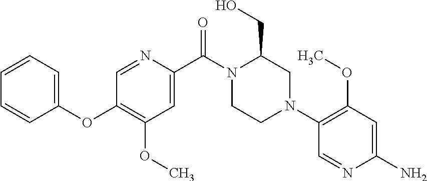 [SMILES: COc1cc(C(=O)N2CCN(c3cnc(N)cc3OC)C[C@@H]2CO)ncc1Oc1ccccc1] | [(R)-4-(6-Amino-4-methoxy-pyridin-3- yl)-2-hydroxymethyl-piperazin-1-yl]- (4-methoxy-5-phenoxy-pyridin-2-yl)- methanone |
| 70 | 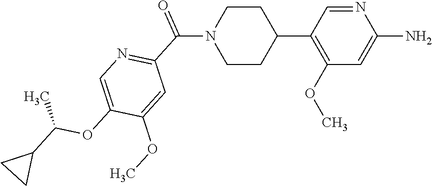 [SMILES: COc1cc(C(=O)N2CCC(c3cnc(N)cc3OC)CC2)ncc1O[C@@H](C)C1CC1] | (6-Amino-4-methoxy-3′,4′,5′,6′- tetrahydro-2′H-[3,4′]bipyridinyl-1′-yl)- [5-((S)-1-cyclopropyl-ethoxy)-4- methoxy-pyridin-2-yl]-methanone |
| 71 | 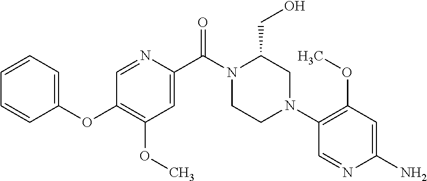 [SMILES: COc1cc(C(=O)N2CCN(c3cnc(N)cc3OC)C[C@H]2CO)ncc1Oc1ccccc1] | [(S)-4-(6-Amino-4-methoxy-pyridin-3- yl)-2-hydroxymethyl-piperazin-1-yl]- (4-methoxy-5-phenoxy-pyridin-2-yl)- methanone |
| 72 | 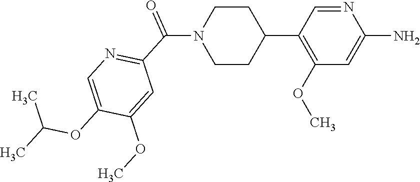 [SMILES: COc1cc(C(=O)N2CCC(c3cnc(N)cc3OC)CC2)ncc1OC(C)C] | (6-Amino-4-methoxy-3′,4′,5′,6′- tetrahydro-2′H-[3,4′]bipyridinyl-1′-yl)- (5-isopropoxy-4-methoxy-pyridin-2- yl)-methanone |
| 73 | 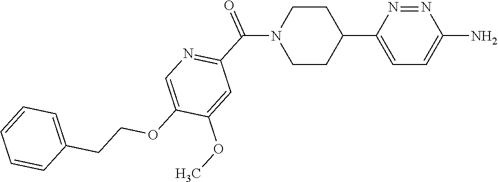 [SMILES: COc1cc(C(=O)N2CCC(c3ccc(N)nn3)CC2)ncc1OCCc1ccccc1] | [4-(6-Amino-pyridazin-3-yl)-piperidin- 1-yl]-(4-methoxy-5-phenethyloxy- pyridin-2-yl)-methanone |
| 74 | 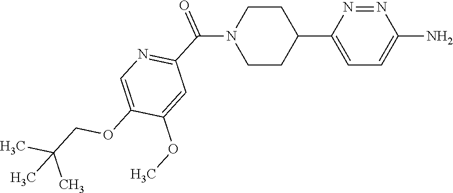 [SMILES: COc1cc(C(=O)N2CCC(c3ccc(N)nn3)CC2)ncc1OCC(C)(C)C] | [4-(6-Amino-pyridazin-3-yl)-piperidin- 1-yl]-[5-(2,2-dimethyl-propoxy)-4- methoxy-pyridin-2-yl]-methanone |
| 75 | 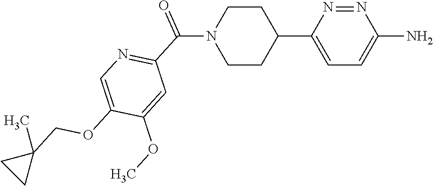 [SMILES: COc1cc(C(=O)N2CCC(c3ccc(N)nn3)CC2)ncc1OCC1(C)CC1] | [4-(6-Amino-pyridazin-3-yl)-piperidin- 1-yl]-[4-methoxy-5-(1-methyl- cyclopropylmethoxy)-pyridin-2-yl]- methanone |
| 76 | 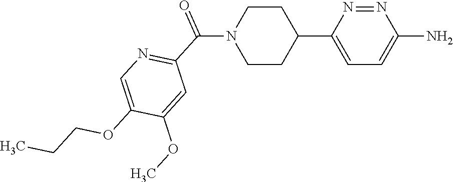 [SMILES: CCCOc1cnc(C(=O)N2CCC(c3ccc(N)nn3)CC2)cc1OC] | [4-(6-Amino-pyridazin-3-yl)-piperidin- 1-yl]-(4-methoxy-5-propoxy-pyridin-2- yl)-methanone |
| 77 | 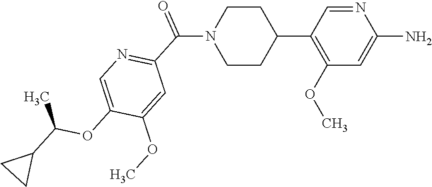 [SMILES: COc1cc(C(=O)N2CCC(c3cnc(N)cc3OC)CC2)ncc1O[C@H](C)C1CC1] | (6-Amino-4-methoxy-3′,4′,5′,6′- tetrahydro-2′H-[3,4′]bipyridinyl-1′-yl)- [5-((R)-1-cyclopropyl-ethoxy)-4- methoxy-pyridin-2-yl]-methanone |
| 78 | 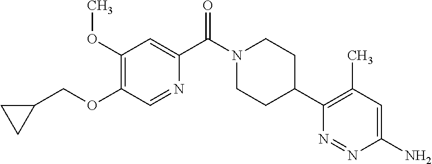 [SMILES: COc1cc(C(=O)N2CCC(c3nnc(N)cc3C)CC2)ncc1OCC1CC1] | [4-(6-Amino-4-methyl-pyridazin-3-yl)- piperidin-1-yl]-(5- cyclopropylmethoxy-4-methoxy- pyridin-2-yl)-methanone |
| 79 | 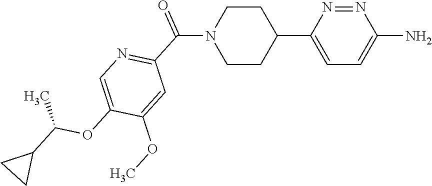 [SMILES: COc1cc(C(=O)N2CCC(c3ccc(N)nn3)CC2)ncc1O[C@@H](C)C1CC1] | [4-(6-Amino-pyridazin-3-yl)-piperidin- 1-yl]-[5-((S)-1-cyclopropyl-ethoxy)-4- methoxy-pyridin-2-yl]-methanone |
| 80 | 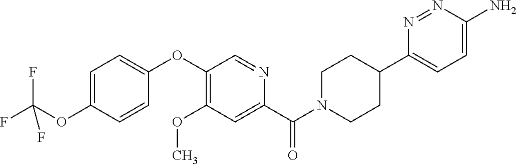 [SMILES: COc1cc(C(=O)N2CCC(c3ccc(N)nn3)CC2)ncc1Oc1ccc(OC(F)(F)F)cc1] | [4-(6-Amino-pyridazin-3-yl)-piperidin- 1-yl]-[4-methoxy-5-(4- trifluoromethoxy-phenoxy)-pyridin-2- yl]-methanone |
| 81 | 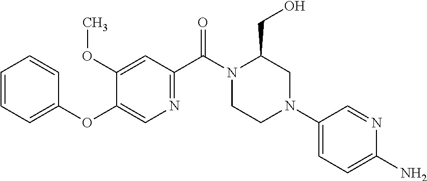 [SMILES: COc1cc(C(=O)N2CCN(c3ccc(N)nc3)C[C@@H]2CO)ncc1Oc1ccccc1] | [(R)-4-(6-Amino-pyridin-3-yl)-2- hydroxymethyl-piperazin-1-yl]-(4- methoxy-5-phenoxy-pyridin-2-yl)- methanone |
| 82 | 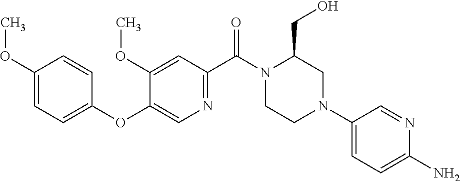 [SMILES: COc1ccc(Oc2cnc(C(=O)N3CCN(c4ccc(N)nc4)C[C@@H]3CO)cc2OC)cc1] | [(R)-4-(6-Amino-pyridin-3-yl)-2- hydroxymethyl-piperazin-1-yl]-[4- methoxy-5-(4-methoxy-phenoxy)- pyridin-2-yl]-methanone |
| 83 | 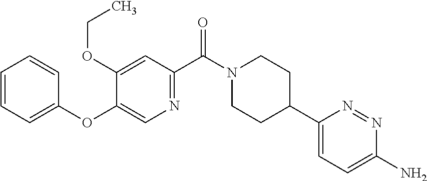 [SMILES: CCOc1cc(C(=O)N2CCC(c3ccc(N)nn3)CC2)ncc1Oc1ccccc1] | [4-(6-Amino-pyridazin-3-yl)-piperidin- 1-yl]-[5-(phenoxy)-4-ethoxy-pyridin-2- yl]-methanone |
| 84 | 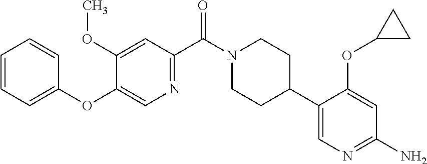 [SMILES: COc1cc(C(=O)N2CCC(c3cnc(N)cc3OC3CC3)CC2)ncc1Oc1ccccc1] | (6-Amino-4-cyclopropoxy-3′,4′,5′,6′- tetrahydro-2′H-[3,4′]bipyridinyl-1′-yl)- [5-(phenoxy)-4-methoxy-pyridin-2-yl]- methanone |
| 85 | 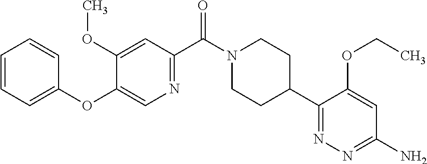 [SMILES: CCOc1cc(N)nnc1C1CCN(C(=O)c2cc(OC)c(Oc3ccccc3)cn2)CC1] | [4-(6-Amino-4-ethoxy-pyridazin-3-yl)- piperidin-1-yl]-[4-methoxy-5- (phenoxy)-pyridin-2-yl]-methanone |
| 86 | 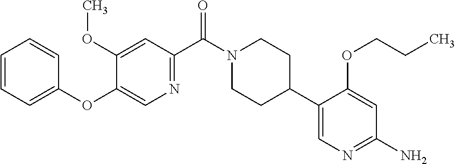 [SMILES: CCCOc1cc(N)ncc1C1CCN(C(=O)c2cc(OC)c(Oc3ccccc3)cn2)CC1] | (6-Amino-4-propoxy-3′,4′,5′,6′- tetrahydro-2′H-[3,4′]bipyridinyl-1′-yl)- [5-(phenoxy)-4-methoxy-pyridin-2-yl]- methanone |
| 87 | 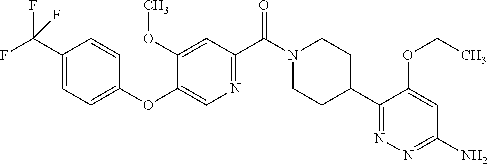 [SMILES: CCOc1cc(N)nnc1C1CCN(C(=O)c2cc(OC)c(Oc3ccc(C(F)(F)F)cc3)cn2)CC1] | (6-Amino-4-ethoxy-3′,4′,5′,6′- tetrahydro-2′H-[3,4′]bipyridinyl-1′-yl)- [5-(4-trifluoromethyl-phenoxy)-4- methoxy-pyridin-2-yl]-methanone |
| 88 | 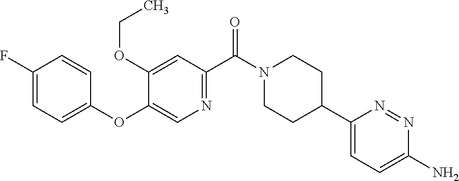 [SMILES: CCOc1cc(C(=O)N2CCC(c3ccc(N)nn3)CC2)ncc1Oc1ccc(F)cc1] | [4-(6-Amino-pyridazin-3-yl)-piperidin- 1-yl]-[5-(4-fluoro-phenoxy)-4-ethoxy- pyridin-2-yl]-methanone |
| 89 | 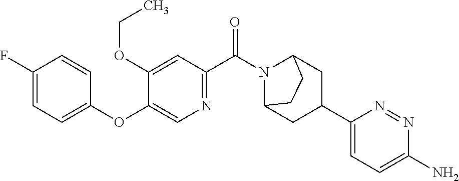 [SMILES: CCOc1cc(C(=O)N2C3CCC2CC(c2ccc(N)nn2)C3)ncc1Oc1ccc(F)cc1] | [3-(6-Amino-pyridazin-3-yl)-8-aza- bicyclo[3.2.1]oct-8-yl]-[4-ethoxy-5-(4- fluoro-phenoxy)-pyridin-2-yl]- methanone |
| 90 | 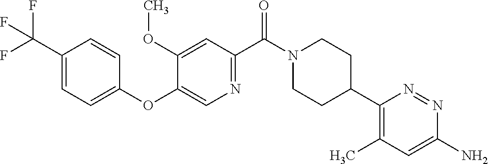 [SMILES: COc1cc(C(=O)N2CCC(c3nnc(N)cc3C)CC2)ncc1Oc1ccc(C(F)(F)F)cc1] | 6-(1-{4-Methoxy-5-[4- (trifluoromethyl)phenoxy]pyridine-2- carbonyl}piperidin-4-yl)-5- methylpyridazin-3-amine |
| 91 | 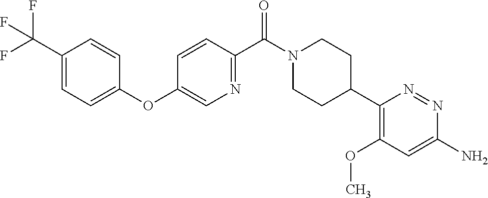 [SMILES: COc1cc(N)nnc1C1CCN(C(=O)c2ccc(Oc3ccc(C(F)(F)F)cc3)cn2)CC1] | 5-Methoxy-6-(1-{5-[4- (trifluoromethyl)-phenoxy]-pyridine-2- carbonyl}piperidin-4-yl)-pyridazin-3- amine |
| 92 | 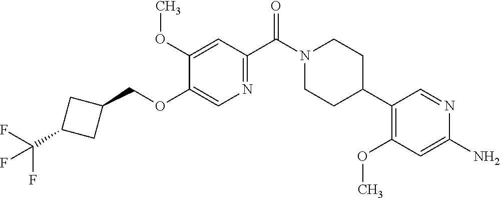 [SMILES: COc1cc(C(=O)N2CCC(c3cnc(N)cc3OC)CC2)ncc1OC[C@H]1C[C@H](C(F)(F)F)C1] | 4-Methoxy-5-[1-(4-methoxy-5-{[trans- 3-(trifluoromethyl)cyclobutyl]- methoxy}pyridine-2-carbonyl)- piperidin-4-yl]pyridin-2-amine |
| 93 | 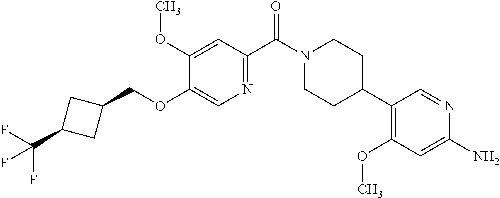 [SMILES: COc1cc(C(=O)N2CCC(c3cnc(N)cc3OC)CC2)ncc1OC[C@H]1C[C@@H](C(F)(F)F)C1] | 4-Methoxy-5-[1-(4-methoxy-5-{[(cis- 3-(trifluoromethyl)- cyclobutyl]methoxy}-pyridine-2- carbonyl)piperidin-4-yl]pyridin-2- amine |
| 94 | 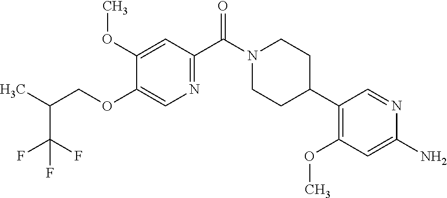 [SMILES: COc1cc(C(=O)N2CCC(c3cnc(N)cc3OC)CC2)ncc1OCC(C)C(F)(F)F] | 4-Methoxy-5-(1-{4-methoxy-5-[(2)- 3,3,3-trifluoro-2-methylpropoxy]- pyridine-2-carbonyl}piperidin-4- yl)pyridin-2-amine |
| 95 | 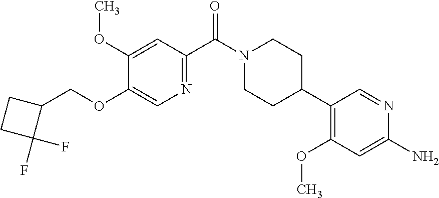 [SMILES: COc1cc(C(=O)N2CCC(c3cnc(N)cc3OC)CC2)ncc1OCC1CCC1(F)F] | 5-(1-{5-[(2,2-Difluorocyclobutyl)- methoxy]-4-methoxy-pyridine-2- carbonyl}-piperidin-4-yl)-4- methoxypyridin-2-amine |

In one embodiment, the invention relates to any of the methods described herein, wherein the compound of the invention is selected from any one of the compounds 1 to 95 depicted in Table 1 above, and the pharmaceutically acceptable salts thereof.

In another embodiment, the invention relates to any of the methods described herein, wherein the compound of the invention is selected from any one of the compounds 6, 16, 17, 33, 34, 40, 41, 44, 54, 57, 80, 83 and 88 depicted in Table 1; and the pharmaceutically acceptable salts thereof.

In another embodiment, the invention relates to any of the methods described herein, wherein the compound of the invention is selected from any one of the compounds 29, 31, 49, 56, 66, 85, 87, and 90 depicted in Table 1; and the pharmaceutically acceptable salts thereof.

In another embodiment, the invention relates to any of the methods described herein, wherein the compound of the invention is selected from the group consisting of:
[4-(6-Amino-pyridazin-3-yl)-piperidin-1-yl]-[5-(4-isopropoxy-phenoxy)-4-methoxy-pyridin-2-yl]-methanone;
[4-(6-Amino-pyridazin-3-yl)-piperidin-1-yl]-(4-methoxy-5-phenoxy-pyridin-2-yl)-methanone;
[4-(6-Amino-pyridazin-3-yl)-piperidin-1-yl]-[5-(4-fluoro-phenoxy)-4-methoxy-pyridin-2-yl]-methanone;
[4-(6-Amino-pyridazin-3-yl)-piperidin-1-yl]-[4-methoxy-5-(4-trifluoromethyl-phenoxy)-pyridin-2-yl]-methanone;
[4-(6-Amino-pyridazin-3-yl)-piperidin-1-yl]-[5-(4-chloro-phenoxy)-4-methoxy-pyridin-2-yl]-methanone;
[4-(6-Amino-pyridazin-3-yl)-piperidin-1-yl]-[5-(4-cyclopropoxy-phenoxy)-4-methoxy-pyridin-2-yl]-methanone;
[4-(6-Amino-pyridazin-3-yl)-piperidin-1-yl]-[5-(4-fluoro-benzyloxy)-4-methoxy-pyridin-2-yl]-methanone;
[4-(6-Amino-pyridazin-3-yl)-piperidin-1-yl]-[4-methoxy-5-(4-methoxy-phenoxy)-pyridin-2-yl]-methanone;
[4-(6-Amino-pyridazin-3-yl)-piperidin-1-yl]-[5-(4-difluoromethoxy-phenoxy)-4-methoxy-pyridin-2-yl]-methanone;
[4-(6-Amino-pyridazin-3-yl)-piperidin-1-yl]-[5-(2-fluoro-benzyloxy)-4-methoxy-pyridin-2-yl]-methanone;
[4-(6-Amino-pyridazin-3-yl)-piperidin-1-yl]-[4-methoxy-5-(4-trifluoromethoxy-phenoxy)-pyridin-2-yl]-methanone;
[4-(6-Amino-pyridazin-3-yl)-piperidin-1-yl]-[5-(phenoxy)-4-ethoxy-pyridin-2-yl]-methanone; and [4-(6-Amino-pyridazin-3-yl)-piperidin-1-yl]-[5-(4-fluoro-phenoxy)-4-ethoxy-pyridin-2-yl]-methanone;
   or a pharmaceutically acceptable salt thereof.

In another embodiment, the invention relates to any of the methods described herein, wherein the compound of the invention is selected from the group consisting of:
[4-(6-Amino-4-methyl-pyridazin-3-yl)-piperidin-1-yl]-[5-(4-fluoro-phenoxy)-4-methoxy-pyridin-2-yl]-methanone,
[4-(6-Amino-4-methyl-pyridazin-3-yl)-piperidin-1-yl]-(4-methoxy-5-phenoxy-pyridin-2-yl)-methanone,
[4-(6-Amino-4-methoxy-pyridazin-3-yl)-piperidin-1-yl]-[5-(4-fluoro-phenoxy)-4-methoxy-pyridin-2-yl]-methanone,
[4-(6-Amino-4-methoxy-pyridazin-3-yl)-piperidin-1-yl]-[4-methoxy-5-(4-trifluoromethyl-phenoxy)-pyridin-2-yl]-methanone,
[4-(6-Amino-4-methoxy-pyridazin-3-yl)-piperidin-1-yl]-[4-methoxy-5-(4-methoxy-phenoxy)-pyridin-2-yl]-methanone,
[4-(6-Amino-4-ethoxy-pyridazin-3-yl)-piperidin-1-yl]-[4-methoxy-5-(phenoxy)-pyridin-2-yl]-methanone,
5-Ethoxy-6-(1-{4-methoxy-5-[4-(trifluoromethyl)phenoxy]pyridine-2-carbonyl}piperidin-4-yl)pyridazin-3-amine, and
6-(1-{4-Methoxy-5-[4-(trifluoromethyl)phenoxy]pyridine-2-carbonyl}piperidin-4-yl)-5-methylpyridazin-3-amine,
   or a pharmaceutically acceptable salt thereof.

In another embodiment, the invention relates to any of the methods described herein, wherein the compound of the invention is [4-(6-Amino-pyridazin-3-yl)-piperidin-1-yl]-[5-(4-fluoro-phenoxy)-4-methoxy-pyridin-2-yl]-methanone (compound 17 depicted in Table 1); and the pharmaceutically acceptable salts thereof.

In another embodiment, the invention relates to any of the methods described herein, wherein the compound of the invention is 6-(1-{4-Methoxy-5-[4-(trifluoromethyl)phenoxy]pyridine-2-carbonyl}piperidin-4-yl)-5-methylpyridazin-3-amine (compound 90 depicted in Table 1); and the pharmaceutically acceptable salts thereof.

The terms “treatment” and “treating” comprise therapeutic treatment of patients having already developed one of the conditions described herein, in particular in manifest form. Therapeutic treatment may be symptomatic treatment in order to relieve the symptoms of the specific indication or causal treatment in order to reverse or partially reverse the conditions of the indication or to stop or slow down progression of the disease. Thus the compositions and methods of the present invention may be used for instance as therapeutic treatment over a period of time as well as for chronic therapy.

The TRPC6 inhibitors of the invention may be prepared according to the methods described in WO2019081637.

### General Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals, or moieties defined below, the number of carbon atoms is often specified preceding the group, for example, C₁₋₆-alkyl means an alkyl group or radical having 1 to 6 carbon atoms. In general, in groups like HO, H₂N, (O)S, (O)₂S, NC (cyano), HOOC, F₃C or the like, the skilled artisan can see the radical attachment point(s) to the molecule from the free valences of the group itself. For combined groups comprising two or more subgroups, the last named subgroup is the radical attachment point, for example, the substituent “aryl-C₁₋₃-alkyl” means an aryl group, which is bound to a C₁₋₃-alkyl-group, the latter of which is bound to the core or to the group to which the substituent is attached.

In case a compound is depicted in form of a chemical name and as a formula in case of any discrepancy the formula shall prevail.

The term “substituted” as used herein, means that any one or more hydrogens on the designated atom is replaced with a selection from the indicated group, provided that the designated atom's normal valence is not exceeded, and that the substitution results in a stable compound.

Unless specifically indicated, throughout the specification and the appended claims, a given chemical formula or name shall encompass tautomers and all stereo, optical and geometrical isomers (e.g. enantiomers, diastereomers, E/Z isomers etc.) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates of the free compounds or solvates of a salt of the compound.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, and commensurate with a reasonable benefit/risk ratio.

As used herein, “pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include acetates, ascorbates, benzenesulfonates, benzoates, besylates, bicarbonates, bitartrates, bromides/hydrobromides, edetates, camsylates, carbonates, chlorides/hydrochlorides, citrates, edisylates, ethane disulfonates, estolates esylates, formates, fumarates, gluceptates, gluconates, glutamates, glycolates, glycollylarsnilates, hexylresorcinates, hydrabamines, hydroxymaleates, hydroxynaphthoates, iodides, isothionates, lactates, lactobionates, malates, maleates, mandelates, methanesulfonates, methylbromides, methylnitrates, methylsulfates, mucates, napsylates, nitrates, oxalates, pamoates, pantothenates, phenylacetates, phosphates/diphosphates, polygalacturonates, propionates, salicylates, stearates, subacetates, succinates, sulfamides, sulfates, tannates, tartrates, teoclates, toluenesulfonates, triethiodides, trifluoroacetates, ammonium, benzathines, chloroprocaines, cholines, diethanolamines, ethylenediamines, meglumines and procaines. Further pharmaceutically acceptable salts can be formed with cations from metals like aluminum, calcium, lithium, magnesium, potassium, sodium, zinc and the like (also see Pharmaceutical salts, Birge, S. M. et al., J. Pharm. Sci., (1977), 66, 1-19) or with cations from ammonia, L-arginine, calcium, 2,2′-iminobisethanol, L-lysine, magnesium, N-methyl-D-glucamine, potassium, sodium and tris(hydroxymethyl)-aminomethane.

The term halogen generally denotes fluorine, chlorine, bromine and iodine.

The term “C₁₋ₙ alkyl”, wherein n is an integer selected from the group consisting of 2, 3, 4, 5 or 6, preferably 4 or 6, either alone or in combination with another radical denotes an acyclic, saturated, branched or linear hydrocarbon radical with 1 to n C atoms. For example the term C₁₋₅ alkyl embraces the radicals H₃C, H₃C CH₂, H₃C CH₂CH₂, H₃CCH(CH₃), H₃CCH₂CH₂CH₂, H₃CCH₂CH(CH₃), H₃CCH(CH₃)CH₂, H₃CC(CH₃)₂, H₃CCH₂CH₂CH₂CH₂, H₃CCH₂CH₂CH(CH₃), H₃CCH₂CH(CH₃)CH₂, H₃CCH(CH₃)CH₂CH₂, H₃CCH₂C(CH₃)₂, H₃CC(CH₃)₂CH₂, H₃CCH(CH₃)CH(CH₃) and H₃CCH₂CH(CH₂CH₃).

The term “C₃₋ₙ cycloalkyl”, wherein n is an integer from 4 to n, either alone or in combination with another radical denotes a cyclic, saturated, unbranched hydrocarbon radical with 3 to n C atoms. For example, the term C₃₋₇ cycloalkyl includes cyclopropyl, cyclobutyl, cyclopentyl, cyclohexyl and cycloheptyl.

By the term “halo” added to an “alkyl”, “alkylene” or “cycloalkyl” group (saturated or unsaturated) is such an alkyl or cycloalkyl group wherein one or more hydrogen atoms are replaced by a halogen atom selected from among fluorine, chlorine or bromine, preferably fluorine and chlorine, particularly preferred is fluorine. Examples include: H₂FC—, HF₂C—, F₃C—. Analogously, the term “halo” added to an aryl group (e.g., phenyl) means that one or more hydrogen atoms are replaced by a halogen atom selected from among fluorine, chlorine or bromine, preferably fluorine and chlorine, particularly preferred is fluorine.

The term “carbocyclyl” as used either alone or in combination with another radical, means a mono bi or tricyclic ring structure consisting of 3 to 9 carbon atoms and optionally a heteroatom selected from the group consisting of N, O, and S. The term “carbocyclyl” refers to fully saturated ring systems and encompasses fused, bridged and spirocyclic systems.

Many of the terms given above may be used repeatedly in the definition of a formula or group and in each case have one of the meanings given above, independently of one another.

Unless specifically indicated, throughout the specification and the appended claims, a given chemical formula or name shall encompass tautomers and all stereo, optical and geometrical isomers (e.g. enantiomers, diastereomers, E/Z isomers, etc.) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates of the free compounds or solvates of a salt of the compound.

Some of the compounds in Table 1 can exist in more than one tautomeric form. The invention includes methods for using all such tautomers.

In addition, within the scope of the invention is the use of prodrugs of the TRPC6 inhibitors within the methods of treatment of the invention. Prodrugs include those compounds that, upon simple chemical transformation, are modified to produce compounds of the invention.

Simple chemical transformations include hydrolysis, oxidation and reduction. Specifically, when a prodrug is administered to a patient, the prodrug may be transformed into a compound disclosed hereinabove, thereby imparting the desired pharmacological effect.

For all compounds disclosed herein above in this application, in the event the nomenclature is in conflict with the structure, it shall be understood that the compound is defined by the structure.

### Dosage Forms and Administration

Conventional dosage forms typically include a pharmaceutically acceptable carrier suitable to the particular dosage form selected. Routes of administration include, but are not limited to, intravenously, intramuscularly, subcutaneously, intrasynovially, by infusion, sublingually, transdermally, orally, topically or by inhalation. The preferred modes of administration are oral and intravenous.

The compounds of this invention may be administered alone or in combination with adjuvants that enhance stability of the inhibitors, facilitate administration of pharmaceutical compositions containing them in certain embodiments, provide increased dissolution or dispersion, increase inhibitory activity, provide adjunct therapy, and the like, including other active ingredients. In one embodiment, for example, multiple compounds of the present invention can be administered. Advantageously, such combination therapies utilize lower dosages of the conventional therapeutics, thus avoiding possible toxicity and adverse side effects incurred when those agents are used as monotherapies. Compounds of the invention may be physically combined with the conventional therapeutics or other adjuvants into a single pharmaceutical composition. Advantageously, the compounds may then be administered together in a single dosage form. In some embodiments, the pharmaceutical compositions comprising such combinations of compounds contain at least about 5%, but more preferably at least about 20%, of a compound of the invention (w/w) or a combination thereof. The optimum percentage (w/w) of a compound of the invention may vary and is within the purview of those skilled in the art. Alternatively, the compounds of the present invention and the conventional therapeutics or other adjuvants may be administered separately (either serially or in parallel). Separate dosing allows for greater flexibility in the dosing regimen.

As mentioned above, dosage forms of the compounds of this invention may include pharmaceutically acceptable carriers and adjuvants known to those of ordinary skill in the art and suitable to the dosage form. These carriers and adjuvants include, for example, ion exchangers, alumina, aluminum stearate, lecithin, serum proteins, buffer substances, water, salts or electrolytes and cellulose-based substances. Preferred dosage forms include tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements for the compounds of the present invention may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

The compounds of the invention may be used alone or in combination of one or more additional therapeutic agents. Nonlimiting examples of additional therapeutic agents may include:
Adrenocorticotropic hormone (ACTH);
Aldosterone inhibitors such as those described in WO 2016/014736;
Angiotensin-converting enzyme (ACE) inhibitors such as cilazapril, enalapril, captopril, benazepril, or lisinopril;
Antihypertensives such as reserpine, hydralazine or prazosin;
Angiotensin II receptor blocker (ARBs: such as candastartan, irbesartan, or losartan;
Calcineurin inhibitors (CNIs) such as cyclosporine tacrolimus;
Calcium channel blockers or inhibitors such as isradipine;
CD20 monoclonal antibodies such as rituximab;
Corticosteroid therapies such as prednisone or dexamethasone (including high-dose dexamethasone);
Cytotoxic agents such as cyclophosphamide or chlorambucil;
Diuretics such as furosemide;
Immunosupressants such as mycophenolate mofetil (MMF), prednisolone, methylprednisolone, corticosteroids, cyclosporine, cyclosporine A, or adalimumab;
Mineralocorticoid receptor antagonist such as finerenone;
Renin angiotensin aldosterone system (RAAS) mediators/inhibitors; and
SGLT2i such as empagliflozin, dapagliflozin, or canagliflozin;

### Description of Design and Trial Population

The use of the compounds of the invention to treat FSGS may be shown in the below-described clinical trial.

Applicant found a higher proportion of patients treated with TRPC6 inhibition with Compound 17 show a proteinuria reduction of more than 25% vs their baseline level as compared to patients treated with placebo.

### 1.1 Overall Trial Design

This multicenter, randomized, double-blind, parallel group study will assess 3 doses of a TRPC6 inhibitor of the invention compared to placebo administered orally once daily for 12 weeks in patients with primary or TRPC6 monogenic FSGS.

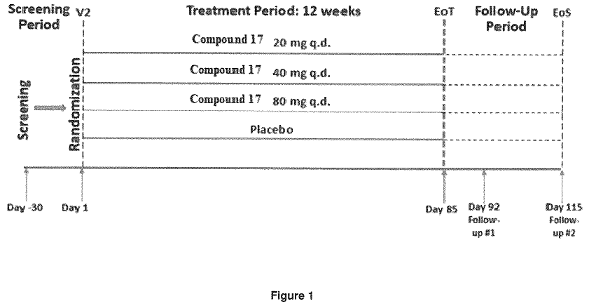

After completion of all the screening procedures, eligible patients will be randomized into one of the 4 treatment groups stratified by use of corticosteroids and treated for 12 weeks. It is planned to randomize approximately 15 patients in each treatment group. At the end of the treatment period, there will be a follow-up visit at day 7 after end of treatment. A 2^{nd} follow-up visit will be scheduled via telephone, 30 days from end of treatment. Eye exams will be completed at the 2^{nd} follow-up visit. A schematic illustration of the trial design is presented in FIG. 1.

### 1.2 Discussion of Trial Design, Including the Choice of Control Group

A randomized, double-blind, placebo-controlled design is selected for this study. It is standard in studies at this early stage of development to use placebo as a control group in order to evaluate efficacy, safety and tolerability. The conduct of this short duration proof of clinical principal study will be on top of an array of established standard of care modalities including conservative management and steroid-based immunosuppression regimens.

Most FSGS studies on glomerular diseases have used different metrics of proteinuria including UPCR. UPCR will be measured from 24-hour urine in this study.

UPCR reduction ratio (follow-up vs. baseline) for Cyclosporine A (CSA) and mycophenolate mofetil/dexamethasone (MMF/DEX) in a study with adults and children with steroid resistant FSGS was analyzed. Gipson D S, et al., “Clinical trial of focal segmental glomerulosclerosis in children and young adults,” Kidney Int 2011; 80(8):868-878. At week 8, there were approximately 65% and 50% reduction of UPCR for CSA and MMF/DEX, respectively. A 12-week treatment to identify a reduction in UPCR should therefore be sufficient to demonstrate efficacy in the study. In addition, efficacy will further be assessed in this study by the evaluation of UACR and 24-hour proteinuria as secondary endpoints.

### 1.3 Selection of Trial Population

This study will randomize approximately 60 patients with primary FSGS or with TRPC6 mutations causing FSGS. It is planned to conduct the study in approximately 55 sites in multiple countries. A sufficient number of patients will be screened to meet the randomization goal.

Study sites in selected countries will have the option of integrating a decentralized clinical trial (DCT) model where the study visits are conducted outside of a dedicated healthcare or research facility. This would allow patients to participate in the trial remotely and complete the study in their own home or residence. The DCT model can bring the clinical trial to patient's homes through telemedicine, a smartphone device, and through the deployment of mobile research nurses (MRN). The MRN will visit the patient's home and complete trial procedures in collaboration with the site principal investigator and study staff.

Sites participating in the DCT model would also continue to enroll patients using the traditional site-based approach where the patient visits the clinic at the specified time points in the protocol. Sites with DCT integration can also allow active ongoing patients in the trial to switch to remote participation. Additional operational guidelines will be provided in a separate DCT operational manual. A copy of the operational manual will be provided in the ISF.

Recruitment of patients for this trial is competitive, i.e. screening for the trial will stop at all sites at the same time once a sufficient number of patients has been screened. Investigators will be notified about screening completion and will then not be allowed to screen additional patients for this trial. Patients already in screening at this time may be allowed to continue to randomization if eligible.

A log of all patients enrolled into the trial (i.e. who have signed informed consent including patients who signed a screening consent or provided verbal consent) will be maintained in the ISF irrespective of whether they have been treated with investigational drug or not. If retrospectively it is found that a patient has been randomized in error (did not meet all inclusion criteria or met one or more exclusion criteria), the sponsor or delegate should be contacted immediately. Based on individual benefit-risk assessment, a decision will be made whether continued trial participation is possible.

### 1.3.1 Main Diagnosis for Trial Entry

The study will include patients with primary FSGS, and patients with monogenic FSGS as a result of TRPC6 mutations.

### 1.3.2 Inclusion Criteria

1. Signed and dated informed consent in accordance with ICH-GCP and local legislation prior to admission to the study.
2. Male and female patients 18 years to 75 years (both inclusive) of age on the day of signing informed consent.
3. Patients diagnosed with biopsy proven primary FSGS or documented TRPC6 gene mutation causing FSGS prior to screening visit.
4. UPCR≥1000 mg/g based on first morning void urine sample during screening.
5. Patients treated with corticosteroids must be on a stable dose for at least 4 weeks prior to screening visit with no plan to change the dose until end of trial treatment.
6. Patients treated with ACE inhibitors, ARBs, finerenone, aldosterone inhibitors, or SGLT2 inhibitors should be on a stable dose for at least 4 weeks prior to screening visit with no plan to change the dose until end of trial treatment.
7. Body Mass Index (BMI) of ≤40 kg/m² at screening visit.
8. Women of childbearing potential (WOCBP¹) must be willing and able to use highly effective methods of birth control per ICH M3 (R²) that result in a low failure rate of less than 1% per year when used consistently and correctly. A list of contraception methods meeting these criteria is provided in the informed consent form (ICF) and as described herein.

### 1.3.3 Exclusion Criteria

1. Known monogenic (with the exception of TRPC6 gene mutations) or clinical or histologic evidence of secondary FSGS.
2. Documented Alport syndrome, Nail Patella syndrome, diabetic nephropathy, IgA-nephropathy, lupus nephritis, or monoclonal gammopathy (e.g., multiple myeloma).
3. Genito-urinary malformations with vesicoureteral reflux or renal dysplasia.
4. A history of organ transplantation or planned transplantation during the course of the study.
5. Uncontrolled hypertension defined as an average resting systolic blood pressure>160 mmHg calculated from the last two of the triplicates sitting blood pressure measurements at screening visit. Patients with a documented history of white coat hypertension may be included.
6. Concomitant use of calcineurin inhibitors within 5 half-lives before screening visit.
7. Concomitant treatment with cytotoxic agents (cyclophosphamide, chlorambucil), or CD20 monoclonal antibody, e.g., rituximab, within 5 half-lives before screening visit. Note: use of other immunosuppression therapies considered as standard of care may be allowed as long as the patient remains on stable dose throughout the study.
8. Treatment with metformin or dofetilide (MATE1 or OCT2 substrates); dabigatran or digoxin (P-gp substrates with narrow therapeutic window) within 5 half-lives before screening visit.
9. Treatment with strong inhibitors or strong inducers of CYP3A within 1 week or 5 half-lives before screening visit (whichever is longer).
10. Estimated glomerular filtration rate (eGFR)<30 mL/min/1.73 m² (CKD-EPI formula based on serum creatinine and cystatin C) at screening visit.
11. Alanine aminotransferase (ALT)/aspartate aminotransferase (AST)>3× the upper limit of normal (ULN) at screening visit.
12. Clinically significant laboratory abnormalities or medical conditions which pose a safety risk for the patient or may interfere with the trial objectives in the investigator's opinion (except for renal function tests or deviation of clinical laboratory values that are related to FSGS) at screening visit.
13. QTc intervals (QTcF) greater than 450 ms in males or greater than 470 ms in females, or any other clinically relevant ECG findings (at the investigator's discretion) at screening visit.
14. History of congenital long QT syndrome, previous drug-induced QT prolongation, or other risk factors for Torsade de pointes (e.g. hypokalemia, bradycardia, heart failure).
15. Detection of graded cataract by LOCS Ill higher than NC1/NO1, C0, P0 in the slit lamp eye examination at screening visit. Planned cataract surgery during participation in the study. Patients with cataract who have undergone lens replacement are not excluded.
16. A history of gastrointestinal (GI) surgery or GI disorders that could interfere with absorption of trial medication in the investigator's opinion.
17. Major surgery (major according to the investigator's assessment, e.g. hip replacement) performed 3 months before the screening visit or planned within 6 months after entering the study.
18. Any documented active or suspected malignancy or history of malignancy within 5 years prior to screening visit, except appropriately treated basal cell carcinoma of the skin or in situ carcinoma of uterine cervix.
19. History of relevant allergy or hypersensitivity according to the investigator's clinical judgment (e.g. systemic hypersensitivity reactions including anaphylaxis and anaphylactoid reactions to the excipients or any other systemically administered agent).
20. Patients who need to use restricted medications (see Other Treatments, Emergency Procedures, Restrictions) or any drug considered likely to interfere with the safe conduct of the study, e.g. drugs with known QT-prolongation effects.
21. Patients not expected to comply with the protocol requirements or not expected to complete the study as scheduled (e.g. chronic alcohol or drug abuse or any other condition that, in the investigator's opinion, makes the patient an unreliable study participant).
22. Previous enrollment in this trial, or currently enrolled in another study investigating a device or drug, or less than 30 days or 5 times half-life of the investigational drug (whichever is longer) since ending another investigational drug study. Patients participating in observational studies will not be excluded.
23. Women who are pregnant, nursing, or who plan to become pregnant while in the study.
24. A positive test for SARS-CoV-2 during the screening period and up to the randomization.
   1.3.4 Discontinuation of Patients from Treatment or Assessments

Patients may discontinue trial treatment or withdraw consent to trial participation as a whole (“withdrawal of consent”) with very different implications (see Discontinuation of Patients from Treatment or Assessments). Every effort should be made to keep the patients in the trial. Measures to control the withdrawal rate include careful patient selection, appropriate explanation of the trial requirements and procedures prior to trial enrollment, as well as the explanation of the consequences of withdrawal. The decision to discontinue trial treatment or withdraw consent to trial participation and the reason must be documented in the patient files and electronic case report forms (eCRF). If applicable, consider the requirements for AE collection reporting (section Assessment of Adverse Events below).

### Discontinuation of Trial Treatment

An individual patient will discontinue trial treatment if:
The patient wants to discontinue trial treatment. The patient will be asked to explain the reasons but has the right to refuse to answer.
The patient has repeatedly shown to be non-compliant with important trial procedures and, in the opinion of both, the investigator and sponsor representative, the safety of the patient cannot be guaranteed as he/she is not willing or able to adhere to the trial requirements in the future.
The patient needs to take concomitant medication which may increase their risk of adverse effects or interfere with the investigational medicinal product (see Other Treatments, Emergency Procedures, Restrictions below). In cases where treatment is expected to be temporary (short course), the case should be discussed with the sponsor and a decision will be made on potentially restarting trial medication.
The patient can no longer receive trial treatment for medical reasons (such as surgery, serious or severe drug induced liver injury attributable to the trial drug (see Assessment of Adverse Events below), other AEs, other diseases, or pregnancy).
The patient experiences confirmed QT prolongation of QTcF intervals greater than 500 ms or an increase of >60 ms compared to baseline (defined as visit 2, pre-dose measurement).
The patient experiences clinically relevant conduction disorders (e.g. AV block≥2^{nd} degree, bundle branch block).
The patient is diagnosed with cataract (graded by LOCS Ill higher than NC1/NO1, C0, P0) during study treatment.
eGFR below 25 mL/min/1.73 m² (CKD-EPI formula based on serum cystatin C) and not improved by resolution of transient events (e.g. dehydration).

Patient experiences acute kidney injury as per clinical judgement by the investigator and/or according to the modified Kidney Disease: Improving Global Outcomes (KDIGO) definition. See “Kidney Disease: Improving Global Outcomes (KDIGO) Acute Kidney Injury Work Group. KIDGO clinical practice guideline for acute kidney injury,” Kidney Int Suppl 2012; 2(1):1-138 and adapted for cystatin C as opposed to serum creatinine. Inker L A et al., “CKD-EPI Investigators. Estimating glomerular filtration rate from serum creatinine and cystatin C,” N Engl J Med 2012; 367(1):20-29 and Levey A S et al., “Glomerular filtration rate and albuminuria for detection and staging of acute and chronic kidney disease in adults: a systematic review,” JAMA 2015; 313(8):837-846.
Increase in cystatin C to 1.5 times the baseline value, which is known or presumed to have occurred within the prior 7 days.
The patient is diagnosed with severe COVID-19 (for example patients who require hospitalization, need supplemental oxygen, have compromised lung function, etc.). If the patient has mild illness, continuation of trial treatment is at the discretion of the investigator. Testing for SARS-CoV-2 infection must be done locally and not at the central lab. Positive test should be reported as an AE, and reason for premature withdrawal should be recorded as “other AE” if applicable.

If new efficacy/safety information becomes available, the benefit-risk-assessment will be reviewed and, if needed, paused or discontinued the trial treatment for all patients or take any other appropriate action to guarantee the safety of the trial patients.

If the trial treatment is permanently discontinued, patient should complete the EoT, follow-up (FUP1), and EoS visits as outlined in study procedures in Table 2.

TABLE 2
Study Procedures

| Trial Periods | Screening | Treatment Period | | | | | | Follow-up |
|---|---|---|---|---|---|---|---|---|
| Visit¹ | 1 | 2 | 3 | 4 | 5 | EoT² | FUP1 | EoS³ |
| Week | | 0 | | 4 | 8 | 12 | 13 | |
| Days from first dose | −30⁴ | 1⁵ | 4 | 29 | 57 | 85 | 92 | 115 |
| Time window for visits | | 0 | ±3 | ±3 | ±3 | ±3 | ±2 | ±3 |
| (days) | | | | | | | | |
| Informed consent⁶ | X | | | | | | | |
| Demographics | X | | | | | | | |
| Height⁷, body weight | X | | | X | X | X | X | |
| Medical history | X | X | | | | | | |
| Review of inclusion/ | X | X | | | | | | |
| exclusion criteria | | | | | | | | |
| Physical exam⁸ | X | | | X | | X | | |
| Vital signs²⁸ | X | X | X | X | X | X | X | |
| 12-lead ECG⁹ | X | X | X | X | X | X | X | |
| Sampling for safety | X | X | X | X | X | X | X | |
| laboratory (incl. serum | | | | | | | | |
| creatinine/cystatin C for | | | | | | | | |
| eGFR) | | | | | | | | |
| Pregnancy testing¹⁰ | X | X | | X | X | X | X | |
| Eye exams¹¹ | X | | | | | X¹² | | X¹² |
| First morning void | X | | | | | | | |
| (FMV) | | | | | | | | |
| Dispense 24-hr urine | X | X | | | X | X | | |
| collection containers¹³ | | | | | | | | |
| 24-hour urine collection | XX¹⁴ | | X¹⁵ | | | X¹⁶ | X¹⁷ | |
| Spot Urine¹⁸ | | X | X | X | X | X | X | |
| Biomarker sampling | | X | | X | X | X | X | |
| (blood, urine)¹⁹ | | | | | | | | |
| Pharmacogenomics²⁰ | | X | | | | | | |
| PK sampling (blood)²¹ | | X | X | X | X | X | X²² | |
| Randomization & | | X | | | | | | |
| dispense trial | | | | | | | | |
| medication²³ | | | | | | | | |
| Dispense/review of | | X | X | X | X | X | | |
| medication diary²⁴ | | | | | | | | |
| Medication | | X | X | X | X | X | | |
| administration during | | | | | | | | |
| study visit²⁵ | | | | | | | | |
| Concomitant therapy | X | X | X | X | X | X | X | X |
| All AEs²⁶/SAEs/AESIs | X | X | X | X | X | X | X | X |
| Return medication/ | | | X | X | X | X | | |
| compliance check²⁷ | | | | | | | | |
| Completion of patient | | | | | | | | X |
| participation | | | | | | | | |

### Footnotes

1. Study visits may be completed at the patient's home for patients participating in the trial remotely via the decentralized clinical trial (DCT) model. All trial procedures performed at the clinic can be completed by the mobile research nurse (MRN) at the patient's home. For eye examinations (see footnotes 11 and 12).
2. End of Treatment (EoT) visit. Patients who discontinue trial treatment prematurely should undergo the End of Treatment (EoT) visit procedures as soon as possible.
3. End of Study (EoS) is synonym for End of Trial. EoS will be a telephone visit. For eye exams at EoS, see footnote 12. Patients who discontinue trial treatment prematurely should complete follow-up visit (FUP1) and EoS visit. Study site staff will complete the EoS visit by telephone for all patients.
4. Screening period can be shorter than 30 days. Screening period may also be extended if needed.
5. Day of first intake of trial medication. Randomization is completed prior to visit 2 and trial medication is shipped directly to the patient.
6. To allow collection of the first morning void sample to determine if the patient's UPCR meets the protocol inclusion criteria prior to visit 1, patients may sign a separate screening consent form (or verbally consent). Patients who are eligible per their UPCR results must sign the main consent form prior to additional screening procedures being performed. Once the patient has consented to collect the first morning void urine sample, the patient is considered to be enrolled in the trial and must be registered in the IRT.
7. Height measurement collected only at the screening visit.
8. Physical exam for patients participating in the trial remotely will be performed via telemedicine.
9. ECGs will be recorded at the screening visit. Starting first dosing visit (visit 2) to EoT visit, ECGs will be recorded twice: pre-dose and at 1 to 2 hours post-dose. At the FUP1 visit, there will be one ECG recording. See additional guidelines on ECG recordings in section.
   a. ECGs at the screening visit will be recorded in triplicate (3 single ECGs recorded within 180 seconds). The average of the 3 readings will be used to determine eligibility.
   b. ECGs at the screening visit will be recorded in triplicate (3 single ECGs recorded within 180 seconds). The average of the 3 readings will be used to determine eligibility.
   c. For patients participating in the trial remotely, the MRN will complete the ECG procedure at the patient's home.
10. Serum pregnancy test at screening visit (test performed at central lab), and urine pregnancy test at the study site for other visits (except visit 3 and phone visits). Menstrual cycle status (not delayed or missed period) should be checked before the first dose (visit 2). Applicable to only women of childbearing potential.
11. Eye exams should be completed at the study site or at another healthcare or eye care facility. If documented medical history of cataract surgery in both eyes is available at the time of screening, eye assessments are not required in this trial.
12. If cataract surgery in both eyes is confirmed by the eye exam at screening, patients are not required to complete the eye exams at EoT and EoS.
13. Patients will receive instructions on urine collection, storage, and return of the specimens to the study site. If possible, patients should be reminded (e.g., via telephone) during screening and ahead of the applicable visits to collect the 24-hour urine samples.
14. Two separate 24-hour urine samples will be collected on separate days prior to visit 2. The 24-hour urine samples should be collected only after confirmation that the patient met all the eligibility criteria for the trial, and the collections should be close to visit 2 as much as possible. Visit 2 must be rescheduled if UPCR data is not available at least from one 24-hour urine sample.
15. One 24-hour urine sample should be collected before visit 3.
16. One 24-hour urine samples should be collected before EoT visit. EoT visit must be rescheduled if the 24-hour urine sample is not collected for UPCR measurements.
17. One 24-hour urine sample will be collected before FUP1. For information and collection time points, see section 6.1.
18. Spot urine will be collected for UACR and UPCR calculations. Urine for this purpose cannot be taken from the 24-hour urine sample.
19. Spot urine samples for biomarkers will be collected during the study visit. Blood samples for serum and plasma biomarkers will be collected at the same time as the first PK sample is drawn. Urine for this purpose cannot be taken from the 24-hour urine sample. If the visits are conducted remotely, urine biomarker samples will not be collected.
20. One blood sample for pharmacogenomics will be taken at visit 2. If the sample is not collected at visit 2, it can be collected at a later visit.
21. Refer to Table 6 for planned PK sampling schedule. The date and exact time of trial medication administration will be recorded in the eCRF together with the date and exact time when PK samples are drawn.
22. An additional PK sample will be collected at the FUP1 visit.
23. Randomization will be performed using the IRT Platform at least a week before the scheduled visit 2 date. Randomization call must occur only after patient eligibility (including UPCR criteria, eye exams) has been confirmed and UPCR data from central lab is available from at least one 24-hour urine sample during screening period. Trial medication will be shipped directly to the patient via a courier from a central depot unless not permitted by local/site regulations. First dose will be administered at visit 2. A telephone call (video call if possible) from site to patient should occur in about 24 hours (or next working day) after the site receives the delivery notification from the courier.
24. A medication diary (paper) will be dispensed to the patient. Patient should record the date and time of medication intake at home for the 3 days prior to EoT, and for the previous day for all other clinic visits. Patients should bring the diary to the clinic for review. For more information, see section 4.1.4. If possible, a few days before the visit patients should be reminded (e.g., via telephone) to complete the medication diary, and to not take the study drug on the day of the study visit.
25. Patient will bring all the trial medication received directly from the central depot to the clinic for visit 2 (first dosing visit). Site will inspect all the trial medication and provide instruction to the patient on how medication is administered at home and how the medication should be brought to the clinic visits. Site will open the first bottle to administer the first dose in the clinic. When the patient is contacted after the trial medication is delivered to the patient's home (see footnote 23), they should be reminded to bring the trial medication to the clinic for visit 2. If possible, patients should be reminded (e.g. by telephone) to bring the trial medication to the clinic before each visit. Study visits will have to be rescheduled if trial medication is not available during the applicable visits. For information on how trial medication will be handled during the course of the trial, see section 4.1.4.
26. A separate eCRF will be used to collect specific data related to acute kidney injury. After the FUP1 visit until the individual patient's end of trial only cancers of new histology and exacerbations of existing cancer, all trial drug related SAEs and all trial drug related AESIs will be collected.
27. Medication compliance will be checked by capsule count by study site staff. For information on medication return.
28. Assessment for uncontrolled hypertension is completed at Visit 1 only. Three blood pressure measurements will be taken approximately 2 minutes apart after the patient has rested quietly and is in a seated position for at least 5 minutes. The average of the last two measurements for the triplicate systolic blood pressure measurements should be <160 mmHg for assessment of exclusion criteria 5 at the screening visit.

### Withdrawal of Consent to Trial Participation

Patients may withdraw their consent to trial participation at any time without the need to justify the decision.

If a patient wants to withdraw consent, the investigator should be involved in the discussion with the patient and explain the difference between trial treatment discontinuation and withdrawal of consent to trial participation. Investigator should also explain the options for continued follow-up after trial treatment discontinuation.

### Discontinuation of the Trial by the Sponsor

BI reserves the right to discontinue the trial overall or at a particular trial site at any time for the following reasons:
1. Failure to meet expected enrollment goals overall or at a particular trial site.
2. New efficacy or safety information invalidating the earlier positive benefit-risk-assessment.
3. Deviations from Good Clinical Practice (GCP), the trial protocol, or the contract impairing the appropriate conduct of the trial.

The investigator/the trial site will be reimbursed for reasonable expenses incurred in case of trial termination (except in case of the third reason).

### 2. Treatments

### 2.1 Investigational Treatments

### 2.1.1 Identity of the Investigational Medicinal Products

The general description of the investigational test products is shown in Table 3 (TRPCi medicinal product) and Table 4 (placebo).

TABLE 3
Medicinal test TRPC1 test product Compound 17 (product 1).

| | |
|---|---|
| Substance: | TRPC6 inhibitor of the invention |
| Pharmaceutical | Capsule |
| formulation: | |
| Unit strength: | Low dose (20 mg), moderate dose (40 mg) and high |
| | dose (80 mg) of the TRPC6 inhibitor |
| Posology: | q.d. |
| Mode of | Oral (p.o.) |
| administration: | |

TABLE 4
Matching placebo test product 2.

| | |
|---|---|
| Substance: | Placebo to match TRPC6 |
| | inhibitor of the invention |
| Pharmaceutical | Capsule |
| formulation: | |
| Unit strength: | Not applicable |
| Posology: | q.d. |
| Mode of | Oral (p.o.) |
| administration: | |

### 2.1.2 Selection of Doses in the Trial

The proposed dosage of the TRPC6 inhibitor is from 1 to 100 mg.

In one embodiment, the TRPC inhibitor is administered to the patient in an amount of 10 mg, or 12.5 mg, or 15 mg, or 17.5 mg, or 20 mg, or 22.5 mg, or 25 mg, or 27.5 mg, or 30 mg, or 32.5 mg, or 35 mg, or 37.5 mg, or 40 mg, or 42.5 mg, or 45 mg, or 47.5 mg, or 50 mg, or 52.5 mg, or 55 mg, or 57.5 mg, or 60 mg, or 62.5 mg, or 65 mg, or 67.5 mg, or 70 mg, or 72.5 mg, or 75 mg, or 77.5 mg, or 80 mg, or 82.5 mg, or 85 mg, or 87.5 mg, or 90 mg.

In another embodiment, the TRPC inhibitor is administered to the patient in an amount of 20 mg, 40 mg, or 80 mg.

In some embodiments, the proposed dosage may include a low dosage, moderate dosage, and/or a high dosage treatment regimen.

In one embodiment, the amount of the TRPC6 inhibitor in the low dose is from 1 to 30 mg. In another embodiment, the amount of the TRPC6 inhibitor in the low dose is from 5 to 30 mg. In another embodiment, the amount of the TRPC6 inhibitor in the low dose is from 10 to 25 mg. In another embodiment, the amount of the TRPC6 inhibitor in the low dose is from 15 to 25 mg.

In another embodiment, the amount of the TRPC6 inhibitor in the low dose is 10 mg, or 12.5 mg, or 15 mg, or 17.5 mg, or 20 mg, or 22.5 mg, or 25 mg, or 27.5 mg, or 30 mg.

In another embodiment, the amount of the TRPC6 inhibitor in the moderate dose is from 32.5 to 60 mg. In another embodiment, the amount of the TRPC6 inhibitor in the moderate dose is from 35 to 45 mg.

In another embodiment, the amount of the TRPC6 inhibitor in the moderate dose is 32.5 mg, or 35 mg, or 37.5 mg, or 40 mg, or 42.5 mg, or 45 mg, or 47.5 mg, or 50 mg, or 52.5 mg, or 55 mg, or 57.5 mg, or 60 mg.

In another embodiment, the amount of the TRPC6 inhibitor in the high dose is from 62.5 to 100 mg. In another embodiment, the amount of the TRPC6 inhibitor in the high dose is from 70 to 90 mg. In another embodiment, the amount of the TRPC6 inhibitor in the high dose is from 75 to 85 mg.

In another embodiment, the amount of the TRPC6 inhibitor in the high dose is 62.5 mg, or 65 mg, or 67.5 mg, or 70 mg, or 72.5 mg, or 75 mg, or 77.5 mg, or 80 mg, or 82.5 mg, or 85 mg, or 87.5 mg, or 90 mg, or 92.5 mg, or 95 mg, or 97.5 mg, or 100 mg.

In another embodiment, the amount of the TRPC6 inhibitor in the moderate dose is 1.25 times the amount in the low dose, and the amount of the TRPC6 inhibitor in the high dose is 2.5 times the amount in the low dose. In another embodiment, the amount of the TRPC6 inhibitor in the moderate dose is 1.5 times the amount in the low dose, and the amount of the TRPC6 inhibitor in the high dose is 3 times the amount in the low dose. In another embodiment, the amount of the TRPC6 inhibitor in the moderate dose is 2 times the amount in the low dose, and the amount of the TRPC6 inhibitor in the high dose is 4 times the amount in the low dose. In another embodiment, the amount of the TRPC6 inhibitor in the moderate dose is 2.25 times the amount in the low dose, and the amount of the TRPC6 inhibitor in the high dose is 4.5 times the amount in the low dose. In another embodiment, the amount of the TRPC6 inhibitor in the moderate dose is 2.5 times the amount in the low dose, and the amount of the TRPC6 inhibitor in the high dose is 5 times the amount in the low dose.

In another embodiment, the low dose, moderate dose, and high dose are 10 mg, 20 mg, and 30 mg, respectively. In another embodiment, the low dose, moderate dose, and high dose are 15 mg, 30 mg, and 45 mg, respectively. In another embodiment, the low dose, moderate dose, and high dose are 20 mg, 40 mg, and 60 mg, respectively. In another embodiment, the low dose, moderate dose, and high dose are 10 mg, 20 mg, and 40 mg, respectively. In another embodiment, the low dose, moderate dose, and high dose are 20 mg, 40 mg, and 80 mg, respectively. In another embodiment, the low dose, moderate dose, and high dose are 10 mg, 25 mg, and 50 mg, respectively. In another embodiment, the low dose, moderate dose, and high dose are 20 mg, 50 mg, and 100 mg, respectively.

In one embodiment, the TRPC6 inhibitor is administered to the patient once daily. In another embodiment, TRPC6 inhibitor is administered to the patient twice daily. In another embodiment, TRPC6 inhibitor is administered to the patient thrice daily.

In another embodiment, the TRPC6 inhibitor is administered to the patient in a total daily amount of 20 mg. In another embodiment, the TRPC6 inhibitor is administered to the patient in a total daily amount of 40 mg. In another embodiment, the TRPC6 inhibitor is administered to the patient in a total daily amount of 80 mg.

In one embodiment, the therapeutic dose of the TRPC6 inhibitor of the invention is between 20 and 40 mg. However, in the absence of the possibility to assess target engagement in humans and given the uncertainty of a demonstrable pharmacodynamic effect solely based on in vitro dose estimation, a higher exposure (at multiples of IC₉₀) may be needed to observe a therapeutic response in vivo.

From an efficacy perspective, in this mechanistic study, high doses of TRPC6 inhibitor of the invention (above the anticipated therapeutic dose) are investigated in order to explore whether TRPC6 inhibition induces a clinically meaningful UPCR response in patients with primary or TRPC6 monogenic FSGS. Beyond the establishment of the proof of clinical principle, which is a key objective in this trial, future studies, including a dedicated Phase 2 dose finding trial will enable a thorough investigation of therapeutic doses and the establishment of the minimum effective dose.

### 2.1.3 Method of Assigning Patients to Treatment Groups

After the assessment of all inclusion and exclusion criteria, each eligible patient will be randomized to one of the treatment groups according to the randomization plan in a 1:1:1:1 ratio via Interactive Response Technology (IRT). Randomization will be stratified by use of corticosteroids.

Note that the trial medication number is different from the patient number (the latter is generated during screening via the IRT System). A total of 60 patients will be randomized with approximately 15 patients in each treatment group. Patients randomized by error and did not receive any trial medication may be replaced.

### 2.1.4 Drug Assignment and Administration of Doses for Each Patient

Table 5 shows the trial medication schedule for each treatment group.

TABLE 5
TRPC6 inhibitor of the invention and placebo treatments

| | | | | Number of | Total |
|---|---|---|---|---|---|
| Dose | | Pharmaceutical | Unit | capsules per | daily |
| group | Substance | form | strength | administration | dose |
| 1 | TRPC6 | Capsule | 20 mg | 1 capsule q.d. | 20 mg |
| | inhibitor | | | for 12 weeks | |
| 2 | TRPC6 | Capsule | 40 mg | 1 capsule q.d. | 40 mg |
| | inhibitor | | | for 12 weeks | |
| 3 | TRPC6 | Capsule | 80 mg | 1 capsule q.d. | 80 mg |
| | inhibitor | | | for 12 weeks | |
| 1-3 | Placebo* | Capsule | — | Matching placebo | — |
| | | | | for 12 weeks | |

*Subjects receiving placebo are equally distributed across all three treatment groups.

After eligibility of the patient is confirmed during the screening period, patient will be randomized in the IRT Platform at least 7 days before the scheduled visit 2 date. Randomization will initiate the direct shipment of trial medication to the patient. Each patient will receive trial medication from an external depot contracted by the sponsor. Patient's name and address will not be made available to the sponsor. Patient will receive sufficient trial medication for the entire 12-week duration of the treatment period.

Prior to shipment of the trial medication, study site staff will train the patient on handling, storage, and use of the trial medication. Site staff should contact the patient by telephone (video call if possible) in about 24 hours (or next working day) after the site receives the delivery notification from the courier. During this contact, the site should remind the patient of the proper storage requirements for the medication. The patient should also be instructed not to open the trial medication package and to bring all the medication to the clinic for visit 2.

Medication from one of the bottles will be used to dose the patient at visit 2. Site staff will inspect the shipment to ensure patient has received all the medication. All trial medication (except the capsule used for dosing at visit 2) will be returned to the patient with instructions for administration of medication at home.
Patient will self-administer the trial medication at home. On study visit days, patient must receive the dose during the study visit (at the clinic or at home) after the pre-dose PK sample is taken.
Patients should take one capsule a day with or without food, preferably at the same time of the day in the mornings.
In case dosing occurs before 18:00 hours (6 p.m.), the next dose should be in the morning the next day (followed by morning dosing thereafter).
In case dosing occurs before midnight, the next dose should be at about noon the next day (followed by morning dosing thereafter).
If dosing does not occur on a particular day (not taken until midnight), the next dose should be in the morning the next day (followed by morning dosing thereafter).

The route of administration is by mouth (p.o.). Trial treatment may be restarted after temporary discontinuation. Dose reduction or increase are not allowed.

On study visit days, trial medication will be administered at the specified time (see Table 6).

TABLE 6
Time schedule for PK blood sampling during treatment course

| Trial | | | Time Point | Planned | |
|---|---|---|---|---|---|
| Periods | Visit | Day | [hh:min] | Time | Event |
| Treatment | 2 | 1 | Within 30 min before drug | −0:30 | PK Blood |
| period | | | admin. | | |
| | | | 0:00 | 0:00 | Drug admin. |
| | | | 0:30³ | 0:30 | PK Blood |
| | | | 1:00^{1,3} | 1:00 | PK Blood |
| | | | 2:00³ | 2:00 | PK Blood |
| | 3 | 4 (+3) | Within 5 min before drug | 71:55 | PK Blood |
| | | | admin. | | |
| | | | 0:00 | 72:00 | Drug admin. |
| | | | 1:00³ | 73:00 | PK Blood |
| | 4 | 29 (±3) | Within 5 min before drug | 671:55 | PK Blood |
| | | | admin. | | |
| | | | 0:00 | 672:00 | Drug admin. |
| | | | 1:00³ | 673:00 | PK Blood² |
| | 5 | 57 (±3) | Within 5 min before drug | 1343:55 | PK Blood² |
| | | | admin. | | |
| | | | 0:00 | 1344:00 | Drug admin. |
| | | | 1:00³ | 1345:00 | PK Blood |
| | EoT | 85 (±3) | Within 5 min before drug | 2015:55 | PK Blood |
| | | | admin. | | |
| | | | 0:00 | 2016:00 | Drug admin. |
| | | | 0:30³ | 2016:30 | PK Blood |
| | | | 1:00^{1,3} | 2017:00 | PK Blood |
| | | | 2:00³ | 2018:00 | PK Blood |
| | | | 4:00³ | 2020:00 | PK Blood |
| | | | 6:00³ | 2022:00 | PK Blood² |
| Follow-up | FUP 1 | 92 (±2) | One PK sample | 2184:00 | PK Blood |
| period | | | during the visit | | |

¹Post dose ECG should be performed either before or at least 10 min after PK blood sample.
²At selected sites, additional PK samples at planned times 673:00, 1343:55 and 2022:00 will be taken and acidified for CD 7949 quantification. Details on the handling and acidification of these specific samples will be provided in the laboratory manual.
³+/−15 minutes for post dose sample draws.

Patient should be instructed to bring all used and unused bottles to the clinic for each study visit. Site staff will inspect all the trial medication and also calculate compliance by capsule count. Site will destroy the empty bottles with documentation and re-dispense the remaining medication to the patient at each study visit. At the EoT visit all remaining used and unused bottles with trial medication will be collected from the patient. Study visits should be rescheduled if trial medication is not available for dosing the patient after the pre-dose PK sample is drawn.

If local regulations or site procedures or if the patient does not permit shipment of the trial medication directly to the patient, medication can be shipped to the study site. Randomization will still be completed at least 7 days before the scheduled visit 2 date to initiate trial medication shipment to the site. Site staff will dispense the trial medication to the patient.

Unscheduled supply of trial medication can be initiated by the study site in the IRT. Patient should contact the site if a need for additional medication supply arises.

A paper diary will be dispensed and collected at time points shown in Table 2. Patient will record the date and time of trial medication intake in the diary on the 3 days before EoT visit, and on the previous day for all other visits. Patients should be instructed to bring the completed diary for review by the site staff. Date and time of medication intake for the 3 days prior to EoT as well as the previous day for all other visits will be entered in the eCRF. Date and time of medication intake during the study visit will also be entered in the eCRF. A copy of the paper diary will be placed in the ISF.

For patients participating in the trial remotely via the DCT model, the MRN will visit the patient's home for the study visit and complete the trial procedures including inspection of the trial medication, providing instructions for administration of medication at home, administering trial medication at the specified time during the study visit, and review of the paper diary. Additional guidance on remote study visits will be provided in a separate DCT operational manual which will include instructions for handling used empty bottles and unused trial medication.

### COVID-19 Pandemic—Contingency Plan:

During the COVID-19 pandemic physical visits to the sites (for patients not participating in the DCT model) may need to be restricted to ensure patient safety. Based on a thorough assessment of the benefits and risks, the investigator may still decide to continue the trial treatment, after discussion with the sponsor.

### 2.1.5 Blinding and Procedures for Unblinding

### Blinding

The trial has a double-blind design. The randomization schemes and medication kit lists (i.e. the treatment information) will be handled according to the sponsor's Standard Operating Procedures (SOPs).

Below (Table 7) is a summary of the roles/function and the timing of unblinding.

TABLE 7
Unblinding protocol

| | |
|---|---|
| Role/Function | Timing of unblinding/receiving access to the treatment |
| | information (including rationale) |
| Subject/Participant, | This trial is blinded to the Subject/Participant and |
| Investigator/Site Staff | Investigator/Site Staff. |
| | The Subject/Participant's treatment information will be |
| | provided to the site after the completion of the study. |
| Sponsor - clinical trial | As needed, additional safety, efficacy, and PK/PD analysis |
| and project team. | may be performed during the course of the trial for which the |
| | database will be unblinded. |
| | Selected members of the trial/project team will receive |
| | access to the treatment information of the enrolled patients. |
| | Ongoing assessment of the trial data will support the clinical |
| | development of the TRPC6 inhibitor. |

Refer to Unblinding and Breaking the Code for rules of breaking the code for an individual or for all patients in emergency situations.

Unblinding and breaking the code Emergency unblinding will be available to the investigator via IRT. It must be used only in an emergency situation when the identity of the trial drug must be known to the investigator in order to provide appropriate medical treatment or otherwise assure safety of trial participants.

The reason for unblinding must be documented in the source documents and/or appropriate CRF page. If the patient is unblinded by the investigator, patient will have to be discontinued from the trial. Discontinued patients will complete the EoT and follow-up visits.

Due to the requirements to report Suspected Unexpected Serious Adverse Reactions (SUSARs), it may be necessary for a representative from BI's Pharmacovigilance group to access the randomization code for individual patients during trial conduct. The access to the code will only be given to authorized Pharmacovigilance representatives for processing in the PV database system and not be shared further.

### 2.1.6 Packaging, Labelling, and Re-Supply

The investigational medicinal products will be provided by BI or a designated contract research organization (CRO). They will be packaged and labelled in accordance with the principles of Good Manufacturing Practice. Re-supply (if necessary) will be managed via the IRT system. For details of packaging and the description of the label, refer to the ISF.

### 2.1.7 Storage Conditions

Drug supplies will be kept in their original packaging according to the recommended storage conditions on the medication label. Patients will be instructed to store the medication in a secure area. Site staff will train the patients on storage conditions. Patients will not maintain a temperature log.

If shipment of trial medication to the site becomes necessary, sites should store the medication in a secure limited access storage area according to the recommended storage conditions on the medication label and maintain a temperature log until the medication is dispensed to the patient. If the storage conditions are found outside the specified range, procedure described in the ISF has to be followed and a Clinical Research Associate (CRA) should be contacted immediately.

### 2.1.8 Drug Accountability

The patient will receive the investigational drug delivered by the sponsor when the following requirements are fulfilled at the study site:
Approval of the clinical trial protocol by the Institutional Review Board (IRB)/ethics committee,
Availability of a signed and dated clinical trial contract between the sponsor or delegate and the investigational site,
Approval/notification of the regulatory authority, e.g. competent authority,
Availability of the curriculum vitae of the Principal Investigator,
Availability of a signed and dated clinical trial protocol,
Availability of the proof of a medical license for the Principal Investigator,
Availability of Food and Drug Administration (FDA) Form 1572 (if applicable),
Study site has obtained a signed informed consent from the patient.

Investigational drugs are not allowed to be used outside the context of this protocol. They must not be forwarded to other investigators or clinics. Patients should be instructed to return all unused investigational drug.

The investigator or designee must maintain records of the product's delivery to the patient, the use by each patient, and the return to the sponsor or warehouse/drug distribution center or alternative disposal of unused products. If applicable, the sponsor or warehouse/drug distribution center will maintain records of the disposal. These records will include dates, quantities, batch/serial numbers, expiry (‘use-by’) dates, and the unique code numbers assigned to the investigational medicinal product and trial patients. The investigator or designee will maintain records that document adequately that the patients were provided the doses specified by the Clinical Trial Protocol and reconcile all investigational medicinal products the patient received from the sponsor. At the time of return to the sponsor and/or appointed CRO, the investigator or designee must verify that all unused or partially used drug supplies have been returned by the patient and that no remaining supplies are in the investigator's possession.

### 2.2 Other Treatments, Emergency Procedures, Restrictions

### 2.2.1 Other Treatments and Emergency Procedures

There are no special emergency procedures to be followed in this trial.

### 2.2.2 Restrictions

Table 8 shows restrictions regarding concomitant treatment

TABLE 8

| Medication or class of medications | Restriction needs and time |
|---|---|
| Strong inhibitors and strong inducers of | Not allowed 1 week or 5 half-lives |
| CYP3A4/5; immunosuppressive agents, | (whichever is longer) prior to randomization |
| drugs with UGT1A4 activity, drug which are | through 5 days after EOT |
| known P-gp substrates of narrow | |
| therapeutic window, and OCT2, MATE1, or | |
| MATE2-K substrates. | |
| Investigational device or drug | Not allowed 30 days or 5 half-lives |
| | (whichever is longer) prior to |
| | randomization through 5 days after EOT |
| Agents known to prolong the QT interval | Not allowed 1 week or |
| | 5 half-lives (whichever is longer) prior to |
| | randomization through 5 days after EOT |
| Systemic corticosteroids | Dose must be stable for at least 4 weeks |
| | prior to screening visit. Dose should not |
| | change during the screening and |
| | treatment period unless the investigator |
| | feels a dose change is necessary to |
| | ensure the patient's safety. |

Additional information, including a list of drugs that should be avoided, is provided in the ISF for the following classes of medications: immunosuppressive agents, strong inhibitors/inducers of CYP3A4/5, UGT1A4, drugs which are known P-gp substrates of narrow therapeutic window, and OCT2, MATE1, or MATE2-K substrates as well as agents known to prolong the QT interval.

Medications listed under the exclusion criteria are not allowed during the trial. When possible, use of restricted medications and impact on treatment discontinuation should be discussed with the sponsor. There are no restrictions for trial participant to receive vaccination for COVID-19 during or after the study period.

Restrictions on Diet and Lifestyle Patients should avoid high protein, high salt diet. Avoidance of dietary protein load and strenuous exercise is especially important within 24 hours prior to the start and until the completion of the 24-hour urine collections.

### Contraception Requirements

### Female Patients

WOCBP and their male sexual partner must use two medically approved methods of birth control during the treatment period and for a period of at least 5 days after last trial drug intake. Male partner of a WOCBP trial participant who is able to father a child must use a condom.

WOCBP (trial participant) must use a highly effective method of birth control per ICH M3 (R²) that results in a low failure rate of less than 1% per year when used consistently and correctly. Birth control methods with low user dependency, as indicated with an asterisk (*) below are preferable.
Combined (estrogen and progestogen containing) hormonal birth control that prevents ovulation (oral, intravaginal, transdermal)
Progestogen-only hormonal birth control that prevents ovulation (oral, injectable, implantable*)
Intrauterine device (IUD)* or intrauterine hormone-releasing system (IUS)*
Bilateral tubal occlusion*

Acceptable methods of birth control will include abstinence from male-female sex or having a vasectomized partner, provided that partner is the sole sexual partner of the trial participant who is a WOCBP, and that the vasectomized partner has received medical assessment of the surgical success.

Abstinence from male-female sex is defined as being in line with the preferred and usual lifestyle of the patient. Periodic abstinence e.g. calendar, ovulation, symptothermal, post-ovulation methods; declaration of abstinence for the duration of exposure to study drug; and withdrawal are not acceptable.

Since the TRPC6 inhibitor is not expected to lead to a clinically relevant reduction in the exposure of oral contraceptives due to an increased metabolism via enzyme induction, oral contraceptives are allowed in this trial.

### 2.3 Treatment Compliance

Patients are requested to bring all remaining trial medication including empty bottles with them when attending visits. Based on capsule counts, treatment compliance will be calculated as shown in the formula below.

$$Treatment compliance ( \% ) = \frac{Number of capsules actually taken \times 100}{Number of capsules should have been taken as directed by the investigator}$$

Compliance will be verified by the CRA authorized by the sponsor or delegate. The target for medication compliance should be 100%. If patient is non-compliant, site staff will explain to the patient the importance of treatment compliance. Randomized patients will not be discontinued from the trial for poor medication compliance without prior discussion with the Clinical Trial Manager (CT Manager) appointed by the sponsor.

For patients participating in the trial remotely via the DCT model, compliance will be calculated by the MRN, and all unused trial medication and empty bottles will be collected by the MRN and returned to the study site or an alternative location for disposal. Shipment schedule and guidelines will be described in the DCT operational manual. The investigator or designee must verify that all unused trial medication has been returned by the patient and that no remaining supplies are at the patient's home.

### 3. Assessments

### 3.1 Assessment of Efficacy

### 3.1.1 Measures of Proteinuria

The primary endpoint is patients achieving at least 25% reduction in UPCR from 24-hour urine relative to baseline after 12 weeks of treatment. The baseline UPCR will be from the average of two 24-hour urine samples collected before visit 2.

Secondary and further endpoints are listed in under Secondary Endpoints and Further Endpoints., respectively, and they will be assessed as follows:
Change in UPCR relative to visit 3 at week 12: the samples will be from the 24-hour urine samples collected at visits 3 and 12.
Change in UPCR relative to baseline at week 13: the samples will be the 24-hour urine samples collected at baseline and week 13.
Change in 24-hour urinary protein excretion relative to baseline at week 12.
Change in UACR relative to baseline (visit 2, first dose) at weeks 4, 8, 12 and 13: spot urine samples will be used for this analysis.
Change in UPCR relative to baseline (visit 2, first dose) at weeks 4, 8, 12, and 13: spot urine samples will, be used for this analysis.

For additional information on urine sample collections and the time points, see the Visit Schedule and Table 2.

3.1.2 eGFR Assessments

For the assessment of the further endpoint, eGFR measurements collected at visit 2 (before first dose) will be used as baseline and compared to the measurements at week 12 and 13. The Chronic Kidney Disease-Epidemiology Collaboration (CKD-EPI) formula based on serum cystatin C will be used for efficacy analysis.

### 3.2 Assessment of Safety

### 3.2.1 Physical Examination

A complete physical examination will be performed at the time points specified in Table 2. It includes at a minimum general appearance, neck, lungs, cardiovascular system, abdomen, extremities, and skin. Measurement of height and body weight will be performed at the time points specified in Table 2. The results must be included in the source documents available at the site.

For patients participating in the trial remotely via the DCT model, a physical examination per protocol will be performed at the patient's home at the time points specified in the study protocol. The investigator or a designee (as indicated in the site trial staff list) will supervise the physical examination remotely. A MRN will facilitate the physical examination in the patient's home.

For patients participating in the trial remotely, if during the physical examination (PE) or at any time during the trial it is deemed that the patient requires in-person follow-up, the patient will be advised to visit the study site or their healthcare provider or a local facility for consultation. When applicable, the study site staff will contact the local healthcare facility to obtain the medical records.

### 3.2.2 Vital Signs

Vital signs will be evaluated at the time points specified in Table 2, prior to blood sampling. This includes systolic and diastolic blood pressure and pulse rate (electronically or by palpation count for 1 minute) in a seated position after 5 minutes of rest. Assessment for uncontrolled hypertension is completed at Visit 1 only. Three blood pressure measurements will be taken approximately 2 minutes apart after the patient has rested quietly and is in a seated position for at least 5 minutes. The average of the last two measurements for the triplicate systolic blood pressure measurements should be <160 mmHg for assessment of exclusion criteria 5 at the screening visit. For subsequent visits, triplicate measurements are needed only if the first reading is >160 mmHg, the average of the last two measurements are taken as the final reading. The results must be included in the source documents available at the site.

### 3.2.3 Safety Laboratory Parameters

Safety laboratory parameters to be assessed are listed in Table 9. Sampling time points are provided in Table 2.

All analyses will be performed by a central laboratory, and the respective reference ranges will be provided in the laboratory manual. Instructions regarding sample collection, sample handling, processing, and shipping are provided in the laboratory manual.

If central lab services or the lab kits provided by the central lab are not available at the study site, safety labs may be done at a local lab. The results of the lab tests should be entered in the eCRF. Please note the local labs should be used only if it becomes necessary, and the CT Manager should be informed.

For patients participating in the trial remotely via the DCT model, laboratory kits will be made available at the patient's home for the respective visits. The MRN will collect, process, and ship lab samples to the central laboratory or the study site (when applicable). Additional information will be provided in the DCT operations manual.

Patients do not have to be fasted for the blood sampling for the safety laboratory.

The central laboratory will send laboratory reports to the investigator. It is the responsibility of the investigator to evaluate the reports. Clinically relevant abnormal findings as judged by the investigator will be reported as AEs.

In case the criteria for hepatic injury are fulfilled, a number of additional measures will be performed (see the Definition of AEs and the DILI Checklist provided in the electronic data capture (EDC) system). The amount of blood taken from the patient concerned will be increased due to this additional sampling for DILI assessments. The central laboratory will transfer the data to the sponsor periodically.

For assessment of eGFR exclusion criterion, the CKD-EPI formula based on serum creatinine and serum cystatin C will be used. The serum cystatin C-based CKD-EPI formula will be used for the calculation of eGFR in the evaluation of efficacy. For sensitivity analysis, eGFR will also be calculated using CKD-EPI formula based on serum creatinine. Inker L A et al., “CKD-EPI Investigators. Estimating glomerular filtration rate from serum creatinine and cystatin C,” N Engl J Med 2012; 367(1):20-29.

TABLE 9
Safety laboratory tests.

| Functional lab group | Test name |
|---|---|
| Haematology | Haematocrit |
| | Haemoglobin |
| | Red blood cells (RBC) |
| | White blood cells (WBC) |
| | Platelet count |
| | MCV |
| | MCH |
| | MCHC |
| | RDW |
| Automatic WBC differential | Neutrophils total |
| (relative and absolute) | Lymphocytes total |
| | Eosinophils |
| | Basophils |
| | Monocytes |
| | Lymphocytes |
| Manual differential WBC | Polymorphonuclear neutrophils (segs), band neutrophils |
| (if automatic differential WBC is | (stabs), eosinophils, basophils, monocytes, lymphocytes |
| abnormal) | |
| Coagulation | Activated partial thromboplastin time (aPTT) |
| | Prothrombin Time (PT) |
| | International Normalised Ratio (INR) |
| Enzymes | Aspartate aminotransferase (AST) |
| | Alanine aminotransferase (ALT) |
| | Alkaline phosphatase (ALP) |
| | Gamma-glutamyl transferase (GGT) |
| | Creatine Kinase (CK) |
| | Creatine kinase - MB fraction (CK-MB¹) |
| | Lactate dehydrogenase |
| | Lipase |
| | Amylase |
| | Troponin I¹ |
| Substrates | Glucose |
| | HbA1c (at screening and EoT) |
| | Creatinine (enzymatic method)² |
| | Cystatin C |
| | eGFR - CKD-EPI formula based on serum creatinine |
| | and cystatin C³ |
| | eGFR - CKD-EPI based on serum creatinine |
| | eGFR - CKD-EPI based on serum cystatin C⁴ |
| | Urea |
| | Uric Acid |
| | Total bilirubin |
| | Direct bilirubin |
| | Total protein |
| | Triglycerides |
| | Albumin |
| | Globulin |
| | Albumin/Globulin ratio |
| | C-Reactive Protein (CRP) |
| | Total cholesterol |
| Infectious serology⁵ | Hepatitis B surface antigen |
| | Hepatitis C antibodies |
| | HIV-1/2 combination |
| Electrolytes | Calcium |
| | Sodium |
| | Potassium |
| | Magnesium |
| | Chloride |
| | Phosphate |
| | Bicarbonate |
| | (calculated) anion gap |
| Urinalysis | Urine nitrite |
| | Urine protein |
| | Urine glucose |
| | Urine ketone |
| | Urine bilirubin |
| | Urine Blood |
| | Urine leukocyte esterase |
| | Urine pH |
| | Specific gravity |
| | Urine drug screen (screening visit only) |
| | Cannabis |
| | Cocaine |
| | Benzodiazepine |
| | Amphetamines |
| | Barbiturates |
| | Methadone |
| | Opiates |
| Urine sediment (microscopic | Only positive findings will be reported (e.g. presence of |
| examination if erythrocytes, | sediment bacteria, casts in sediment, squamous |
| leukocytes, nitrate or protein are | epithelial cells, erythrocytes, leukocytes). |
| abnormal in urine) | |
| Serum Pregnancy test (only for | Human Serum Chorionic Gonadotropin |
| female participants of childbearing | |
| potential) at Visit 1, and if urine | |
| pregnancy test is positive at other | |
| visits. | |
| Urine pregnancy test | Human Serum Chorionic Gonadotropin |

¹If initial CK is elevated, re-test CK with CK-MB and troponin I.
²Reported to the investigator only up to visit 2.
³eGFR based on serum creatinine and cystatin C will be reported to the investigator only up to visit 2.
⁴eGFR based on serum cystatin C will be reported to the investigator from visit 2 onwards.
⁵Only at screening visit.

### 3.2.4 Electrocardiogram

Centralized ECG services will be provided by an external vendor. Standardized equipment and a quick guide will be provided by the vendor. ECGs should be collected according to the study-specific recommendations, using the standardized equipment provided by the vendor.

The 12-lead ECGs will be recorded at the time points shown in the Table 2. ECGs should be recorded or at least 10 minutes after before blood samples are drawn. Patients should be supine for approximately 5 to 10 minutes before ECG collection. Patients should remain supine, but awake, during the ECG collection process.

After the screening visit, the investigator must review the ECG results from central reading to ensure that patient is eligible to participate in the study. Starting at visit 2, ECGs will be recorded at two time points at each study visit: pre-dose and at 1 to 2 hours post-dose. ECGs may be repeated for quality or safety reasons.

ECG recordings will be transmitted electronically to a vendor for central reading. ECGs will be centrally evaluated and rated as normal, abnormal, or unable to evaluate, and the results will be sent to the study site. The investigator should review the report from central reading. If the ECG is rated as abnormal, the investigator will determine if the abnormal findings are clinically significant. The investigator will have the responsibility to follow up with the patient if there are any clinically significant findings in the ECG report.

At the screening visit, ECGs are done in triplicate (3 single ECGs recorded within 180 seconds). The QTcF value used to check eligibility at the screening visit is the average of the three recordings. Any pre-existing conditions should be recorded as baseline conditions.

Pre-dose ECGs at the first dosing visit should be evaluated by the investigator before the patient receives the first dose. If abnormalities are observed by the investigator in the ECG readings at the first dosing visit, the investigator may wait until the results from central reading are available, and the first dosing visit may be rescheduled.

After the first dose is taken at visit 2, ECG will be done 1 to 2 hours post-dose. In each of the remaining study visits until EoT, ECG will be recorded pre-dose and 1 to 2 hours post-dose. At the FUP1 visit, ECG will be recorded only once. ECGs will not be recorded at EoS visit.

After the patient receives the first dose, if a clinically significant increase in the QTcF interval from baseline (defined as visit 2, pre-dose measurement) or any other clinically significant quantitative or qualitative change from baseline is identified the investigator will assess the symptoms (e.g., palpitations, near syncope, or syncope) and decide if the patient will continue in the trial. The investigator must also check if the patient meets any of the treatment discontinuation criteria. Any new pathological findings (including clinically relevant abnormal ECG findings) or deterioration of previous findings observed during the trial will be recorded as AEs or SAEs, and should be followed up and/or treated as medically appropriate per local standards.

Although the ECGs are transmitted to the vendor for central reading, the investigator has the responsibility to complete an initial review of the ECG recordings the same day as the study visit. At any time during the trial, the investigator may decide to place a hold on further dosing of the patient if there is an indication of any new pathologic abnormalities in the ECG, and would prefer to wait until the results from the central reading are available.

All ECGs that are read in the central location will be stored in the vendor's database and will be transmitted to the sponsor periodically.

For patients participating in the trial remotely via DCT, ECGs are completed at the patient's home. The MRN will upload the ECGs to the DCT Platform (when applicable), and they will be available for the investigator or the site staff for review. If necessary, the MRN should consult with the investigator or designee before dosing the patient.

### 3.2.5 Other Safety Parameters

### Ocular Safety Assessments

Eye exams including the evaluation of cataract by slit lamp will be performed by an ophthalmologist or an optometrist in both eyes to evaluate the presence of lens disorders and cataract during the screening period. Results from the eye exams must be available to the investigator before the patient is randomized in the IRT and trial medication shipment is initiated. The eye assessments will be repeated at EoT, and 30 days after last dose of trial medication (EoS) to monitor eye health and to observe any changes from baseline. The eye exams do not have to be completed on the same day of the study visits. At study entry, eye exams can be completed during the screening period. For EoT and EoS visits, patients should try and complete the eye exams within the protocol allowed window (±3 days) for the respective visits. Eye exams should be completed at the study site or at another healthcare or eye care facility.

Eye assessments will be performed according to the Eye Examination Worksheet provided by the sponsor. All cataracts will be graded using LOCS Ill. A copy of the worksheet will be available in the ISF. The results from the eye exams should be entered in a separate eCRF. Safety related findings will be reported as AEs if applicable. If documented medical history of cataract surgery in both eyes is available at the time of screening, eye assessments are not required, and patient will not undergo eye exams during the screening period, EoT, and EoS. If it is confirmed at the screening visit (by slit lamp exam) that the patient had cataract surgery in both eyes, eye assessments are not required at EoT and EoS.

Patients participating in the trial remotely via the DCT model will complete the eye exams at a facility referred to by the investigator or patient could complete these exams at a local healthcare facility close to the patient's home. Results from the eye exams must be sent to the study site.

### 3.2.6 Assessment of Adverse Events

### Definitions of AEs

### 3.2.6.1.1 Adverse Event

An AE is defined as any untoward medical occurrence in a patient or clinical investigation subject administered a medicinal product and which does not necessarily have to have a causal relationship with this treatment.

An AE can therefore be any unfavorable and unintended sign (including an abnormal laboratory finding), symptom, or disease temporally associated with the use of a medicinal product, whether or not considered related to the medicinal product.

The following should also be recorded as an AE in the CRF and BI SAE form (if applicable):
Worsening of the underlying disease or of other pre-existing conditions.
Changes in vital signs, ECG, physical examination and laboratory test results, if they are judged clinically relevant by the investigator.

If such abnormalities already exist prior to trial inclusion, they will be considered as baseline conditions and should be collected in the eCRF only.

### 3.2.6.1.2 Serious Adverse Event

A serious adverse event (SAE) is defined as any AE, which fulfils at least one of the following criteria:
results in death,
is life-threatening, which refers to an event in which the patient was at risk of death at the time of the event; it does not refer to an event that hypothetically might have caused death if more severe,
requires inpatient hospitalization or prolongation of existing hospitalization
results in persistent or significant disability or incapacity,
is a congenital anomaly/birth defect,
is deemed serious for any other reason if it is an important medical event when based on appropriate medical judgement which may jeopardize the patient and may require medical or surgical intervention to prevent one of the other outcomes listed in the above definitions. Examples of such events are intensive treatment in an emergency room or at home for allergic bronchospasm, blood dyscrasias or convulsions that do not result in hospitalization or development of dependency or abuse.

### 3.2.6.1.3 AEs Considered “Always Serious”

In accordance with the European Medicines Agency initiative on Important Medical Events, BI has set up a list of AEs, which by their nature, can always be considered to be “serious’ even though they may not have met the criteria of an SAE as defined above.

The latest list of “Always Serious AEs” can be found in the EDS system. A copy of the latest list of “Always Serious AEs” will be provided upon request. These events should always be reported as SAEs.

Cancers of new histology and exacerbations of existing cancer must be classified as a serious event regardless of the time since discontinuation of the drug and must be reported as described in AE Collection and AE Reporting to Sponsor and Timelines.

### 3.2.6.1.4 Adverse Events of Special Interest

The term adverse events of special interest (AESI) relates to any specific AE that has been identified at the project level as being of particular concern for prospective safety monitoring and safety assessment within this trial, e.g. the potential for AEs based on knowledge from other compounds in the same class. AESIs need to be reported to the sponsor's Pharmacovigilance Department within the same timeframe that applies to SAEs.

The following are considered as AESIs:

### Potential Severe DILI

A potential severe Drug Induced Liver Injury (DILI) that requires follow-up defined by the following alterations of hepatic laboratory parameters:
an elevation of AST and/or ALT≥3 fold upper limit normal (ULN) combined with an elevation of total bilirubin≥2 fold ULN measured in the same blood draw sample, or in samples within 30 days of each other, or
ALT and/or AST elevations 10-fold ULN.

These lab findings constitute a hepatic injury alert and the patients showing these lab abnormalities need to be followed up according to the “DILI checklist” provided in the EDC.

In case of clinical symptoms of hepatic injury (icterus, unexplained encephalopathy, unexplained coagulopathy, right upper quadrant abdominal pain, etc.) without lab results (ALT, AST, total bilirubin) available, the investigator should make sure these parameters are analyzed, if necessary, in an unscheduled blood test. Should the results meet the criteria of hepatic injury alert, the procedures described in the DILI checklist should be followed

### 3.2.6.1.5 Intensity (Severity) of AEs

The intensity (severity) of the AE should be judged based on the following:
Mild: Awareness of sign(s) or symptom(s) that is/are easily tolerated.
Moderate: Sufficient discomfort to cause interference with usual activity.
Severe: Incapacitating or causing inability to work or to perform usual activities.

### 3.2.6.1.6 Causal Relationship of AEs

Medical judgement should be used to determine the relationship between the adverse event and the BI investigational compound, considering all relevant factors, including pattern of reaction, temporal relationship, de-challenge or re-challenge, confounding factors such as concomitant medication, concomitant diseases and relevant history.

Arguments that may suggest that there is a reasonable possibility of a causal relationship could be:
The event is consistent with the known pharmacology of the drug.
The event is known to be caused by or attributed to the drug class.
A plausible time to onset of the event relative to the time of drug exposure.
Evidence that the event is reproducible when the drug is re-introduced.
No medically sound alternative aetiologies that could explain the event (e.g. pre-existing or concomitant diseases, or co-medications).
The event is typically drug-related and infrequent in the general population not exposed to drugs (e.g. Stevens-Johnson syndrome).
An indication of dose-response (i.e. greater effect size if the dose is increased, smaller effect size if dose is reduced).

Arguments that may suggest that there is no reasonable possibility of a causal relationship could be:
No plausible time to onset of the event relative to the time of drug exposure is evident (e.g. pre-treatment cases, diagnosis of cancer or chronic disease within days/weeks of drug administration; an allergic reaction weeks after discontinuation of the drug concerned).
Continuation of the event despite the withdrawal of the medication, taking into account the pharmacological properties of the compound (e.g. after 5 half-lives). Of note, this criterion may not be applicable to events whose time course is prolonged despite removing the original trigger.
Additional arguments amongst those stated before, like alternative explanation (e.g. situations where other drugs or underlying diseases appear to provide a more likely explanation for the observed event than the drug concerned).
Disappearance of the event even though the trial drug treatment continues or remains unchanged.

### Adverse Event Collection and Reporting

### 3.2.6.1.7 AE Collection

The investigator shall maintain and keep detailed records of all AEs in the patient files.

The following must be collected and documented on the appropriate CRF(s) by the investigator:
From signing the informed consent onwards until the follow-up visit: all AEs (serious and non-serious) and all AESIs.
After follow-up-visit 1 until the individual patient's end of trial: cancers of new histology and exacerbations of existing cancer, all trial drug related SAEs and all trial drug related AESIs.
After the individual patient's end of trial: the investigator does not need to actively monitor the patient for new AEs but should only report any occurrence of cancer and trial treatment related SAEs and trial treatment related AESIs of which the investigator may become aware of by any means of communication, e.g. telephone call. Those AEs should be reported on the BI SAE form, but not on the CRF.

### 3.2.6.1.8 AE Reporting to the Sponsor and Timelines

The investigator must report SAEs, AESIs, and non-serious AEs which are relevant for the reported SAE or AESI, on the BI SAE form to the sponsor's unique entry point within 24 hours of becoming aware of the event. Country specific process will be specified in the ISF. The same timeline applies if follow-up information becomes available. In specific occasions, the investigator could inform the sponsor upfront via telephone. This does not replace the requirement to complete the BI SAE form.

With receipt of any further information to these events, a follow-up SAE form has to be provided. For follow-up information the same rules and timeline apply as for initial information. All (S)AEs, including those persisting after individual patient's end of trial must be followed up until they have resolved, have been assessed as “chronic” or “stable”, or no further information can be obtained.

### 3.2.6.1.9 Pregnancy

In rare cases, pregnancy might occur in a clinical trial. Once a patient has been enrolled in the clinical trial and has taken trial medication, the investigator must report any drug exposure during pregnancy in a trial participant immediately (within 24 hours) by means of Part A of the Pregnancy Monitoring Form to the sponsor's unique entry point.

The outcome of the pregnancy associated with the drug exposure during pregnancy must be followed up and reported to the sponsor's unique entry point on the Pregnancy Monitoring Form for Clinical Studies (Part B). The ISF will contain the Pregnancy Monitoring Form for Clinical Studies (Part A and B).

As pregnancy itself is not to be reported as an AE, in the absence of an accompanying SAE and/or AESI, only the Pregnancy Monitoring Form for Clinical Studies and not the SAE form is to be completed. If there is an SAE and/or AESI associated with the pregnancy an SAE form must be completed in addition.

### 3.2.6.1.10 Additional Safety Monitoring

In addition to the standard AE and SAE reporting, additional information will be collected in a separate eCRF on the following:
Acute kidney injury
Cataract

### 3.3 Drug Concentration Measurements and Pharmacokinetics

### 3.3.1 Assessment of Pharmacokinetics

Blood samples for PK will be collected according to planned dates and times provided in Table 6. The date and clock times of drug administration and pharmacokinetic sampling will be recorded in the eCRF. The actual sampling times will be used for determination of pharmacokinetic parameters as defined in Secondary Endpoints. PK samples collected from this study will be also used for population PK and/or PK/PD analyses.

### 3.3.2 Methods of Sample Collection

For the quantification of TRPCi plasma concentrations, blood samples will be collected at time points indicated in Table 6 and Table 2. The actual sampling times and time of dosing will need to be recorded in the eCRF. At selected sites, additional samples will be taken for the exploratory investigation of metabolites. These additional samples will be acidified as described in the laboratory manual.

Plasma samples should be shipped to the central laboratory preferably on the same day of collection. Samples should be stored at approximately −20° C. or below. Detailed instructions on sampling, preparation, processing, shipment and storage are provided in the laboratory manual.

After completion of the trial the plasma samples may be used for further methodological or exploratory investigations, e.g. for stability and metabolite testing. However, only data related to the analyte and/or its metabolites will be generated by these additional investigations. The study samples will be discarded after completion of the additional investigations but not later than 5 years after the final study report has been signed.

### 3.3.3 Analytical Determinations

The TRPC6 inhibitor concentrations in plasma will be determined by a validated liquid chromatography tandem mass spectrometry (LC-MS/MS) assay. All details of the analytical method will be available prior to the start of sample analysis. Metabolite concentrations in plasma will be determined by an exploratory assay. Results will be reported separately.

All samples from subjects on active drug will be analyzed. From subjects on placebo, only one time point will be analyzed in order to demonstrate absence of drug. In case there are quantifiable drug concentration, all PK samples from the placebo subject in question would be analyzed.

The analysis will be performed under the responsibility of Drug Metabolism and Pharmacokinetics, Boehringer Ingelheim Pharma GmbH & Co. KG, Biberach, Germany at a suitable contract research organization. The bioanalyst will be unblinded during sample analysis to allow pharmacokinetic analyses during the course of the trial as described herein.

### 3.3.4 Pharmacokinetic—Pharmacodynamic Relationship

Exploratory analysis of PK-PD relationship will be performed with C_{pre,ss}, C_{max,ss} and AUC_{0-6,ss} of the TRPC6 inhibitor (if feasible; from week 12) and the following endpoints/biomarkers,
The proportion of patients (%) achieving at least 25% reduction 24-hour UPCR relative to baseline at week 12.
Change in UPCR from baseline.
Mechanistic urinary biomarkers reflective of podocyte health:
   podocin [mRNA] creatinine ratio (UPodCR)
   nephrin [mRNA] creatinine ratio (UNephCR)
   podocin [mRNA] nephrin [mRNA] ratio (UPNR)
Mechanistic urinary biomarkers reflective of drug target modulation:
   TPRPC6 mRNA
   NFAT mRNA
   Further downstream markers in the calcineurin-NFAT pathway.

In addition, the relationship between the TRPC6 inhibitor plasma concentrations and ECG variables (e.g. HR, QTcF, QTc) will be explored. Details will be described in the TSAP.

### 3.4 Assessment of Biomarkers

In order to characterize the effect of the TRPC6 inhibitor in patients with chronic kidney disease/FSGS, a number of biomarker panels will be analyzed that represent the key mechanisms of kidney pathophysiology, such as inflammation, fibrosis, tubulo-interstitial injury, oxidative stress, glomerular injury, and endothelial dysfunction. It is planned to utilize the results of these measurements for comparison of the TRPC6 inhibitor with other compounds targeting chronic kidney disease or data from the literature or for use in pharmacometric modelling. Plasma, serum and urine samples will be collected as per Table 2. The full list of planned exploratory biomarkers is provided in Table 10.

TABLE 10
Exploratory Biomarkers

| Category | Biomarkers |
|---|---|
| Exploratory biomarkers in urine and/or | Urine: |
| serum planned to be assessed at BI or a | Creatinine (for normalization) |
| CRO authorized by BI: | Nephrin |
| | Podocin |
| | Podocalyxin |
| | Connective tissue growth factor |
| | Fibronectin |
| | 8-Hydroxydesoxyguanosine |
| | 8-Isoprostane |
| | RNA profiling from urine sediment, including |
| | podocin and nephrin, TPRPC6 and NFAT |
| | mRNA |
| | RNA profiling from urinary extracellular |
| | vesicles |
| | Serum: |
| | Connective tissue growth factor |
| Exploratory biomarkers in plasma planned to | Plasma: |
| be assessed at BI or a CRO authorized by | Adiponectin |
| BI using a custom multi-analyte panel. Due | Angiopoietin 2 |
| to the assay format, results may be | C-Reactive Protein |
| generated for additional biomarkers that may | E-Selectin |
| also be relevant for chronic kidney | Fibroblast Growth Factor 23 |
| disease/FSGS. | Intercellular Adhesion Molecule 1 |
| | N-terminal Prohormone of Brain Natriuretic |
| | Peptide |
| | Platelet-Derived Growth Factor BB |
| | Tumor Necrosis Factor Receptor 1 |
| | Tumor Necrosis Factor receptor 2 |
| | Vascular Cell Adhesion Molecule-1 |
| | von Willebrand Factor |
| Exploratory biomarkers in urine planned to | Urine: |
| be assessed at BI or a CRO authorized by | Adiponectin |
| BI using a custom multi-analyte panel. Due | Beta-2-Microglobulin |
| to the assay format, results may be | Cellular Fibronectin |
| generated for additional biomarkers that may | Collagen IV |
| also be relevant for chronic kidney | C-Reactive Protein |
| disease/FSGS. | Cystatin C |
| | Epidermal Growth Factor |
| | Fatty Acid-Binding Protein, liver |
| | Fibrinogen |
| | Fibroblast Growth Factor 23 |
| | Growth-Regulated Alpha Protein |
| | Kidney Injury Molecule-1 |
| | Monocyte Chemotactic Protein 1 |
| | Neutrophil Gelatinase-Associated Lipocalin |
| | Tissue Inhibitor of Metalloproteinases 1 |

### 5.4.1 Biochemical Biomarkers

### Methods of Sample Collection

The time points and methods for collection of samples for biomarkers are described below. All blood and urine samples will be discarded one year after the last patient has completed the trial.

### Blood Sampling

All blood samples for measurements in plasma or serum will be taken from an antecubital or forearm vein by means of either an indwelling venous catheter or by venipuncture with a metal needle at the time points indicated in the Table 2.

For the measurement of exploratory biomarkers, at each time point indicated in Table 2, blood will be drawn into a potassium ethylenediaminetetraacetic acid (K-EDTA)-anticoagulant blood drawing tube, and into a serum gel tube. Details of sample processing, including preparation of aliquots will be provided in a study-specific laboratory manual.

### Urine Sampling

For exploratory biomarkers, spot urine will be collected during the study visits as designated in Table 2. If the visits are conducted remotely, urine biomarker samples will not be collected. For the measurement of the mechanistic biomarkers of podocyte health such as podocin and nephrin mRNA and drug target modulation such as TRPC6 mRNA (see Pharmacodynamic Endpoints and Table 10, urine aliquots from spot urine will be centrifuged to obtain urine sediment. The resulting supernatant will be used for the isolation of extracellular vesicles and molecular profiling of their cargo.

Details of sample processing, including centrifugation where required, preparation of aliquots, and the sequence in which the aliquots will be prepared will be described in the laboratory manual.

### Analytical Determinations

All measurements are considered exploratory biomarkers and measured using fit-for-purpose validated method. Apart from routine central laboratory tests, all analytical methods will be described in detail along with the measurement results in a separate biomarker report.

All analyses will be performed at Boehringer Ingelheim Pharma GmbH & Co. KG, Biberach, Germany or a CRO duly authorized by BI.

### 3.4.1 Pharmacogenomics Biomarkers

Pharmacogenomics investigates genetic variations to explain and to predict an individual's response to drugs. Therefore, a blood sample for pharmacogenomic testing will be taken from each subject. In case of unexplainable variability of PK or PD parameters, DNA may be extracted from these samples and used for exploratory analysis of variants of genes with known association with FSGS, in particular TRPC6, and/or genes involved in absorption, distribution, metabolism and excretion of drugs.

It is not intended to include these data in the clinical trial report. However, the data may be part of the report if necessary. All DNA samples will be destroyed one year after the last patient has completed the trial.

Detailed instructions for pharmacogenomics sampling, handling, and shipment of samples will be provided in the laboratory manual.

Methods and Timing of Sample Collection One blood sample will be taken from an arm vein into a PAXgene blood DNA drawing tube, preferably at Visit 2.

### Analytical Determinations

DNA will be extracted from blood samples according to standard molecular genetics methods and analyzed by Drug Metabolism Enzymes and Transporters (DMET) analysis or other standard genotyping technologies.

### 3.5 Biobanking

Not applicable.

### 3.6 Other Assessments

Not applicable.

### 3.7 Appropriateness of Measurements

All measurements except for the exploratory biomarker measurements performed during this trial are standard measurements and will be performed in order to monitor patients' safety and to determine pharmacokinetic and pharmacodynamic parameters. Sampling for the exploratory biomarkers is not associated with any additional risks. The risks related to eye exams are minimal.

The scheduled procedures and measurements will allow monitoring of changes in vital signs, standard laboratory values, eye health, and ECG parameters that might occur as a result of administration of study drug. The safety assessments are standard, and are accepted for evaluation of safety and tolerability, and are widely used in clinical trials. The pharmacokinetic parameters and measurements outlined are generally used for assessments of drug exposure. Approximately 200 mL (14 tablespoons) of blood will be collected from a patient during the course of the study.

### 4. Investigational Plan

### 4.1 Visit Schedule

The trial consists of a screening period, treatment period, and a follow-up period. Following the screening period, patients will be randomized (prior to visit 2) to one of the four treatment arms. The treatment period is followed by a 30-day follow-up period which consists of 2 follow-up visits. The 2^{nd} follow-up visit which is also the EoS visit will be completed by telephone except for the eye exams. Visit schedule and trial procedures are provided in Table 2 and Table 11. Schedule for PK sampling is presented in Table 6.

TABLE 11
Scheduled telephone visits.

| Phone visits¹ | 3A | 4A | 5A² |
|---|---|---|---|
| Week | 2 | 6 | 10 |
| Days from first dose | 15 | 43 | 71 |
| Window (days) | ±3 | ±3 | ±3 |
| Concomitant therapy | X | X | X |
| All AEs³/SAEs/AESIs | X | X | X |

Footnotes:
¹Patients will be contacted by telephone. If there is any follow-up needed, an unscheduled visit may be scheduled. Patients should be reminded to be compliant with the intake of trial medication.
²Patient should be reminded to collect the 24-hour urine before EOT visit.
³Please see footnote #26 for Table 2.

Patients should make all efforts to complete the trial which includes the 2 follow-up visits. Investigators should encourage treatment compliance, and adherence to the protocol procedures. All patients should adhere to the visit schedule as specified in Table 2 and 11. Any deviations from the planned visit schedule should be documented.

All study visits should start preferably in the morning. Patients should be instructed to not take their trial medication at scheduled clinic visit days (from visit 2 to EoT visit). Trial medication must be administered during the clinic study visit as the time points specified in Table 6.

If any visit after the first dosing visit (visit 2) is rescheduled or missed, subsequent visits should follow the original visit date. The total treatment period (visit 2 to EoT visit) should be 12 weeks.

Unscheduled visits may be arranged, and it will be at the discretion of the investigator in order to check the safety or for other reasons.

ECGs should be recorded before blood samples are taken. Post-dose ECGs should be recorded before the PK sample is taken.

Eye exams should be completed at the study site or at another healthcare facility.

For patients participating in the trial remotely via the DCT model, study visits and procedures (except eye exams) will be performed by a MRN deployed by a CRO authorized by BI. Home visits may also be performed by an appropriately qualified member of the investigational site staff (e.g. investigator, study nurse). Investigative sites may also contract their own mobile research nurse to perform the study visits at the patient's home. Phone visits should be conducted by site staff.

The remote visits must adhere to the visit schedule as specified in Table 2. The EoS visit by telephone will be completed by the study site staff.

In the event of force majeure or other disruptive circumstances (e.g. pandemic, war) the investigational plan as per this clinical trial protocol may not be feasible at a site. With the consent of the patient, sponsor and investigator may agree on alternative, back-up or rescue methodology which may include but will not be limited to virtual patient visits and assessments, and home healthcare nurse visits. The implementation of these measures will depend on patient's consent, operational feasibility, local law and regulations. If alternative methodology is implemented, the deviations from the original plan will be precisely documented.

### First Morning Void—Urine Collection:

First morning void sample is collected during the screening period to obtain the UPCR needed to determine eligibility for the trial. The first morning void is the urination after the patient wakes up to start their day. If the patient goes back to sleep after urinating early in the morning e.g. at 4 am, this void does not need to be collected and this does not need to be documented. This applies also to any urinations earlier during the night in patients who have nocturia. However, if the patient is an early riser and gets up ‘for good’ e.g. at 4 am, this would qualify as their first morning void. There may be cases when a patient might go back to bed after their usual rising time. In those cases, the void after the usual rising time constitutes the first morning void. The FMV sample can be brought to the site by the patient or may be shipped to the study site via a courier when possible.

Patients can sign a separate screening consent for the purpose of collecting the first morning void urine sample during the screening period or provide a verbal consent. Site will process the urine samples and send them to central lab for UPCR analysis. The UPCR value obtained from the first morning void urine sample will be used to assess eligibility of the patient for the trial. Site staff should ensure sufficient time is available to get the UPCR results from central lab to complete the screening period within the protocol allowed window. If the patient did not meet the inclusion criteria for UPCR from the first morning void, the patient may repeat the assessment once, and the UPCR value obtained from the 2^{nd} first morning void urine sample can be used to determine eligibility for the study.

### 24-Hour Urine Collection:

The schedule for 24-hour urine collection and the time points for dispensing urine collection containers are provided in Table 2. The start and end time of the 24-hour urine collection will be recorded. Patients will be asked to empty their bladders outside the container before start of sampling and into the container during and at the end of the 24-hour collection period. Patients will receive detailed instructions on the collection, storage, and transportation of the urine samples. Processing and analyses of the 24-hour urine samples will be described in the laboratory manual.

The 24-hour urine samples where collection started the day before the study visits should be brought to the clinic by the patient for the respective study visits. Other 24-hour urine samples (collected during the screening period) can be brought to the site by the patient or may be shipped to the study site via a courier when possible.

During the screening period, patient will collect 24-hour urine samples at two separate occasions. The 24-hour urine samples should be collected only after confirmation that the patient met all the eligibility criteria for the trial, and the collections should be close to visit 2 as much as possible. Visit 2 must be rescheduled if UPCR data is not available from at least one 24-hour urine sample.

For visits 3, EoT, and FUP1, one 24-hour urine sample will be collected. Collection should start preferably the day before the respective study visit and end on the day of the visit.

Visit 2 must be rescheduled if at least one of the 24-hour urine sample is not collected. The EoT visit must be rescheduled if the 24-hour urine sample is not collected before the visit. Visits must be rescheduled as soon as possible.

For patients participating in the trial remotely via the DCT model, urine collection containers for all the study visits will be sent to the patient's home, and the urine samples will be shipped to the study site via a courier. If patients live close to the study site, they may also pick up the urine collection containers from the study site and bring the 24-hour urine samples to the study site.

If possible, patients should be reminded (e.g. via telephone) during the screening period and ahead of the applicable visits to collect the 24-hour urine samples.

Collection of 24-hour urine samples may be repeated for logistical reasons (e.g. samples lost or patient could not complete the 24-hour collection) or technical issues (e.g. sample not fit for analysis).

### Pregnancy Tests

All WOCBP will undergo serum pregnancy test at the screening visit (visit 1). Patients who test positive for the serum pregnancy test will be excluded from the trial. If the patient is a WOCBP, the status of the menstrual cycle should be assessed before the administration of the first dose of trial medication (visit 2): in case of a delayed or missed period, the PI should use their clinical judgement and the results of the pregnancy test at visit 2 to assess the participants pregnancy status and confirm eligibility before intake of trial drug.

Urine pregnancy tests will be done at all study visits starting from visit 2 (except visit 3 and phone visits). If the urine pregnancy test is positive, trial medication will be stopped and a serum pregnancy test will be performed to confirm pregnancy. If the serum pregnancy test is positive, the patient will be discontinued from the trial. EoT visit will be scheduled as soon as possible, and patient should complete the EoS visit. If serum pregnancy is negative, patient may continue in the trial and resume treatment with trial medication. If the urine pregnancy test is positive at the first dosing visit (visit 2), the patient must not be dosed unless the serum pregnancy test is negative.

### 4.2 Details of Trial Procedures at Selected Visits

### 4.2.1 Screening and Run-In Period

Trial procedures to be performed during the screening period can be found in Table 2. Screening period is defined as the time between the date of informed consent (or the screening consent) to the date of first dose (visit 2). No trial procedures should be performed until the patient has consented to take part in the trial. The separate screening consent is to allow collection of the first morning void urine sample to obtain the UPCR needed to check eligibility criteria. Each patient will be assigned a unique patient number and enrollment will be recorded in eCRF.

For patients participating in the trial via the DCT model, the consenting process may be completed electronically (via eConsent) within the DCT Platform. Screening consent when applicable will be administered outside the DCT Platform. Prior to initiating the eConsent process, eligibility of potential patients will be assessed after review of medical records by the investigator or site staff. Once this preliminary eligibility is confirmed, a study informed consent discussion will be scheduled. The informed consent materials will be presented in the DCT Platform. The patient will review the documents and during this telephone call with the investigator or designee will have the opportunity to discuss the study, and have their questions answered. If patient agrees to participate in the trial, an electronic signature will be obtained.

Once the patient has consented to collect the first morning void urine sample for screening purposes, the patient is considered to be enrolled in the trial. The patient should be recorded on the enrollment log and be registered in the IRT.

Baseline conditions, medical history, and eligibility criteria will be assessed at visit 1. Concomitant therapy and AE (if any) will be recorded. At the conclusion of visit 1, patients should receive instructions on procedures to be followed during the screening period.

If all the eligibility criteria are met, randomization will be completed by calling the IRT. Randomization will initiate trial medication shipment to the patient and visit 2 will be scheduled. For more information on medication administration.

Screening period may be extended for administrative reasons with approval from the CT Manager.

If screening period exceeds 30 days, the investigator should review the laboratory reports from the screening visit (visit 1) and make a determination if any labs specified in the protocol must be repeated before the first dosing visit. If deemed necessary, test samples should be drawn and sent to the central laboratory.

### Discontinuation During the Screening Period

If patients discontinue from the trial during the screening period, no additional study visits are required, and they will be marked as screen failures. Patient will be registered as a screen failure in IRT.

### Rescreening and Retesting

Patients who screen failed due to a reason that was reversible and has since been resolved or those who were screen failed for administrative reasons (e.g., extended travel, life events) may be rescreened once with approval from the CT Manager or designee.

If the investigator believes that a lab test result is due to an error or other extenuating circumstances, the lab test can be repeated once without the patient having to be rescreened.

### 4.2.2 Treatment Period.

Treatment period will begin at visit 2 and will continue for 12 weeks. Procedures to be completed at each study visit can be found in the Table 2. There will be three phone call visits during the treatment period: visit.

Unscheduled visits may be arranged if necessary. Procedures completed during an unscheduled visit will depend on the circumstances under which the visit was scheduled, and at the discretion of the investigator.

EoT must be rescheduled if the 24-hour urine sample is not collected for UPCR measurements. After completion of the treatment period, the patient will enter the 30-day follow-up period.

### Discontinuation During the Treatment Period

For discontinued patients, EoT visit must be scheduled as soon as possible. After completion of the EoT visit, patient will complete both follow-up visits (FUP1 and EoS).

### 4.2.3 Follow-Up Period and Trial Completion

The 30-day follow-up period extends from the EoT visit until the EoS phone call visit. The FUP1 visit should be scheduled 7 days after the EoT visit, and EoS phone call visit should be scheduled 30 days after the EoT visit. The EoS visit will be a telephone visit for all patients. For patients participating in the trial remotely, the study site staff will complete EoS visit by telephone. Procedures to be completed at the follow-up visits can be found in Table 2. Investigator should ensure that eye exams (if applicable) are completed as part of the EoS visit and the results are reviewed.

The last study visit will be the EoS visit and this will mark the end of observation period, and the patient has completed the trial.

### 5. Statistical Methods and Determination of Sample Size

### 5.1 Null and Alternative Hypotheses

No confirmatory testing will be performed and hence no null and alternative hypotheses are defined since this is a non-confirmatory study.

### 5.2 Planned Analyses

### 5.2.1 General Considerations

The following analysis sets will be defined for statistical analyses:
Entered Set (ES): This patient set includes all patients who signed informed consent. The ES will be used for the analysis of patient disposition.
Randomized Set (RS): This patient set includes all patients who signed the informed consent form and were also randomized, regardless of whether the patient was treated with trial medication or not.
Treated Set (TS): This patient set includes all patients who received at least one dose of trial medication. The TS is used for safety analyses as well as demographics and baseline characteristics.
Full Analysis Set (FAS): This patient set includes all patients who were randomized and treated with evaluable measurements UPCR at baseline and at least one UPCR measurement after first dose. The FAS is the main analysis set for the analysis of efficacy.
Pharmacokinetic Analysis Set (PKS): This patient set includes all patients in the TS who provide at least one PK endpoint that was not excluded due to protocol violation relevant to the evaluation of PK or due to PK non-evaluability.

For the analysis of efficacy, patients will be analyzed as randomized, without regard to any treatment changes.

### 5.2.2 Handling of Intercurrent Events

An intercurrent event (ICE) is defined as an event of,
early discontinuation,
lost to follow-up, or
death.

The strategies for handling intercurrent events in this trial are as follows:
Hypothetical estimand: assuming all subjects remained adherent to the assigned trial medication and the study protocol. This strategy will include all data collected until time of an ICE.
Treatment policy estimand: using all available data including data collected after an ICE.

### 5.2.3 Primary Endpoint Analyses

An exploratory inferential analysis for the primary endpoint, in terms of proportion of patients achieving at least 25% UPCR reduction relative to baseline at 12 weeks, will be conducted by providing 95% confidence intervals from each treatment group. The primary estimand of interest is the treatment effect assuming all subjects remained adherent to the assigned trial medication and the study protocol using a hypothetical approach, i.e., study drug is taken as directed. This analysis will include all data collected until time of an ICE.

In addition, an analysis of variance (ANOVA) model (for the UPCR change from baseline at week 12) will be used to see difference across arms. Dose-response relationship based on reduction in log of UPCR may be explored using graphical approach.

### Sensitivity Analyses

Sensitivity analyses to be conducted to assess the robustness of the primary analysis outcome will be described in the trial statistical analysis plan (TSAP).

### Supplemental Analysis of Primary Endpoint

An additional assessment of the primary endpoint will be conducted using the treatment policy estimand, i.e., effectiveness/intention to treat. The treatment policy estimand will use all available data including data collected after an ICE. All attempts will be made to collect all data per protocol.

### Subgroup Analyses

No subgroup analysis planned.

### 5.2.4 Secondary Endpoint Analyses

All secondary endpoints in except pharmacokinetic parameters of the TRPC6 inhibitor will be analyzed by using descriptive statistics and figures.

Further details will be given in the TSAP.

### 5.2.5 Further Endpoint Analyses

The following further endpoints will be described by using descriptive statistics.
Change in eGFR from visit 2 to week 12 and 13
Change in UACR relative to baseline at weeks 4, 8, 12 and 13
Change in UPCR relative to baseline at weeks 4, 8, 12, and 13.

If additional statistical analyses are needed, methodology similar to those described for the primary and secondary analyses will be utilized. Final details will be covered in the TSAP.

### 5.2.6 Safety Analyses

Adverse events will be coded using the Medical Dictionary for Drug Regulatory Activities (MedDRA). Standard BI summary tables and listings will be produced. All adverse events with an onset between start of treatment and end of study, which include a 5-day period after last dose of trial medication, will be assigned to the on-treatment period for evaluation.

All treated patients (i.e., all patients who received at least one dose of trial medication) will be included in the safety analysis. In general, safety analyses will be descriptive in nature and will be based on BI standards. No hypothesis testing is planned.

Statistical analysis and reporting of adverse events will concentrate on treatment-emergent adverse events, i.e. all adverse events occurring between start of treatment and end of the study. Adverse events that start before first intake of trial medication and deteriorate under treatment will also be considered as ‘treatment-emergent’.

Frequency, severity, and causal relationship of adverse events will be tabulated by system organ class and preferred term after coding according to the current version of the MedDRA at the database lock.

Laboratory data will be analyzed both quantitatively as well as qualitatively. The latter will be done via comparison of laboratory data to their reference ranges. Values outside the reference range as well as values defined as clinically relevant will be summarized. Treatment groups will be compared descriptively with regard to distribution parameters as well as with regard to frequency and percentage of patients with abnormal values or clinically relevant abnormal values.

Vital signs, physical examinations, or other safety-relevant data observed at screening, baseline, during the course of the study and at the end-of-study evaluation will be assessed with regards to possible changes compared to findings before start of treatment.

### 5.2.7 Other Analyses

Unblinded exploratory data analysis on pharmacokinetic, pharmacodynamic, biomarker and/or other trial data will be performed during the conduct of the trial.

### 5.2.8 Interim Analyses

No formal interim analysis is planned.

### 5.3 Handling of Missing Data

No missing data will be imputed in the UPCR, UACR, and urinary protein excretion, analyses. Handling of missing PK data will be performed according to the relevant Corporate Procedure. PK parameters that cannot be reasonably calculated based on the available drug concentration-time data will not be imputed. Further details will be specified in the TSAP.

### 5.4 Randomization

The study will be performed as a double-blind design with respect to the 3 different doses of the TRPC6 inhibitor and placebo in a 1:1:1:1 ratio stratified by use of corticosteroids. Patients will be randomized in blocks to double-blind treatment via the IRT system.

The sponsor will arrange for the randomization as well as packaging and labelling of trial medication. The randomization list will be generated using a validated system that uses a pseudo-random number generator and a supplied seed number so that the resulting allocation is both reproducible and non-predictable.

### 5.5 Determination of Sample Size

To explore the clinical principle of the TRPC6 inhibitor it is planned to include a total of 60 patients with FSGS. The planned sample size is not based on a power calculation. The size of 15 patients per treatment arm is considered to be sufficient to detect differences between the different treatment groups and placebo in terms of the primary endpoint.

The goal of the trial is to determine if the difference in UPCR response between at least one dose after baseline and placebo is greater than 25%. Assuming that approximately 40%, 30%, 20% of patients in 80 mg, 40 mg, 20 mg treatment groups, respectively, achieve at least 25% UPCR reduction at 12 weeks while 9% of patients in placebo group achieve 25% UPCR reduction, the proposed sample size provides 73.4% probability for the difference in responder rates between at least one treatment arm and placebo to be greater than 25%.

What is claimed is:

1. A method for reducing the level of proteinuria and/or preserving renal function in a patient having focal segmental glomerulosclerosis (FSGS), comprising administering to the patient a pharmaceutically effective amount of a compound of formula (I), 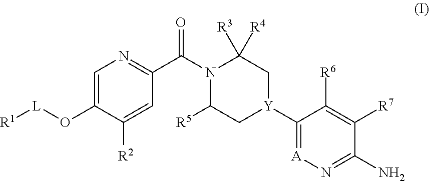
wherein
L is absent or is methylene or ethylene;
Y is CH or N;
A is CH or N;
R¹ is selected from the group consisting of:
C₁₋₆alkyl optionally substituted with 1 to 3 groups independently selected from the group consisting of halo, C₃₋₆cycloalkyl and OC₃₋₆cycloalkyl;
phenyl optionally substituted with 1 to 3 groups independently selected from the group consisting of CF₃, halo, C₃₋₆cycloalkyl, OC₃₋₆cycloalkyl, and OC₁₋₆alkyl; wherein said OC₁₋₆alkyl may be optionally substituted with one to three halo; and
C₃₋₆cycloalkyl optionally substituted with 1 to 3 groups independently selected from the group consisting of halo and C₁₋₆alkyl optionally substituted with 1 to 3 halo;
R² is selected from the group consisting of H, C₁₋₆alkyl, OCF₃, C₃₋₆cycloalkyl, OC₁₋₆alkyl, and OC₃₋₆cycloalkyl;
R³ is selected from the group consisting of H, C₁₋₆alkyl, C₃₋₆cycloalkyl, and OC₃₋₆cycloalkyl; wherein each of the C₁₋₆alkyl, C₃₋₆cycloalkyl, or OC₃₋₆cycloalkyl of the R³ group may independently be optionally substituted with one to three groups each independently selected from the group consisting of halo, OH, OC₁₋₆alkyl, SC₁₋₆alkyl, and N(C₁₋₆alky)₂; and wherein one to three carbon atoms of the C₁₋₆alkyl of the R³ group may optionally be replaced one or two moieties selected from the group consisting of NH, N(C₁₋₆alkyl), O, and S;
R⁴ and R⁵ are each independently selected from the group consisting of H and C₁₋₆alkyl; or
R³ and R⁴ together with the atom to which they are attached may join to form a 3-membered carbocyclyl ring; or
R³ and R⁵ together with the atoms to which they are attached may join to form a 3- to 9-membered bicyclic ring, wherein said 3- to 9-membered bicyclic ring may optionally contain one to three heteroatoms selected from the group consisting of N, O, and S;
R⁶ is selected from the group consisting of H, C₁₋₆alkyl, CN, CF₃, OCF₃, C₃₋₆cycloalkyl, OC₁₋₆alkyl, and OC₃₋₆cycloalkyl;
R⁷ is selected from the group consisting of H and OC₁₋₆alkyl;
or a pharmaceutically acceptable salt thereof.

2. The method according to claim 1, wherein
R¹ is selected from the group consisting of:
C₁₋₆alkyl optionally substituted with 1 to 3 groups independently selected from the group consisting of halo and C₃₋₆cycloalkyl;
phenyl optionally substituted with 1 to 3 groups independently selected from the group consisting of CF₃, halo, OC₃₋₆cycloalkyl, and OC₁₋₆alkyl; wherein said OC₁₋₆alkyl may be optionally substituted with one to three halo; and
C₃₋₆cycloalkyl optionally substituted with 1 to 3 halo groups;
R² is OC₁₋₆alkyl;
R³ is selected from the group consisting of H and C₁₋₆alkyl optionally substituted with OH or OC₁₋₆alkyl,
R⁴ is H;
R⁵ is H; or
R³ and R⁴ together with the atom to which they are attached may join to form a 3-membered carbocyclyl ring; or
R³ and R⁵ together with the atoms to which they are attached may join to form a 3- to 9-membered bicyclic ring, wherein said 3- to 9-membered bicyclic ring may optionally contain one to three heteroatoms selected from the group consisting of N and 0;
R⁶ is selected from the group consisting of H, C₁₋₆alkyl, OC₁₋₆alkyl, and OC₃₋₆cycloalkyl; and
R⁷ is selected from the group consisting of H and OC₁₋₆alkyl;
or a pharmaceutically acceptable salt thereof.

3. The method according to claim 1, wherein
A is CH and Y is N; or
A is CH and Y is CH; or
A is N and Y is CH;
or a pharmaceutically acceptable salt thereof.

4. The method according to claim 1, wherein
R¹ is phenyl optionally substituted with a group selected from the group consisting of CF₃, halo, OC₃₋₆cycloalkyl, and OC₁₋₆alkyl; wherein the OC₁₋₆alkyl may be optionally substituted with one to three halo;
R² is OC₁₋₆alkyl;
R³ is selected from the group consisting of H and C₁₋₆alkyl optionally substituted with OH or OC₁₋₆alkyl;
R⁴ is H;
R⁵ is H; or
R³ and R⁴ can together with the atom to which they are attached may join to form a 3-membered carbocyclyl ring, or
R³ and R⁵ together with the atoms to which they are attached may join to form a 3- to 9-membered bicyclic ring, wherein said 3- to 9-membered bicyclic ring may optionally contain one to three heteroatoms selected from the group consisting of N and 0;
R⁶ is selected from the group consisting of H, C₁₋₆alkyl, OC₁₋₆alkyl, and OC₃₋₆cycloalkyl;
R⁷ is selected from the group consisting of H and OC₁₋₆alkyl;
or a pharmaceutically acceptable salt thereof.

5. The method according to claim 1, wherein
R¹ is phenyl optionally substituted with a group selected from the group consisting of CF₃, OCF₃, F, and methoxy;
R² is selected from the group consisting of methoxy or ethoxy;
R³ is selected from the group consisting of H, C₁₋₆alkyl, 2-hydroxymethyl, methoxymethyl, and 1-hydroxyethyl;
R⁴ is H;
R⁵ is H; or
R³ and R⁵ together with the atoms to which they are attached may join to form a 3- to 9-membered bicyclic ring, wherein said 3- to 9-membered bicyclic ring may optionally contain one to three heteroatoms selected from the group consisting of N, O, and S;
R⁶ is selected from the group consisting of H, methyl, methoxy, ethoxy, propoxy, and cyclopropyloxy; and
R⁷ is selected from the group consisting of H and methoxy;
or a pharmaceutically acceptable salt thereof.

6. The method according to claim 1, wherein the compound of formula (I) is selected from the group consisting of:
[4-(6-Amino-pyridazin-3-yl)-piperidin-1-yl]-[5-(4-isopropoxy-phenoxy)-4-methoxy-pyridin-2-yl]-methanone,
[4-(6-Amino-pyridazin-3-yl)-piperidin-1-yl]-(4-methoxy-5-phenoxy-pyridin-2-yl)-methanone,
[4-(6-Amino-pyridazin-3-yl)-piperidin-1-yl]-[5-(4-fluoro-phenoxy)-4-methoxy-pyridin-2-yl]-methanone,
[4-(6-Amino-pyridazin-3-yl)-piperidin-1-yl]-[4-methoxy-5-(4-trifluoromethyl-phenoxy)-pyridin-2-yl]-methanone,
[4-(6-Amino-pyridazin-3-yl)-piperidin-1-yl]-[5-(4-chloro-phenoxy)-4-methoxy-pyridin-2-yl]-methanone,
[4-(6-Amino-pyridazin-3-yl)-piperidin-1-yl]-[5-(4-cyclopropoxy-phenoxy)-4-methoxy-pyridin-2-yl]-methanone,
[4-(6-Amino-pyridazin-3-yl)-piperidin-1-yl]-[5-(4-fluoro-benzyloxy)-4-methoxy-pyridin-2-yl]-methanone,
[4-(6-Amino-pyridazin-3-yl)-piperidin-1-yl]-[4-methoxy-5-(4-methoxy-phenoxy)-pyridin-2-yl]-methanone,
[4-(6-Amino-pyridazin-3-yl)-piperidin-1-yl]-[5-(4-difluoromethoxy-phenoxy)-4-methoxy-pyridin-2-yl]-methanone,
[4-(6-Amino-pyridazin-3-yl)-piperidin-1-yl]-[5-(2-fluoro-benzyloxy)-4-methoxy-pyridin-2-yl]-methanone,
[4-(6-Amino-pyridazin-3-yl)-piperidin-1-yl]-[4-methoxy-5-(4-trifluoromethoxy-phenoxy)-pyridin-2-yl]-methanone,
[4-(6-Amino-pyridazin-3-yl)-piperidin-1-yl]-[5-(phenoxy)-4-ethoxy-pyridin-2-yl]-methanone; and
[4-(6-Amino-pyridazin-3-yl)-piperidin-1-yl]-[5-(4-fluoro-phenoxy)-4-ethoxy-pyridin-2-yl]-methanone,
or a pharmaceutically acceptable salt thereof.

7. The method according to claim 1, wherein the compound of formula (I) is selected from the group consisting of:
[4-(6-Amino-4-methyl-pyridazin-3-yl)-piperidin-1-yl]-[5-(4-fluoro-phenoxy)-4-methoxy-pyridin-2-yl]-methanone,
[4-(6-Amino-4-methyl-pyridazin-3-yl)-piperidin-1-yl]-(4-methoxy-5-phenoxy-pyridin-2-yl)-methanone,
[4-(6-Amino-4-methoxy-pyridazin-3-yl)-piperidin-1-yl]-[5-(4-fluoro-phenoxy)-4-methoxy-pyridin-2-yl]-methanone,
[4-(6-Amino-4-methoxy-pyridazin-3-yl)-piperidin-1-yl]-[4-methoxy-5-(4-trifluoromethyl-phenoxy)-pyridin-2-yl]-methanone,
[4-(6-Amino-4-methoxy-pyridazin-3-yl)-piperidin-1-yl]-[4-methoxy-5-(4-methoxy-phenoxy)-pyridin-2-yl]-methanone,
[4-(6-Amino-4-ethoxy-pyridazin-3-yl)-piperidin-1-yl]-[4-methoxy-5-(phenoxy)-pyridin-2-yl]-methanone,
5-Ethoxy-6-(1-{4-methoxy-5-[4-(trifluoromethyl)phenoxy]pyridine-2-carbonyl}piperidin-4-yl)pyridazin-3-amine, and
6-(1-{4-Methoxy-5-[4-(trifluoromethyl)phenoxy]pyridine-2-carbonyl}piperidin-4-yl)-5-methylpyridazin-3-amine,
or a pharmaceutically acceptable salt thereof.

8. The method according to claim 1, wherein the level of proteinuria in the patient is reduced.

9. The method according to claim 8, wherein the level of proteinuria in the patient is reduced at least 25% based on a 24-hour urine protein-creatinine ratio (UPCR) relative to baseline at week 12.

10. The method according to claim 9, wherein the renal function is preserved in the patient.

11. The method according to claim 10, wherein the estimated glomerular filtration rate (eGFR) is preserved in the patient.

12. The method according to claim 1, wherein the TRPC inhibitor is administered to the patient in an amount of 10 mg, or 12.5 mg, or 15 mg, or 17.5 mg, or 20 mg, or 22.5 mg, or 25 mg, or 27.5 mg, or 30 mg, or 32.5 mg, or 35 mg, or 37.5 mg, or 40 mg, or 42.5 mg, or 45 mg, or 47.5 mg, or 50 mg, or 52.5 mg, or 55 mg, or 57.5 mg, or 60 mg, or 62.5 mg, or 65 mg, or 67.5 mg, or 70 mg, or 72.5 mg, or 75 mg, or 77.5 mg, or 80 mg, or 82.5 mg, or 85 mg, or 87.5 mg, or 90 mg.
